(19) United States
(12) Patent Application Publication
(10) Pub. No.: US 2021/0347873 A1
(43) Pub. Date: Nov. 11, 2021

(54) BLYS ANTIBODY, PREPARATION METHOD THEREFOR AND APPLICATION THEREOF

(71) Applicant: SHANGHAI PHARMAEXPLORER CO., LTD., Shanghai Pilot Free Trade Zone (CN)

(72) Inventors: Xiaodan CAO, Shanghai (CN); Yingying HU, Shanghai (CN); Fang REN, Shanghai (CN); Shiyong GONG, Shanghai (CN); Jing GONG, Shanghai (CN); Qiang LV, Shanghai (CN); Hongzhuan GU, Shanghai (CN); Beilei SHI, Shanghai (CN); Xiaohui SHAO, Shanghai (CN); Xiaofen LV, Shanghai (CN); Stewart LEUNG, Shanghai (CN); Lile LIU, Shanghai (CN)

(21) Appl. No.: 16/315,519

(22) PCT Filed: Jul. 5, 2017
(86) PCT No.: PCT/CN2017/091839

§ 371 Date: Jan. 4, 2019

(30) Foreign Application Priority Data
Jul. 6, 2016 (CN) 201610527996.0

(51) Int. Cl. C07K 16/24

(52) U.S. Cl. CPC C07K 16/241; C07K 2317/565; C07K 2317/34; C07K 2317/567; C07K 2317/73; C07K 2317/92; C07K 2317/76

(57) ABSTRACT

A BLyS antibody, preparation method therefor and application thereof, the BLyS antibody comprising one or more of CDR1, CDR2 and CDR3 of a heavy chain variable region of the BLyS antibody, and/or one or more of CDR1, CDR2 and CDR3 of a light chain variable region of the BLyS antibody. An amino acid sequence of the antibody is shown in a sequence listing. The BLyS antibody has a high affinity, and may observably and effectively seal a BLyS protein at a protein level and a cell level, and prevent the BLyS protein from binding to a receptor. The BLyS antibody lacks a cross-reaction with homologous protein antigens such as human APRIL, and enjoys good biological activity. The BLyS antibody may inhibit the proliferation of human BLyS-induced mouse B cells and may therefore be used for preparing a drug for preventing or treating diseases associated with BLyS expression or dysfunction.

This application claims priority of Chinese Patent Application No. 201610527996.0 filed on Jul. 6, 2016. The entire content of the aforementioned application is hereby incorporated by reference.

### FIELD OF INVENTION

The present invention relates to the antibody, and more specifically, relates to BlyS antibody, preparation method therefor and application thereof.

### PRIOR ARTS

Autoimmune disease is a disease that the body's own normal organs, tissues and cells are attacked by immune system. As a chronic disease, it is incurable but controllable, and the body will become very sick once people suffering from the disease. This leads to a sharp reduction in the quality of life and the high medical cost will result in a heavy burden on patients and their families, and even the society. Common autoimmune diseases include systemic lupus erythematosus (SLE), rheumatoid arthritis (RA) and the like. SLE may affect various organs, and it is impossible to predict when the disease will onset so far and the natural course of the disease is characterized by the alternation of aggravation and remissions. Global average prevalence rate of SLE is 12-39 patients per hundred thousand people, and the prevalence rate of SLE in China is 30-70 patients per one hundred thousand people, having the second highest rate in the world and next to black people's 100 patients per one hundred thousand people. SLE usually occurs in young women, and 90% of patients are female. RA is a long-term, continuous disease that mainly affects the joints. It usually causes joint fever, swelling and pain, which can lead to the surface erosion and destruction of the joints, and even cause limb deformity in severe cases. According to statistics, the incidence of RA is about 0.3%. In recent years, the incidence of autoimmune disease has gradually increased in China and even in the world. Due to the vast territory and large population in China, the calculated number of patients based on this incidence is quite large. However, there is still a lack of effective intervention and treatment with small side effects and strong specificity for blocking the damage of target organ in early phase and thus improve the prognosis of patients.

Studies have shown that increased expression of B lymphocyte stimulator (BLyS) can be detected in patients with autoimmune diseases and B-cell neoplasms. For instance, the expression of BLyS in serum is elevated in various groups of patients with SLE, and it is associated with the production of autoimmune antibody and disease activity index [Stohl et al. 2003, Arthritis Rheum, 48 (12): 3475; Petri et al. 2008, Arthritis Rheum, 58 (8): 2453]. High expression level of BLyS is also found in synovial fluid of patients with RA [Tan et al. 2003, Arthritis Rheum, 48 (4): 982]. The correlation between BLyS overexpression and the clinical manifestations of these autoimmune diseases suggests that regulation of the expression level of BLyS may be a new approach for the treatment of these diseases.

BLyS, also known as BAFF, THANK, Tall-1, TNFSF13B or zTNF4, is a member of the tumor necrosis factor (TNF) ligand superfamily [Baker et al. 2003, Arthritis Rheum, 48(11):3253]. BLyS is expressed as a membrane-bound isoform with 285 amino acids of type II transmembrane protein or a soluble isoform with 152 amino acids after cleavage. BLyS is expressed on monocytes, macrophages and dendritic cells and is up-regulated by the stimulation of interferon-γ and interleukin-10. Recombinant human BLyS enhances B cell proliferation and antibody secretion by binding to the major receptors on the surface of B cells in vitro. Recombinant human BLyS leads to splenic proliferation in mice primarily due to an increase in the number of mature B cells in vivo. Injection of BLyS into mice also results in an increase of antibody concentration in the serum as well as an increase in T-cell dependent and antigen independent humoral immunity. Overexpression of BLyS can induce abnormally high level of antibodies expression which leading to SLE, RA and other autoimmune diseases.

In recent years, monoclonal antibody drugs have been widely used in the treatment of tumors, autoimmune diseases and the like due to their advantages of strong specificity, high curative effect and small side effects. The annual global sales of monoclonal antibody drugs have been increased from $300 million in 1997 to $66.3 billion in 2012, becoming one of the fastest-growing and most profitable fields in biopharmaceutical industry which has broad space of development. Currently, Belimumab, a monoclonal antibody against BLyS, is the first monoclonal antibody approved by FDA in the past half century, which is of great significance.

Although there are still some problems with Belimumab, as a monoclonal antibody for SLE treatment, the monoclonal antibodies have stronger targeting ability and significantly fewer side effects than immunosuppressants such as cyclophosphamide and hormones. It is estimated that the market has great potential and the sales of SLE drugs will reach $3.9 billion by 2022. Therefore, it is urgent to obtain new, safer and more effective anti-BLyS antibodies for the treatment of autoimmune diseases such as SLE.

Content of the Present Invention

Technical problem to be solved herein is to provide a high-affinity and high-specificity humanized or fully human BLyS antibody, a preparation method and a use thereof for overcoming the lack of effective and safe BLyS antibodies at current stage. The BLyS antibodies of the present invention have high affinity for BLyS, which can inhibit the binding of BLyS to its receptor and inhibit the proliferation of BLyS-induced mouse B lymphocytes. The BLyS antibodies herein lacks a cross-reaction with homologous protein antigens of BLyS such as human APRIL and thus they can be used in manufacturing a medicament for preventing or treating autoimmune diseases or tumors, which are associated with abnormal expression or dysfunction of BLyS.

The inventors obtained the lead antibody of BLyS by employing phage display and hybridoma technique. After preliminary production, purification and identification of the lead antibody, the BLyS antibody which comprises outstanding bioactive properties such as high affinity (affinity KD<5×10⁻⁹M), effectively blocking the binding of BLyS to its receptor, and lacking a cross-reaction with BLyS homologous protein such as human APRIL, can be obtained. Subsequently the amino acid sequences of the heavy chain variable region and the light chain variable region of BLyS antibody can be obtained by using molecular biological methods.

The present invention provides an isolated protein, which comprises complementary determining regions (CDR or CDRs) of BLyS antibody: one or more of the heavy chain CDR1, heavy chain CDR2 and heavy chain CDR3, and/or one or more of the light chain CDR1, light chain CDR2, and light chain CDR3, wherein the heavy chain CDR1 contains amino acid sequences represented by SEQ ID No.2, SEQ ID No.10, SEQ ID No.18, SEQ ID No.26, SEQ ID No.34, SEQ ID No.42, SEQ ID No.50, SEQ ID No.58, SEQ ID No.66, SEQ ID No.74, SEQ ID No.82, SEQ ID No.90 or SEQ ID No.98; the heavy chain CDR2 contains amino acid sequences represented by SEQ ID No.3, SEQ ID No.11, SEQ ID No.19, SEQ ID No.27, SEQ ID No.35, SEQ ID No.43, SEQ ID No.51, SEQ ID No.59, SEQ ID No.67, SEQ ID No.75, SEQ ID No.83, SEQ ID No.91 or SEQ ID No.99; the heavy chain CDR3 contains amino acid sequences represented by SEQ ID No.4, SEQ ID No.12, SEQ ID No.20, SEQ ID No.28, SEQ ID No.36, SEQ ID No.44, SEQ ID No.52, SEQ ID No.60, SEQ ID No.68, SEQ ID No.76, SEQ ID No.84, SEQ ID No.92 or SEQ ID No.100; the light chain CDR1 contains amino acid sequences represented by SEQ ID No.6, SEQ ID No.14, SEQ ID No.22, SEQ ID No.30, SEQ ID No.38, SEQ ID No.46, SEQ ID No.54, SEQ ID No.62, SEQ ID No.70, SEQ ID No.78, SEQ ID No.86, SEQ ID No.94 or SEQ ID No.102; the light chain CDR2 contains amino acid sequences represented by SEQ ID No.7, SEQ ID No.15, SEQ ID No.23, SEQ ID No.31, SEQ ID No.39, SEQ ID No.47, SEQ ID No.55, SEQ ID No.63, SEQ ID No.71, SEQ ID No.79, SEQ ID No.87, SEQ ID No.95 or SEQ ID No.103; the light chain CDR3 contains amino acid sequences represented by SEQ ID No.8, SEQ ID No.16, SEQ ID No.24, SEQ ID No.32, SEQ ID No.40, SEQ ID No.48, SEQ ID No.56, SEQ ID No.64, SEQ ID No.72, SEQ ID No.80, SEQ ID No.88, SEQ ID No.96 or SEQ ID No.104;

Or, the amino acid sequences of the heavy chain CDR1 is at least 80% identical to the amino acid sequences represented by SEQ ID No.2, SEQ ID No.10, SEQ ID No.18, SEQ ID No.26, SEQ ID No.34, SEQ ID No.42, SEQ ID No.50, SEQ ID No.58, SEQ ID No.66, SEQ ID No.74, SEQ ID No.82, SEQ ID No.90 or SEQ ID No.98; the amino acid sequences of the heavy chain CDR2 is at least 80% identical to the amino acid sequences represented by SEQ ID No.3, SEQ ID No.11, SEQ ID No.19, SEQ ID No.27, SEQ ID No.35, SEQ ID No.43, SEQ ID No.51, SEQ ID No.59, SEQ ID No.67, SEQ ID No.75, SEQ ID No.83, SEQ ID No.91 or SEQ ID No.99; the amino acid sequences of the heavy chain CDR3 is at least 80% identical to the amino acid sequences represented by SEQ ID No.4, SEQ ID No.12, SEQ ID No.20, SEQ ID No.28, SEQ ID No.36, SEQ ID No.44, SEQ ID No.52, SEQ ID No.60, SEQ ID No.68, SEQ ID No.76, SEQ ID No.84, SEQ ID No.92 or SEQ ID No.100; the amino acid sequences of the light chain CDR1 is at least 80% identical to the amino acid sequences represented by SEQ ID No.6, SEQ ID No.14, SEQ ID No.22, SEQ ID No.30, SEQ ID No.38, SEQ ID No.46, SEQ ID No.54, SEQ ID No.62, SEQ ID No.70, SEQ ID No.78, SEQ ID No.86, SEQ ID No.94 or SEQ ID No.102; the amino acid sequences of the light chain CDR2 is at least 80% identical to the amino acid sequences represented by SEQ ID No.7, SEQ ID No.15, SEQ ID No.23, SEQ ID No.31, SEQ ID No.39, SEQ ID No.47, SEQ ID No.55, SEQ ID No.63, SEQ ID No.71, SEQ ID No.79, SEQ ID No.87, SEQ ID No.95 or SEQ ID No.103; the amino acid sequences of the light chain CDR3 is at least 80% identical to the amino acid sequences represented by SEQ ID No.8, SEQ ID No.16, SEQ ID No.24, SEQ ID No.32, SEQ ID No.40, SEQ ID No.48, SEQ ID No.56, SEQ ID No.64, SEQ ID No.72, SEQ ID No.80, SEQ ID No.88, SEQ ID No.96 or SEQ ID No.104.

Preferably, the heavy chain CDR1 comprises amino acid sequence of SEQ ID No.2, the heavy chain CDR2 comprises amino acid sequence of SEQ ID No.3 and the heavy chain CDR3 comprises amino acid sequence of SEQ ID No.4; the heavy chain heavy chain CDR1 comprises amino acid sequence of SEQ ID No.10, the heavy chain CDR2 comprises amino acid sequence of SEQ ID No.11 and a CDR3 comprises amino acid sequence of SEQ ID No.12; the heavy chain CDR1 comprises amino acid sequence of SEQ ID No.18, the heavy chain CDR2 comprises amino acid sequence of SEQ ID No.19 and the heavy chain CDR3 comprises amino acid sequence of SEQ ID No.20; the heavy chain CDR1 comprises amino acid sequence of SEQ ID No.26, the heavy chain CDR2 comprises amino acid sequence of SEQ ID No.27 and the heavy chain CDR3 comprises amino acid sequence of SEQ ID No.28; the heavy chain CDR1 comprises amino acid sequence of SEQ ID No.34, the heavy chain CDR2 comprises amino acid sequence of SEQ ID No.35 and a CDR3 comprises amino acid sequence of SEQ ID No.36; the heavy chain CDR1 comprises amino acid sequence of SEQ ID No.42, the heavy chain CDR2 comprises amino acid sequence of SEQ ID No.43 and the heavy chain CDR3 comprises amino acid sequence of SEQ ID No.44; the heavy chain CDR1 comprises amino acid sequence of SEQ ID No.50, the heavy chain CDR2 comprises amino acid sequence of SEQ ID No.51 and the heavy chain CDR3 comprises amino acid sequence of SEQ ID No.52; the heavy chain CDR1 comprises amino acid sequence of SEQ ID No.58, the heavy chain CDR2 comprises amino acid sequence of SEQ ID No.59 and the heavy chain CDR3 comprises amino acid sequence of SEQ ID No.60; the heavy chain CDR1 comprises amino acid sequence of SEQ ID No.66, the heavy chain CDR2 comprises amino acid sequence of SEQ ID No.67 and the heavy chain CDR3 comprises amino acid sequence of SEQ ID No.68; the heavy chain CDR1 comprises amino acid sequence of SEQ ID No.74, the heavy chain CDR2 comprises amino acid sequence of SEQ ID No.75 and the heavy chain CDR3 comprises amino acid sequence of SEQ ID No.76; the heavy chain CDR1 comprises amino acid sequence of SEQ ID No.82, the heavy chain CDR2 comprises amino acid sequence of SEQ ID No.83 and the heavy chain CDR3 comprises amino acid sequence of SEQ ID No.84; the heavy chain CDR1 comprises amino acid sequence of SEQ ID No.90, the heavy chain CDR2 comprises amino acid sequence of SEQ ID No.91 and the heavy chain CDR3 comprises amino acid sequence of SEQ ID No.92; the heavy chain CDR1 comprises amino acid sequence of SEQ ID No.98, a CDR2 comprises amino acid sequence of SEQ ID No.99 and the heavy chain CDR3 comprises amino acid sequence of SEQ ID No.100;

The light chain CDR1 comprises amino acid sequence of SEQ ID No.6, the light chain CDR2 comprises amino acid sequence of SEQ ID No.7 and the light chain CDR3 comprises amino acid sequence of SEQ ID No.8; the light chain CDR1 comprises amino acid sequence of SEQ ID No.14, the light chain CDR2 comprises amino acid sequence of SEQ ID No.15 and the light chain CDR3 comprises amino acid sequence of SEQ ID No.16; the light chain CDR1 comprises amino acid sequence of SEQ ID No.22, the light chain CDR2 comprises amino acid sequence of SEQ ID No.23 and the light chain CDR3 comprises amino acid sequence of SEQ ID No.24; the light chain CDR1 comprises amino acid sequence of SEQ ID No.30, the light chain CDR2 comprises amino acid sequence of SEQ ID No.31 and the light chain CDR3 comprises amino acid sequence of SEQ ID No.32; the light chain CDR1 comprises amino acid sequence of SEQ ID No.38, the light chain CDR2 comprises amino acid sequence of SEQ ID No.39 and the light chain CDR3 comprises amino acid sequence of SEQ ID No.40; the light chain CDR1 comprises amino acid sequence of SEQ ID No.46, the light chain CDR2 comprises amino acid sequence of SEQ ID No.47 and the light chain CDR3 comprises amino acid sequence of SEQ ID No.48; the light chain CDR1 comprises amino acid sequence of SEQ ID No.54, the light chain CDR2 comprises amino acid sequence of SEQ ID No.55 and the light chain CDR3 comprises amino acid sequence of SEQ ID No.56; the light chain CDR1 comprises amino acid sequence of SEQ ID No.62, the light chain CDR2 comprises amino acid sequence of SEQ ID No.63 and the light chain CDR3 comprises amino acid sequence of SEQ ID No.64; the light chain CDR1 comprises amino acid sequence of SEQ ID No.70, the light chain CDR2 comprises amino acid sequence of SEQ ID No.71 and the light chain CDR3 comprises amino acid sequence of SEQ ID No.72; the light chain CDR1 comprises amino acid sequence of SEQ ID No.78, the light chain CDR2 comprises amino acid sequence of SEQ ID No.79 and the light chain CDR3 comprises amino acid sequence of SEQ ID No.80; the light chain CDR1 comprises amino acid sequence of SEQ ID No.86, the light chain CDR2 comprises amino acid sequence of SEQ ID No.87 and the light chain CDR3 comprises amino acid sequence of SEQ ID No.88; the light chain CDR1 comprises amino acid sequence of SEQ ID No.94, the light chain CDR2 comprises amino acid sequence of SEQ ID No.95 and a CDR3 comprises amino acid sequence of SEQ ID No.96; or, the light chain light chain CDR1 comprises amino acid sequence of SEQ ID No.102, the light chain CDR2 comprises amino acid sequence of SEQ ID No.103 and the light chain CDR3 comprises amino acid sequence of SEQ ID No.104. Preferably, aforesaid protein comprises the variable region of BLyS antibody heavy chain and/or variable region of BLyS antibody light chain forming by CDR and framework, the amino acid sequences of heavy chain variable region are represented by SEQ ID No.1, SEQ ID No.9, SEQ ID No.17, SEQ ID No.25, SEQ ID No.33, SEQ ID No.41, SEQ ID No.49, SEQ ID No.57, SEQ ID No.65, SEQ ID No.73, SEQ ID No.81, SEQ ID No.89 or SEQ ID No.97; the amino acid sequences of light chain variable region are represented by SEQ ID No.5, SEQ ID No.13, SEQ ID No.21, SEQ ID No.29, SEQ ID No.37, SEQ ID No.45, SEQ ID No.53, SEQ ID No.61, SEQ ID No.69, SEQ ID No.77, SEQ ID No.85, SEQ ID No.93 or SEQ ID No.101.

The invention further provides an isolated protein, comprising a variable region of BLyS antibody heavy chain and/or variable region of BLyS antibody light chain, the amino acid sequences of heavy chain variable region are represented by SEQ ID No.1, SEQ ID No.9, SEQ ID No.17, SEQ ID No.25, SEQ ID No.33, SEQ ID No.41, SEQ ID No.49, SEQ ID No.57, SEQ ID No.65, SEQ ID No.73, SEQ ID No.81, SEQ ID No.89 or SEQ ID No.97; the amino acid sequences of light chain variable region are represented by SEQ ID No.5, SEQ ID No.13, SEQ ID No.21, SEQ ID No.29, SEQ ID No.37, SEQ ID No.45, SEQ ID No.53, SEQ ID No.61, SEQ ID No.69, SEQ ID No.77, SEQ ID No.85, SEQ ID No.93 or SEQ ID No.101.

Preferably, an amino acid sequence of the heavy chain variable region is shown in SEQ ID No.1 and an amino acid sequence of the light chain variable region is shown in SEQ ID No.5; an amino acid sequence of the heavy chain variable region is shown in SEQ ID No.9 and an amino acid sequence of the light chain variable region is shown in SEQ ID No.13; an amino acid sequence of the heavy chain variable region is shown in SEQ ID No.17 and an amino acid sequence of the light chain variable region is shown in SEQ ID No.21; an amino acid sequence of the heavy chain variable region is shown in SEQ ID No.25 and an amino acid sequence of the light chain variable region is shown in SEQ ID No.29; an amino acid sequence of the heavy chain variable region is shown in SEQ ID No.33 and an amino acid sequence of the light chain variable region is shown in SEQ ID No.37; an amino acid sequence of the heavy chain variable region is shown in SEQ ID No.41 and an amino acid sequence of the light chain variable region is shown in SEQ ID No.45; an amino acid sequence of the heavy chain variable region is shown in SEQ ID No.49 and an amino acid sequence of the light chain variable region is shown in SEQ ID No.53; an amino acid sequence of the heavy chain variable region is shown in SEQ ID No.57 and an amino acid sequence of the light chain variable region is shown in SEQ ID No.61; an amino acid sequence of the heavy chain variable region is shown in SEQ ID No.65 and an amino acid sequence of the light chain variable region is shown in SEQ ID No.69; an amino acid sequence of the heavy chain variable region is shown in SEQ ID No.73 and an amino acid sequence of the light chain variable region is shown in SEQ ID No.77; an amino acid sequence of the heavy chain variable region is shown in SEQ ID No.81 and an amino acid sequence of the light chain variable region is shown in SEQ ID No.85; an amino acid sequence of the heavy chain variable region is shown in SEQ ID No.89 and an amino acid sequence of the light chain variable region is shown in SEQ ID No.93; or, an amino acid sequence of the heavy chain variable region is shown in SEQ ID No.97 and an amino acid sequence of the light chain variable region is shown in SEQ ID No.101.

In this art, antibody-antigen binding domain each contains a light chain variable region and a heavy chain variable region, meanwhile each variable region contains three domains, CDR1, CDR2 and CDR3.

In summary, the numbers of the above-mentioned amino acid sequences are shown in Table 1:

TABLE 1
BLyS Antibody Amino Acid SEQ ID NO.

| | Heavy Chain | | | | Light Chain | | | |
|---|---|---|---|---|---|---|---|---|
| | Variable | | | | Variable | | | |
| Clone No. | Region | CDR1 | CDR2 | CDR3 | Region | CDR1 | CDR2 | CDR3 |
| 2-1G11 | 1 | 2 | 3 | 4 | 5 | 6 | 7 | 8 |
| L9G7 | 9 | 10 | 11 | 12 | 13 | 14 | 15 | 16 |
| L1D12 | 17 | 18 | 19 | 20 | 21 | 22 | 23 | 24 |
| 35E6F7C3 | 25 | 26 | 27 | 28 | 29 | 30 | 31 | 32 |
| 8E7D9C7F5 | 33 | 34 | 35 | 36 | 37 | 38 | 39 | 40 |
| 20D1B6E9E5 | 41 | 42 | 43 | 44 | 45 | 46 | 47 | 48 |
| 78C11D2D12 | 49 | 50 | 51 | 52 | 53 | 54 | 55 | 56 |
| 89A2G5E7 | 57 | 58 | 59 | 60 | 61 | 62 | 63 | 64 |
| 97E7B3F2 | 65 | 66 | 67 | 68 | 69 | 70 | 71 | 72 |
| 97A3C2H4 | 73 | 74 | 75 | 76 | 77 | 78 | 79 | 80 |
| 67A2E1D10 | 81 | 82 | 83 | 84 | 85 | 86 | 87 | 88 |
| 111D10D6G3 | 89 | 90 | 91 | 92 | 93 | 94 | 95 | 96 |
| 93C6F10D3 | 97 | 98 | 99 | 100 | 101 | 102 | 103 | 104 |

Where the numbers in Table 1 are the sequence numbers in the sequence listing, for example, the amino acid sequence of heavy chain variable region of 2-1G11 is shown in SEQ ID No. 1, and the amino acid sequence of heavy chain CDR1 of 2-1G11 is shown in SEQ ID No. 2.

The isolated protein in the present invention further comprises a framework region (or framework or FR) of BLyS antibody, and the framework region comprises a framework region of heavy chain and/or a framework region of light chain; and preferably, the framework region of heavy chain is a framework region of human or mouse antibody heavy chain, and/or the framework region of light chain is a framework region of human or mouse antibody light chain.

More preferably, when the isolated protein in the present invention is humanized antibody or fully human antibody, the framework region of heavy chain is a framework region of human antibody heavy chain, and the framework residues of human antibody heavy chain may contain germlines DP4, DP7, DP8, DP9, DP10, DP14(V_{H}1-18), DP31, DP33, DP35(V_{H}3-11), DP45, DP46, DP47, DP48, DP49(V_{H}3-30), DP50, DP51(V_{H}3-48), DP53, DP54(V_{H}3-7), DP65, DP66, DP67, DP68 and DP69, especially the FR1, FR2, FR3 of these germlines; as well as JH fragments J_{H}-1, J_{H}-2, J_{H}-3, J_{H}-4, J_{H}-4b, J_{H}-5 and J_{H}-6, especially the sequences encoded by FR4 of these germlines, or the consensus sequences of the heavy chain framework regions. The framework region of light chain is a framework region of human antibody light chain, and the framework residues of human antibody light chain may contain germlines O2, O12, DPK1(O18), DPK2, DPK3, DPK4, DPK5, DPK6, DPK7, DPK8, DPK9, DPK10, DPK12(A2), DPK13, DPK15, DPK16, DPKI8, DPK19, DPK20, DPK21, DPK22, DPK23, DPK24(B3), DPK25, DPK26(A10) and DPK 28, especially the FR1, FR2, FR3 of these germlines; as well as Jk fragments Jk1, Jk2, Jk3, Jk4 and Jk5, especially the sequences encoded by FR4 of these germlines. Such sequences of framework regions may be obtained from public DNA databases including germline antibody gene sequences or published references. For example, germline DNA sequences of variable region genes of human heavy and light chain are available in the “VBase” human germline sequence database (http://www2.mrc-lmb.cam.ac.uk/vbase/) and the Kabat (E. A et al., 1991 Sequences of Proteins of Immunological Interest, 5th edition). In a better embodiment of the humanized antibody of the present invention, the framework residues of heavy chain is preferably V_{H}1-18 of V_{H} exon, V_{H}3-7 of V_{H} exon or J_{H}-6 of J_{H} exon from a heavy chain of human germline antibody, and the framework residues of light chain is preferably B3 of V_{K} exon, J_{K}-4 of the J_{K} exon or A10 of the V_{K} exon from a light chain of human germline antibody.

Preferably, the protein further comprises heavy chain constant region of antibody and/or light chain constant region of antibody, wherein the heavy chain constant region of antibody is known in the art, preferably is a heavy chain constant region of mouse antibody or a heavy chain constant region of human antibody, more preferably is a heavy chain constant region of human antibody. The light chain constant region of antibody is known in the art, preferably is a light chain constant region of mouse antibody or a light chain constant region of human antibody, more preferably is a light chain constant region of human antibody.

Where the mouse heavy chain constant region and mouse light chain constant region, or human heavy chain constant region and human light chain constant region can form murine BLyS antibody or BLyS chimeric antibody with the heavy chain variable regions having amino acid sequence shown in SEQ ID NO.25, 33, 41, 49, 57, 65, 73, 81, 89 or 97 and the light chain variable regions having amino acid sequence shown in SEQ ID NO. 29, 37, 45, 53, 61, 69, 77, 85, 93 or 101, respectively.

Further, the heavy chain CDRs sequences shown in SEQ ID NO. 26-28, 34-36, 42-44, 50-52, 58-60, 66-68, 74-76, 82-84, 90-92, or 98-100 determined by the amino acid sequence of the heavy chain variable region of SEQ ID NO. 25, 33, 41, 49, 57, 65, 73, 81, 89 or 97 according to Kabat definition in aforementioned chimeric antibody, and the light chain CDRs sequences of SEQ ID NO. 30-32, 38-40, 46-48, 54-56, 62-64, 70-72, 78-80, 86-88, 94-96 or 102-104 determined by aforementioned amino acid sequence of the light chain variable region of SEQ ID NO. 29, 37, 45, 53, 61, 69, 77, 85, 93 or 101 according to Kabat definition, were transplanted into the selected templates of human germline to replace the CDR region in the templates of human germline and then the humanized antibodies were constructed, respectively. The light chain framework region and heavy chain framework region in the germline templates are as described above, preferably selecting from V_{H}1-18 of V_{H} exon, V_{H}3-7 of V_{H} exon or J_{H}-6 of J_{H} exon in a heavy chain of human germline antibody, and B3 of V_{K} exon, J_{K}-4 of the J_{K} exon or A10 of the V_{K} exon in a light chain of human germline antibody. Optionally, in order to ensure the activity of antibodies, the buried residues, the residues that have direct interaction with the CDR regions, and the framework residues that have a significant influence on the conformations of VH and VL were back-mutated based on the three-dimensional structure of mouse antibody.

Preferably, the amino sequences of the heavy chain variable region, in which its CDR regions were transplanted into the selected human germline templates and the residues of framework regions were back-mutated, preferably are shown in SEQ ID NO. 140, SEQ ID NO. 141, SEQ ID NO. 142, SEQ ID NO. 143, SEQ ID NO. 144, SEQ ID NO. 145, SEQ ID NO. 146, SEQ ID NO. 149, SEQ ID NO. 150, SEQ ID NO. 151 or SEQ ID NO. 152. The amino acid sequences of the light chain variable region preferably are shown in SEQ ID No. 147, SEQ ID No. 148, SEQ ID No. 153, SEQ ID No. 154, SEQ ID No. 155, SEQ ID No. 156 or SEQ ID No.157.

Sequence of the heavy chain variable region shown in SEQ ID NO. 1, 9 or 17 and sequence of the light chain variable region shown in SEQ ID NO. 5, 13 or 21 can form human BLyS antibody with constant region of humanized antibody heavy chain as well as constant region of humanized antibody light chain.

The protein is antibody protein, preferably one or more of full-length antibody protein, antibody-antigen binding domain protein fragment, bispecific antibody, multispecific antibody, single-chain antibody fragment (scFv), single-domain antibody (sdAb) or single-region antibody, as well as the monoclonal antibody or polyclonal antibody produced by aforesaid antibodies. The monoclonal antibody can be developed using a variety of routes and technologies such as hybridoma technology, phage display technology, single lymphocyte gene cloning technology and the like, and the production of monoclonal antibody from wild-type or transgenic mice by hybridoma technology is current mainstream technology.

The full-length antibody protein is a conventional full-length antibody protein known in the art, which comprises heavy chain variable region, light chain variable region, heavy chain constant region and light chain constant region. The heavy chain variable region and light chain variable region of the protein form human full-length antibody protein with human heavy chain constant region and human light chain constant region. Preferably, the full-length antibody protein is IgG1, IgG2, IgG3 or IgG4.

The single-chain antibody is a conventional single-chain antibody known in the art, which comprises heavy chain variable region, light chain variable region and short peptide of 15-20 amino acids.

The protein fragment of antibody-antigen binding domain is a conventional protein fragment of antibody-antigen binding domain known in the art, which comprises light chain variable region, light chain constant region and Fd fragment of heavy chain constant region. Preferably, the protein fragment of antibody-antigen binding domain is Fab and F(ab′)2.

The single-domain antibody is a conventional single-domain antibody known in the art, which comprises heavy chain variable region and heavy chain constant region.

The single-region antibody is a conventional single-region antibody known in the art, which only comprises heavy chain variable region.

The preparation method of the protein is a conventional preparation method known in the art. The preparation method is preferably: obtain the protein by isolating the protein from an expression transformant that recombinantly expresses the protein or by artificial synthesis of protein sequences. Preferably, the method for isolating the protein from the expression transformant that recombinantly expresses the protein includes: cloning a nucleic acid that encodes a protein and comprises a point mutation into a recombinant vector, transforming the recombinant vector into the transformant to obtain a recombinant expression transformant, and isolating and purifying the protein from the culture of the recombinant expression transformant.

The present invention further provides a nucleic acid encoding aforesaid protein.

Preferably, the nucleotide sequences of the nucleic acid encoding the heavy chain variable region are shown in SEQ ID No.105, SEQ ID No.107, SEQ ID No.109, SEQ ID No.111, SEQ ID No.113, SEQ ID No.115, SEQ ID No.117, SEQ ID No.119, SEQ ID No.121, SEQ ID No.123, SEQ ID No.125, SEQ ID No.127 or SEQ ID No.129; and/or, the nucleotide sequences of the nucleic acid encoding the light chain variable region are shown in SEQ ID No.106, SEQ ID No.108, SEQ ID No.110, SEQ ID No.112, SEQ ID No.114, SEQ ID No.116, SEQ ID No.118, SEQ ID No.120, SEQ ID No.122, SEQ ID No.124, SEQ ID No.126, SEQ ID No.128 or SEQ ID No.130.

More preferably, a nucleotide sequence of the nucleic acid encoding the heavy chain variable region is shown in SEQ ID No.105, and a nucleotide sequence of the nucleic acid encoding the light chain variable region is shown in SEQ ID No.106; a nucleotide sequence of the nucleic acid encoding the heavy chain variable region is shown in SEQ ID No.107, and a nucleotide sequence of the nucleic acid encoding the light chain variable region is shown in SEQ ID No.108; a nucleotide sequence of the nucleic acid encoding the heavy chain variable region is shown in SEQ ID No.109, and a nucleotide sequence of the nucleic acid encoding the light chain variable region is shown in SEQ ID No.110; a nucleotide sequence of the nucleic acid encoding the heavy chain variable region is shown in SEQ ID No.111, and a nucleotide sequence of the nucleic acid encoding the light chain variable region is shown in SEQ ID No.112; a nucleotide sequence of the nucleic acid encoding the heavy chain variable region is shown in SEQ ID No.113, and a nucleotide sequence of the nucleic acid encoding the light chain variable region is shown in SEQ ID No.114; a nucleotide sequence of the nucleic acid encoding the heavy chain variable region is shown in SEQ ID No.115, and a nucleotide sequence of the nucleic acid encoding the light chain variable region is shown in SEQ ID No.116; a nucleotide sequence of the nucleic acid encoding the heavy chain variable region is shown in SEQ ID No.117, and a nucleotide sequence of the nucleic acid encoding the light chain variable region is shown in SEQ ID No.118; a nucleotide sequence of the nucleic acid encoding the heavy chain variable region is shown in SEQ ID No.119, and a nucleotide sequence of the nucleic acid encoding the light chain variable region is shown in SEQ ID No.120; a nucleotide sequence of the nucleic acid encoding the heavy chain variable region is shown in SEQ ID No.121, and a nucleotide sequence of the nucleic acid encoding the light chain variable region is shown in SEQ ID No.122; a nucleotide sequence of the nucleic acid encoding the heavy chain variable region is shown in SEQ ID No.123, and a nucleotide sequence of the nucleic acid encoding the light chain variable region is shown in SEQ ID No.124; a nucleotide sequence of the nucleic acid encoding the heavy chain variable region is shown in SEQ ID No.125, and a nucleotide sequence of the nucleic acid encoding the light chain variable region is shown in SEQ ID No.126; a nucleotide sequence of the nucleic acid encoding the heavy chain variable region is shown in SEQ ID No.127, and a nucleotide sequence of the nucleic acid encoding the light chain variable region is shown in SEQ ID No.128; a nucleotide sequence of the nucleic acid encoding the heavy chain variable region is shown in SEQ ID No.129, and a nucleotide sequence of the nucleic acid encoding the light chain variable region is shown in SEQ ID No.130.

In summary, the sequence ID numbers of the aforementioned nucleotide sequences are shown in Table 2:

TABLE 2
Nucleotide SEQ ID NO of BLyS Antibody Gene.

| | Heavy chain | Light chain |
|---|---|---|
| Clone No. | variable region | variable region |
| 2-1G11 | 105 | 106 |
| L9G7 | 107 | 108 |
| L1D12 | 109 | 110 |
| 35E6F7C3 | 111 | 112 |
| 8E7D9C7F5 | 113 | 114 |
| 20D1B6E9E5 | 115 | 116 |
| 78C11D2D12 | 117 | 118 |
| 89A2G5E7 | 119 | 120 |
| 97E7B3F2 | 121 | 122 |
| 97A3C2H4 | 123 | 124 |
| 67A2E1D10 | 125 | 126 |
| 111D10D6G3 | 127 | 128 |
| 93C6F10D3 | 129 | 130 |

As used herein, the numbers in Table 2 are the sequence ID numbers in the Sequence Listing, for example, the nucleotide sequence of the amino acid encoding the heavy chain variable region of 2-1G11 is shown in SEQ ID No.105, and the nucleotide sequence of the amino acid encoding the light chain variable region of 2-1G11 is shown in SEQ ID No.106.

The nucleotide sequence encoding the heavy chain CDR1 of 2-1G11 is the sequence from 76th to 105th base shown in SEQ ID No.105 of the Sequence Listing.

The nucleotide sequence encoding the heavy chain CDR2 of 2-1G11 is the sequence from 148th to 198th base shown in SEQ ID No.105 of the Sequence Listing.

The nucleotide sequence encoding the heavy chain CDR3 of 2-1G11 is the sequence from 295th to 342nd base shown in SEQ ID No.105 of the Sequence Listing.

The nucleotide sequence encoding the light chain CDR1 of 2-1G11 is the sequence from 70th to 102nd base shown in SEQ ID No.106 of the Sequence Listing.

The nucleotide sequence encoding the light chain CDR2 of 2-1G11 is the sequence from 148th to 168th base shown in SEQ ID No.106 of the Sequence Listing.

The nucleotide sequence encoding the light chain CDR3 of 2-1G11 is the sequence from 265th to 291th base shown in SEQ ID No.106 of the Sequence Listing.

The nucleotide sequence encoding the heavy chain CDR1 of L9G7 is the sequence from 76th to 105th base shown in SEQ ID No.107 of the Sequence Listing.

The nucleotide sequence encoding the heavy chain CDR2 of L9G7 is the sequence from 148th to 198th base shown in SEQ ID No.107 of the Sequence Listing.

The nucleotide sequence encoding the heavy chain CDR3 of L9G7 is the sequence from 295th to 342nd base shown in SEQ ID No.107 of the Sequence Listing.

The nucleotide sequence encoding the light chain CDR1 of L9G7 is the sequence from 70th to 102nd base shown in SEQ ID No.108 of the Sequence Listing.

The nucleotide sequence encoding the light chain CDR2 of L9G7 is the sequence from 148th to 168th base shown in SEQ ID No.108 of the Sequence Listing.

The nucleotide sequence encoding the light chain CDR3 of L9G7 is the sequence from 265th to 291nd base shown in SEQ ID No.108 of the Sequence Listing.

The nucleotide sequence encoding the heavy chain CDR1 of L1D12 is the sequence from 76th to 105th base shown in SEQ ID No.109 of the Sequence Listing.

The nucleotide sequence encoding the heavy chain CDR2 of L1D12 is the sequence from 148th to 195th base shown in SEQ ID No.109 of the Sequence Listing.

The nucleotide sequence encoding the heavy chain CDR3 of L1D12 is the sequence from 292nd to 330nd base shown in SEQ ID No.109 of the Sequence Listing.

The nucleotide sequence encoding the light chain CDR1 of L1D12 is the sequence from 67th to 99th base shown in SEQ ID No.110 of the Sequence Listing.

The nucleotide sequence encoding the light chain CDR2 of L1D12 is the sequence from 145th to 165th base shown in SEQ ID No.110 of the Sequence Listing.

The nucleotide sequence encoding the light chain CDR3 of L1D12 is the sequence from 262nd to 297th base shown in SEQ ID No.110 of the Sequence Listing.

The nucleotide sequence encoding the heavy chain CDR1 of 35E6F7C3 is the sequence from 76th to 105th base shown in SEQ ID No.111 of the Sequence Listing.

The nucleotide sequence encoding the heavy chain CDR2 of 35E6F7C3 is the sequence from 148th to 198th base shown in SEQ ID No.111.

The nucleotide sequence encoding the heavy chain CDR3 of 35E6F7C3 is the sequence from 295nd to 321th base shown in SEQ ID No.111 of the Sequence Listing.

The nucleotide sequence encoding the light chain CDR1 of 35E6F7C3 is the sequence from 70th to 99th base shown in SEQ ID No.112 of the Sequence Listing.

The nucleotide sequence encoding the light chain CDR2 of 35E6F7C3 is the sequence from 145th to 165th base shown in SEQ ID No.112 of the Sequence Listing.

The nucleotide sequence encoding the light chain CDR3 of 35E6F7C3 is the sequence from 262nd to 288th base shown in SEQ ID No.112 of the Sequence Listing.

The nucleotide sequence encoding the heavy chain CDR1 of 8E7D9C7F5 is the sequence from 76th to 105th base shown in SEQ ID No.113 of the Sequence Listing.

The nucleotide sequence encoding the heavy chain CDR2 of 8E7D9C7F5 is the sequence from 148th to 198th base shown in SEQ ID No.113 of the Sequence Listing.

The nucleotide sequence encoding the heavy chain CDR3 of 8E7D9C7F5 is the sequence from 295nd to 342nd base shown in SEQ ID No.113 of the Sequence Listing.

The nucleotide sequence encoding the light chain CDR1 of 8E7D9C7F5 is the sequence from 70th to 114th base shown in SEQ ID No.114 of the Sequence Listing.

The nucleotide sequence encoding the light chain CDR2 of 8E7D9C7F5 is the sequence from 160th to 180th base shown in SEQ ID No.114 of the Sequence Listing.

The nucleotide sequence encoding the h light chain CDR3 of 8E7D9C7F5 is the sequence from 277nd to 303th base shown in SEQ ID No.114 of the Sequence Listing.

The nucleotide sequence encoding the heavy chain CDR1 of 20D1B6E9E5 is the sequence from 76th to 105th base shown in SEQ ID No.115 of the Sequence Listing.

The nucleotide sequence encoding the heavy chain CDR2 of 20D1B6E9E5 is the sequence from 148th to 198th base shown in SEQ ID No.115 of the Sequence Listing.

The nucleotide sequence encoding the heavy chain CDR3 of 20D1B6E9E5 is the sequence from 295nd to 339th base shown in SEQ ID No.115 of the Sequence Listing.

The nucleotide sequence encoding the light chain CDR1 of 20D1B6E9E5 is the sequence from 70th to 117th base shown in SEQ ID No.116 of the Sequence Listing.

The nucleotide sequence encoding the light chain CDR2 of 20D1B6E9E5 is the sequence from 163th to 183th base shown in SEQ ID No.116 of the Sequence Listing.

The nucleotide sequence encoding the light chain CDR3 of 20D1B6E9E5 is the sequence from 280nd to 306th base shown in SEQ ID No.116 of the Sequence Listing.

The nucleotide sequence encoding the heavy chain CDR1 of 78C11D2D12 is the sequence from 76th to 105th base shown in SEQ ID No.117 of the Sequence Listing.

The nucleotide sequence encoding the heavy chain CDR2 of 78C11D2D12 is the sequence from 148th to 198th base shown in SEQ ID No.117 of the Sequence Listing.

The nucleotide sequence encoding the heavy chain CDR3 of 78C11D2D12 is the sequence from 295nd to 315th base shown in SEQ ID No.117 of the Sequence Listing.

The nucleotide sequence encoding the light chain CDR1 of 78C11D2D12 is the sequence from 70th to 99th base shown in SEQ ID No.118 of the Sequence Listing.

The nucleotide sequence encoding the light chain CDR2 of 78C11D2D12 is the sequence from 145th to 165th base shown in SEQ ID No.118 of the Sequence Listing.

The nucleotide sequence encoding the light chain CDR3 of 78C11D2D12 is the sequence from 262nd to 288th base shown in SEQ ID No.118 of the Sequence Listing.

The nucleotide sequence encoding the heavy chain CDR1 of 89A2G5E7 is the sequence from 76th to 105th base shown in SEQ ID No.119 of the Sequence Listing.

The nucleotide sequence encoding the heavy chain CDR2 of 89A2G5E7 is the sequence from 148th to 195th base shown in SEQ ID No.119 of the Sequence Listing.

The nucleotide sequence encoding the heavy chain CDR3 of 89A2G5E7 is the sequence from 292nd to 327th base shown in SEQ ID No.119 of the Sequence Listing.

The nucleotide sequence encoding the light chain CDR1 of 89A2G5E7 is the sequence from 70th to 105th base shown in SEQ ID No.120 of the Sequence Listing.

The nucleotide sequence encoding the light chain CDR2 of 89A2G5E7 is the sequence from 151th to 171th base shown in SEQ ID No.120 of the Sequence Listing.

The nucleotide sequence encoding the light chain CDR3 of 89A2G5E7 is the sequence from 268nd to 294th base shown in SEQ ID No.120 of the Sequence Listing.

The nucleotide sequence encoding the heavy chain CDR1 of 97E7B3F2 is the sequence from 76th to 105th base shown in SEQ ID No.121 of the Sequence Listing.

The nucleotide sequence encoding the heavy chain CDR2 of 97E7B3F2 is the sequence from 148th to 198th base shown in SEQ ID No.121 of the Sequence Listing.

The nucleotide sequence encoding the heavy chain CDR3 of 97E7B3F2 is the sequence from 295nd to 321th base shown in SEQ ID No.121 of the Sequence Listing.

The nucleotide sequence encoding the light chain CDR1 of 97E7B3F2 is the sequence from 70th to 117th base shown in SEQ ID No.122 of the Sequence Listing.

The nucleotide sequence encoding the light chain CDR2 of 97E7B3F2 is the sequence from 163th to 183th base shown in SEQ ID No.122 of the Sequence Listing.

The nucleotide sequence encoding the light chain CDR3 of 97E7B3F2 is the sequence from 280nd to 306th base shown in SEQ ID No.122 of the Sequence Listing.

The nucleotide sequence encoding the heavy chain CDR1 of 97A3C2H4 is the sequence from 76th to 105th base shown in SEQ ID No.123 of the Sequence Listing.

The nucleotide sequence encoding the heavy chain CDR2 of 97A3C2H4 is the sequence from 148th to 198th base shown in SEQ ID No.123 of the Sequence Listing.

The nucleotide sequence encoding the heavy chain CDR3 of 97A3C2H4 is the sequence from 295nd to 327th base shown in SEQ ID No.123 of the Sequence Listing.

The nucleotide sequence encoding the light chain CDR1 of 97A3C2H4 is the sequence from 70th to 102th base shown in SEQ ID No.124 of the Sequence Listing.

The nucleotide sequence encoding the light chain CDR2 of 97A3C2H4 is the sequence from 148th to 168th base shown in SEQ ID No.124 of the Sequence Listing.

The nucleotide sequence encoding the light chain CDR3 of 97A3C2H4 is the sequence from 265nd to 291th base shown in SEQ ID No.124 of the Sequence Listing.

The nucleotide sequence encoding the heavy chain CDR1 of 67A2E1D10 is the sequence from 76th to 105th base shown in SEQ ID No.125 of the Sequence Listing.

The nucleotide sequence encoding the heavy chain CDR2 of 67A2E1D10 is the sequence from 148th to 198th base shown in SEQ ID No.125 of the Sequence Listing.

The nucleotide sequence encoding the heavy chain CDR3 of 67A2E1D10 is the sequence from 295nd to 333th base shown in SEQ ID No.125 of the Sequence Listing.

The nucleotide sequence encoding the light chain CDR1 of 67A2E1D10 is the sequence from 70th to 102nd base shown in SEQ ID No.126 of the Sequence Listing.

The nucleotide sequence encoding the light chain CDR2 of 67A2E1D10 is the sequence from 148th to 168th base shown in SEQ ID No.126 of the Sequence Listing.

The nucleotide sequence encoding the light chain CDR3 of 67A2E1D10 is the sequence from 265nd to 291th base shown in SEQ ID No.126 of the Sequence Listing.

The nucleotide sequence encoding the heavy chain CDR1 of 111D10D6G3 is the sequence from 76th to 105th base shown in SEQ ID No.127 of the Sequence Listing.

The nucleotide sequence encoding the heavy chain CDR2 of 111D10D6G3 is the sequence from 148th to 198th base shown in SEQ ID No.127 of the Sequence Listing.

The nucleotide sequence encoding the heavy chain CDR3 of 111D10D6G3 is the sequence from 295nd to 309th base shown in SEQ ID No.127 of the Sequence Listing.

The nucleotide sequence encoding the light chain CDR1 of 111D10D6G3 is the sequence from 70th to 102th base shown in SEQ ID No.128 of the Sequence Listing.

The nucleotide sequence encoding the light chain CDR2 of 111D10D6G3 is the sequence from 148th to 168th base shown in SEQ ID No.128 of the Sequence Listing.

The nucleotide sequence encoding the light chain CDR3 of 111D10D6G3 is the sequence from 265nd to 291th base shown in SEQ ID No.128 of the Sequence Listing.

The nucleotide sequence encoding the heavy chain CDR1 of 93C6F10D3 is the sequence from 76th to 105th base shown in SEQ ID No.129 of the Sequence Listing.

The nucleotide sequence encoding the heavy chain CDR2 of 93C6F10D3 is the sequence from 148th to 198th base shown in SEQ ID No.129 of the Sequence Listing.

The nucleotide sequence encoding the heavy chain CDR3 of 93C6F10D3 is the sequence from 295nd to 336th base shown in SEQ ID No.129 of the Sequence Listing.

The nucleotide sequence encoding the light chain CDR1 of 93C6F10D3 is the sequence from 70th to 102th base shown in SEQ ID No.130 of the Sequence Listing.

The nucleotide sequence encoding the light chain CDR2 of 93C6F10D3 is the sequence from 148th to 168th base shown in SEQ ID No.130 of the Sequence Listing.

The nucleotide sequence encoding the light chain CDR3 of 93C6F10D3 is the sequence from 265nd to 291th base shown in SEQ ID No.130 of the Sequence Listing.

In a preferable embodiment of the present invention, the nucleotide sequence of the nucleic acid encoding the heavy chain variable region of the humanized antibody of the present invention, which is derived from the human framework region, is shown in SEQ ID NO. 158, SEQ ID NO. 159, SEQ ID NO. 160, SEQ ID NO. 161, SEQ ID NO. 162, SEQ ID NO. 163, SEQ ID NO. 164, SEQ ID NO. 167, SEQ ID NO. 168, SEQ ID NO. 169 or SEQ ID NO. 170 of the Sequencing Listing; and/or, the nucleotide sequence of the nucleic acid encoding the light chain variable region is shown in SEQ ID No.165, SEQ ID No.166, SEQ ID No.171, SEQ ID No.172, SEQ ID No.173, SEQ ID No.174, or SEQ ID No.175 of the Sequencing Listing.

The preparation method of the nucleic acid is a conventional preparation method known in the art, preferably comprising following steps: the nucleic acid molecules encoding aforesaid proteins are obtained by gene cloning technology, or the nucleic acid molecules encoding aforesaid proteins are obtained by the method of artificial full sequence synthesis.

Person skilled in the art knows that the substitution, deletion, alteration, insertion or addition can be introduced into the base sequence encoding the amino acid sequence of aforesaid protein as appropriate to provide a homologue of polynucleotide. A homologue of polynucleotide in the present invention can be prepared by substituting, deleting or adding one or more bases of a gene encoding the protein sequence while the activity of the antibody is maintained.

The present invention further provides a recombinant expression vector comprising the nucleic acid.

As used herein, the recombinant expression vector can be obtained by the conventional method known in the art, that is, constructing the nucleic acid molecule of the present invention to various expression vectors. The expression vectors are a variety of vectors that are conventional in the art, as long as the vectors can carry aforesaid nucleic acid molecule. The vectors preferably include various plasmids, cosmids, phages or viral vectors.

The present invention further provides a recombinant expression transformant comprising the recombinant expression vector.

As used herein, the method for preparing the recombinant expression transformant is a conventional preparation method known in the art, preferably transforming the recombinant expression vector into host cells. The host cells are conventional various host cells known in the art, as long as they are able to stably self-replicate aforesaid recombinant expression vectors and efficiently express the carried nucleic acid.

Preferably, the host cells are E. coli TG1 or E. coli BL21 cells (expressing single chain antibody or Fab antibody), or HEK293 or CHO-K1 cells (expressing full-length IgG antibody). The preferred recombinant expression transformant of the present invention can be obtained by transforming aforesaid recombinant expression plasmids into host cells. As used herein, the transformation method is a conventional method known in the art, preferably chemical transformation method, heat shock method or electrotransformation method.

The present invention further provides a method for preparing a BLyS antibody, which comprises following steps: culturing the recombinant expression transformant, and obtaining BLyS antibody from the culture.

The present invention further provides a method for detecting cells that overexpressing BLyS protein, which comprises following steps: contacting the protein with a sample to be tested in vitro, and detecting the combination between the protein and the sample to be tested.

The definition of overexpression is conventional in the art, which refers to the overexpression of RNA or protein of BLyS in the sample to be tested (due to increased transcription, post-transcriptional processing, translation, post-translational processing and the alternation in protein degradation), and local over-expression and increased functional activity (e.g. in the case of increased enzymatic hydrolysis of the substrate) resulting from changes in protein transporting patterns (increased nuclear localization).

As used herein, the method for detecting aforesaid combination is the conventional method known in the art, preferably the detection by fluorescence activated cell sorter (FACS).

The present invention provides a composition for detecting cells that overexpress BLyS protein, which comprises aforesaid protein as an active ingredient. Preferably, it further comprises compound consisting of functional fragments of aforesaid protein as an active ingredient.

The present invention provides a use of aforesaid protein in the preparation of medicament.

Preferably, the medicament is a medicament for preventing or treating diseases associated with abnormal expression or dysfunction of BLyS.

As used herein, the diseases associated with abnormal expression or dysfunction of BLyS is conventional diseases associated with abnormal expression or dysfunction of BLyS known in the art. Preferably autoimmune diseases, inflammatory diseases, infectious diseases or proliferative diseases. In the present invention, the autoimmune diseases are conventional autoimmune diseases known in the art, preferably systemic lupus erythematosus (SLE), rheumatoid arthritis (RA), Sjogren's syndrome (SS), multiple sclerosis (MS), myasthenia gravis (MG), chronic thyroiditis or immunodeficiency syndrome. In the present invention, the inflammatory diseases are conventional inflammatory diseases known in the art, preferably asthma or allergic diseases. In the present invention, the infectious diseases are conventional infectious diseases known in the art. Preferably acquired immune deficiency syndrome (AIDS).

In the present invention, the proliferative diseases are conventional proliferative diseases known in the art, preferably leukemia, tumor or lymphoma.

The present invention further provides a pharmaceutical composition, comprising aforesaid protein as an active ingredient.

Preferably, the pharmaceutical composition is a pharmaceutical composition for preventing or treating diseases associated with abnormal expression or dysfunction of BLyS.

The administration route of the pharmaceutical composition in the present invention is preferably administered by injection or administered orally. The injection administration preferably includes intravenous injection, intramuscular injection, intraperitoneal injection, intradermal injection or subcutaneous injection. The pharmaceutical composition is in the form of various forms conventionally known in the art, preferably in solid, semisolid or liquid form, and may be aqueous solutions, non-aqueous solutions or suspensions, more preferably tablets, capsules, granules, injections or infusions and the like.

As used herein, preferably, the pharmaceutical composition of the present invention further comprises one or more pharmaceutical carriers. The pharmaceutical carrier is the conventional pharmaceutical carrier known in the art, and the pharmaceutical carrier can be any suitable physiologically or pharmaceutically acceptable pharmaceutical adjuvant. The pharmaceutical adjuvant is t conventional pharmaceutical adjuvant known in the art, preferably includes pharmaceutically acceptable excipients, fillers, diluents and the like. More preferably, the pharmaceutical composition comprises 0.01-99.99% of aforesaid protein and 0.01-99.99% of the pharmaceutical carrier, and the percentage is the mass percentage of the pharmaceutical composition.

As used herein, preferably, the pharmaceutical composition is administered in an amount effective to alleviate or delay the progression of a disease, degenerative or traumatic disorder. The effective amount can be determined on an individual basis and will be based in part on the consideration of the symptoms to be treated and the outcome sought. The effective amount can be determined by person skilled in the art using aforesaid factors such as individual difference of subjects and conventional experimentation.

The present invention provides the use of aforesaid protein in preparing medicament for preventing or treating diseases associated with abnormal expression or dysfunction of BLyS; preferably, the diseases associated with abnormal expression or dysfunction of BLyS are autoimmune diseases, inflammatory diseases, infectious diseases or proliferative diseases.

The present invention provides the use of aforesaid pharmaceutical composition in preparing drugs for preventing or treating diseases associated with abnormal expression or dysfunction of BLyS; preferably, the diseases associated with abnormal expression or dysfunction of BLyS are autoimmune diseases, inflammatory diseases, infectious diseases or proliferative diseases.

Based on the common knowledge in the art, aforesaid preferred conditions can be combined arbitrarily to obtain the preferable embodiments of the present invention.

The reagents and raw materials used in the present invention are commercially available.

The advantage of the present invention is that the protein of the present invention is a humanized or human BLyS antibody, which has a high affinity for BLyS protein (affinity KD<5×10⁻⁹M), can significantly block BLyS activities at the protein level and cellular level, and prevent the binding of BLyS to its receptors. The BLyS antibody lacks a cross-reaction with homologous protein antigen such as human APRIL. It was shown in the B-cell proliferation experiments that the BLyS antibody enjoys good biological activity which can inhibit the proliferation of mouse B lymphocytes induced by human BLyS. In vivo detection in mouse demonstrated that the BLyS antibody was able to reduce the increase of B lymphocytes in mouse splenocytes which is caused by BLyS protein. Therefore, the BLyS antibody can be used alone or in combination with other drugs in detecting, diagnosing, treating or screening diseases associated with abnormal expression or dysfunction of BLyS, such as autoimmune diseases, inflammatory diseases, infectious diseases, or proliferative diseases.

### BRIEF DESCRIPTION OF THE DRAWINGS

The features and advantages of the present invention are described below with reference to the drawings.

FIG. 1 shows the detection result of the biological activity of purified hBLyS-ECD. As used herein, RLU represents the relative light unit.

FIG. 2A and FIG. 2B show the detection of expression level of BLyS protein in CHO-K1 recombinant cell line by using flow cytometry analysis method. Among them, 2C4 represents the clone number; 4C4 represents the clone number; antibody refers to the BLyS antibody (purchased from eBioscience); negative control refers to the isotype antibody control.

FIG. 3 shows the serum antibody titer of BALB/c and SJL mice after immunization with immunogen in ELISA detection assay.

FIG. 4 shows the activity of biotinylated hBLyS-ECD in ELISA detection assay

FIG. 5A and FIG. 5B show the reactivity of the purified BLyS antibody with biotinylated hBLyS-ECD in ELISA assay.

FIG. 6A and FIG. 6B show the results of enzyme-linked immunosorbent assay for detecting the reaction of the purified BLyS antibody and the BLyS homologous protein APRIL.

FIG. 7 shows the reactivity of purified BLyS antibodies and human BLyS recombinant cells detected by flow cytometry analysis.

FIG. 8 shows the reactivity of purified BLyS antibodies and cynomolgus BLyS recombinant cells using flow cytometry analysis.

FIG. 9A and FIG. 9B show the binding activity of purified BLyS antibody-blocked BLyS receptor BAFF R with biotinylated hBLyS-ECD.

FIG. 10 shows the inhibitory effect of purified BLyS antibodies on the proliferation of mouse B lymphocytes stimulated with hBLyS-ECD.

FIG. 11 shows the inhibitory effect of purified BLyS antibodies on the proliferation of mouse B lymphocytes stimulated with mouse BLyS-ECD.

FIG. 12 shows the reactivity of BLyS chimeric antibody with biotinylated hBLyS-ECD in ELISA assay.

FIG. 13 shows the binding activity of BLyS chimeric antibody-blocked BLyS receptor BAFF R with biotinylated hBLyS-ECD.

FIG. 14 shows the inhibitory effect of BLyS chimeric antibody on the proliferation of mouse B lymphocytes stimulated with hBLyS-ECD.

FIG. 15A-15C shows the effect of BLyS chimeric antibody and human antibody on the proportion of mouse B lymphocytes in splenocytes after hBLyS-ECD stimulation.

FIG. 16 shows the reactivity of BLyS humanized antibody with the biotinylated hBLyS-ECD in ELISA assay.

FIG. 17 shows the binding activity of BLyS humanized antibody-blocked BLyS receptor BAFF R with biotinylated hBLyS-ECD.

FIG. 18 shows the inhibitory effect of BLyS humanized antibody on the proliferation of mouse B lymphocytes stimulated with hBLyS-ECD.

FIG. 19A and FIG. 19B show the effect of BLyS humanized antibody on the proportion of mouse B lymphocytes in splenocytes and the level of IgA in serum after stimulation with hBLyS-ECD.

### DETAILED DESCRIPTION OF THE PREFERRED EMBODIMENT

The following examples further illustrate the present invention and should not be construed to limit the present invention. Experimental methods without indicating specific conditions in the embodiments below can make reference to routine methods and conditions, or make choice according to product instructions.

The term room “temperature” as used in any of embodiment refers to the temperature of operating chamber in which the experiments is conducted, generally 15-30° C.

### Embodiment 1 Preparation of BLyS Antibody by Hybridoma Technology

(i) Preparation of Immunogen

The nucleotide sequence (as shown in SEQ ID No. 131 of Sequence Listing) encoding the amino acid sequence of Ala134-Leu285 in the extracellular domain of human BLyS protein (BLyS-ECD) was cloned into a His-tagged pCPC vector (purchased from Invitrogen, V044-50) and plasmid was prepared according to established standard molecular biology methods as described in [Sambrook, J., Fritsch, E. F., and Maniatis, T. (1989). Molecular Cloning: A Laboratory Manual, Second Edition (Plainview, N.Y.: Cold Spring Harbor Laboratory Press)]. HEK293 cells (purchased from Invitrogen) were transiently transfected (PEI, Polysciences) and expanded at 37° C. using FreeStyle™ 293 (purchased from Invitrogen). After 7 days, the cell culture liquid was collected and the cell components were removed by centrifugation to obtain a culture supernatant containing the extracellular domain of human BLyS protein. The culture supernatant was loaded on a Ni affinity column (purchased from GE Healthcare) while the changes in UV absorbance (A₂₈₀) were monitored with a UV detector. After loading, the Ni affinity column was equilibrated with phosphate buffer (pH 7.4) containing 5% (v/v) sucrose and 0.01% (v/v) Tween-80 until the UV absorbance returned to baseline, and then gradient elution was carried out with 0-500 mM imidazole. The His-tagged extracellular domain of the human BLyS protein eluted from the Ni affinity column was collected, dialyzed and concentrated in an ultrafiltration tube at 4° C. using phosphate buffer (pH 7.4) containing 5% (v/v) sucrose and 0.01% (v/v) Tween-80. The dialyzed protein was sterile-filtered by 0.22 m and aliquoted and stored at −80° C. to obtain the purified extracellular domain of human BLyS protein as immunogen (i.e., hBLyS-ECD). Before being used, the immunogen is subjected to a series of quality control tests, such as detection of concentration, purity, molecular weight, biological activity of the protein and the like.

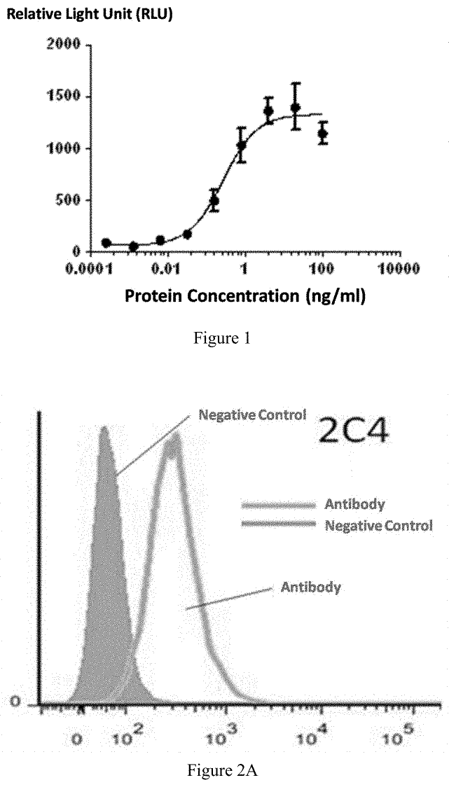

As used herein, the biological activity of the immunogen was tested by B cell proliferation assay (see Embodiment 5 for method). It was shown in FIG. 1 and Table 3 that the immunogen can stimulate the proliferation of mouse B cells.

TABLE 3
Biological Activity Detection of Immunogen

Relative

| light unit | Protein Concentration (ng/mL) | | | | | | | | | |
|---|---|---|---|---|---|---|---|---|---|---|
| (RLU) | 0 | 0.000256 | 0.00128 | 0.00640 | 0.0320 | 0.160 | 0.800 | 4.00 | 20.0 | 100 |
| | 63.64 | 92.57 | 56.70 | 118.02 | 173.56 | 497.54 | 1032.11 | 1365.34 | 1402.37 | 1147.81 |

(ii) Construction of Stable Cell Lines Expressing Human- or Cyno-BLyS

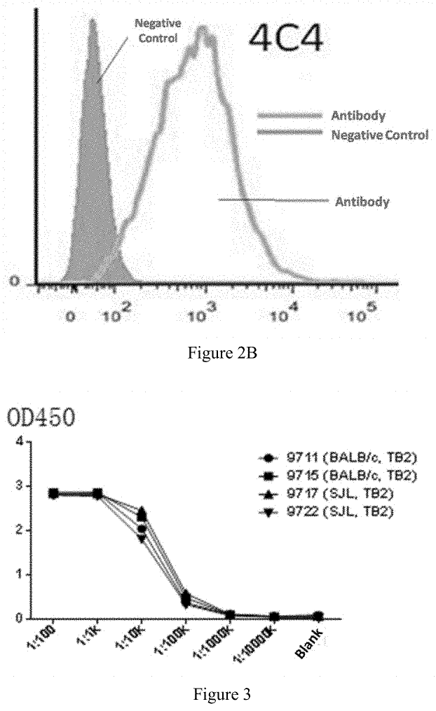

The amino acid sequence of human or cynomolgus BLyS (i.e., hBLyS or cyno BLyS) was mutated from arginine to histidine at position 132 (Genebank accession numbers in NCBI are Q9Y275 and EHH58704.1, respectively), so that the constructed stable cell lines express complete human or cynomolgus BLyS without cleavage. The transfected cells were obtained by cloning the mutated nucleotide sequence (as shown in SEQ ID No. 132 and SEQ ID No. 133) containing full-length amino acid sequence of human or cynomolgus BLyS into the pLVX vector (purchased from Clontech), and then packaging lentivirus and subsequently infecting CHO-K1 cell line (purchased from Invitrogen) by Shanghai GenePharma Co., Ltd. After 72 hours, detection was performed using known BLyS antibody (purchased from eBioscience) by flow cytometry [see Manetta J et al., 2014, J Inflamm Res, 20 (7): 121-131]. When the transfected cells began to express human or cynomolgus BLyS, the cells were subcloned in 96-well plates by limited dilution method and incubated at 37° C. and 5% (v/v) CO₂ for about 2 weeks, and some wells of the monoclonal cells were selected and enlarged into 6-well plates. The amplified clones were further screened by flow cytometry using known BLyS antibody (purchased from eBioscience). Monoclonal cell lines with better growth and higher intensity of fluorescence were selected to expand the culture and cryopreserved in liquid nitrogen, and then human or cynomolgus BLyS stable cell lines were established. The results of specific selection are shown in Table 4 and FIG. 2A-2B, and the positive cell (%) in Table 4 refers to the percentage of positive cells in the total number of cells. It was shown in Table 4 that a series of BLyS-positive CHO-K1 cell lines were constructed.

TABLE 4
FACS screening test results of CHO-K1 cells
expressing human or cynomolgus BLyS protein

| | | BLyS Antibody | | IgG Subtype Control | |
|---|---|---|---|---|---|
| | Clone No. of | Positive | Mean | Positive | Mean |
| | transfected | Cell | fluorescence | Cell | fluorescence |
| No. | cell | (%) | intensity | (%) | intensity |
| 1 | CHO-K1 hBLyS 1B3 | 89.15 | 365 | 0.83 | 31 |
| 2 | CHO-K1 hBLyS 1B4 | 84.87 | 239 | 0.36 | 23 |
| 3 | CHO-K1 hBLyS 2A3 | 77.50 | 198 | 0.18 | 19 |
| 4 | CHO-K1 hBLyS 2C4 | 91.65 | 295 | 0.84 | 39 |
| 5 | CHO-K1 hBLyS 2B3 | 72.46 | 189 | 1.07 | 23 |
| 6 | CHO-K1 hBLyS 3B2 | 87.47 | 295 | 0.24 | 29 |
| 7 | CHO-K1 hBLyS 4B3 | 86.85 | 262 | 0.21 | 27 |
| 8 | CHO-K1 hBLyS 4C2 | 90.78 | 299 | 0.79 | 31 |
| 9 | CHO-K1 cynoBLyS 1A4 | 82.04 | 412 | 0.22 | 31 |
| 10 | CHO-K1 cynoBLyS 1B2 | 87.49 | 412 | 0.10 | 19 |
| 11 | CHO-K1 cynoBLyS 2B3 | 94.66 | 442 | 0.20 | 16 |
| 12 | CHO-K1 cynoBLyS 3B2 | 89.91 | 422 | 0.17 | 19 |
| 13 | CHO-K1 cynoBLyS 4B1 | 92.28 | 742 | 0.71 | 26 |
| 14 | CHO-K1 cynoBLyS 4C2 | 95.00 | 676 | 0.28 | 24 |
| 15 | CHO-K1 cynoBLyS 4C4 | 94.25 | 705 | 0.20 | 27 |

(iii) Preparation of Hybridoma Cells and Screening of Antibody

6-8 weeks old female BALB/c and SJL mice (purchased from Shanghai SLAC Laboratory Animal Co., Ltd.) were feed under SPF conditions. At the time of initial immunization, 0.2 mL of the immunogen (i.e., hBLyS-ECD) collected at step (i) was emulsified in Freund's complete adjuvant and then intraperitoneally injected, that is, each mouse was injected with 100 μg of immunogen. While booster, 0.2 mL of the immunogen was emulsified in incomplete Freund's adjuvant and then injected intraperitoneally, that is, each mouse was injected with 50 μg of immunogen. The interval between the initial immunization and first booster is two weeks and the interval between each booster is three weeks. Blood was collected 1 week after each booster, and the antibody titer and specificity to the immunogen in serum were tested by ELISA. The results were shown in FIG. 3 and Table 5. It was shown in Table 5 that the serum of mice immunized with immunogen bound to the immunogen to a different extent and presented an antigen-antibody reaction, wherein the highest dilution is about 1:1 million. The blank control was 1% (w/w) BSA, wherein the batch refers to the serum of mice on the seventh day after the second booster and the data in the table refers to OD_{450 nm} values.

TABLE 5
Serum antibody titers of BALB/c and SJL mice after
immunized with immunogen detected by ELISA

| | Serum Dilution | | | | | | |
|---|---|---|---|---|---|---|---|
| OD_{450 nm} | | | | | | | Blank |
| Batch | 1:100 | 1:10³ | 1:10⁴ | 1:10⁵ | 1:10⁶ | 1:10⁷ | control |
| 9711 (BALB/c, TB2) | 2.8110 | 2.8547 | 2.0439 | 0.3713 | 0.0994 | 0.0561 | 0.0879 |
| 9715 (BALB/c, TB2) | 2.8624 | 2.8593 | 2.3021 | 0.4848 | 0.0968 | 0.0526 | 0.0488 |
| 9717 (SJL, TB2) | 2.8312 | 2.8201 | 2.4538 | 0.5821 | 0.1084 | 0.0553 | 0.0501 |
| 9722 (SJL, TB2) | 2.7962 | 2.7751 | 1.8081 | 0.3297 | 0.0822 | 0.0516 | 0.0468 |

B. Female BALB/c and SJL mice at 6-8 weeks of age (purchased from Shanghai SLAC Laboratory Animal Co., Ltd.) were feed under SPF conditions. At the time of initial immunization, 25 μL of immunogen (hBLyS-ECD), oligonucleotide (CpG) and GERBU adjuvant were mixed and then injected in tarsal joints of mice, that is, each mouse was injected with 10 μg of immunogen. Upon booster, 25 μL of the immunogen and GERBU adjuvant were mixed and then injected in tarsal joints of mice, that is, each mouse was injected with 5 μg of immunogen. The interval between the initial immunization and first booster is 3-4 days. Blood was collected 1 week after the second booster, and the antibody titer and specificity to the immunogen in serum were measured by ELISA. After the second booster, the serum antibody titer detected by ELISA reached 1:10000 or more.

Before the completion of steps A and B, 50 μg or 5 μg of immunogen were injected into the peritoneal cavity or tarsal joints of each selected last immunized mouse, respectively, and the mice were sacrificed 5 days later and the splenocytes were harvested. NH₄OH was added by a final concentration of 1% (w/w) to lyse the red blood cells in the splenocytes to prepare a splenocyte suspension. The cells were washed three times with centrifugation at 1000 rpm in DMEM basal medium (purchased from Invitrogen), then the viable cells were mixed with mouse myeloma cells SP2/0 (purchased from ATCC) at a ratio of 5:1, and a high-efficiency electrofusion method was used for cell infusion (See METHODS IN ENZYMOLOGY, VOL. 220). The fused cells were diluted into DMEM medium containing 20% (w/w) fetal bovine serum and 1×HAT. Then 1×10⁵ cells in 200 μL DMEM medium was aliquoted into target well of 96-well cell culture plates and incubated in a 5% (v/v) CO₂ incubator at 37° C. After 14 days, the supernatant of the cell fusion plates was screened by indirect ELISA. Positive clones with OD₄₅₀ₙₘ>1.0 in ELISA assay were enlarged to 24-well plates and cultured in HT DMEM containing 10% (w/w) fetal bovine serum at 37° C. under a condition of 5% (v/v) CO₂. After incubating for 3 days, the culture medium enlarged in 24-well plates was centrifuged, the supernatant was collected and subject to the antibody subtype analysis. Among them, the binding activity to hBLyS-ECD was determined by ELISA (see Embodiment 3A and Embodiment 3B respectively for the method of detecting the binding activity), and the blocking activity of the antibody sample on BLyS receptor was determined by ligand-receptor binding assay (see Embodiment 4 for the detection method of blocking activity).

According to the result of 24-well plate screening, hybridoma cells with OD₄₅₀ₙₘ>1.0 in ELISA assay and the rate of BLyS receptor blocking by hybridoma cell culture supernatant reaching 60% in ligand-receptor binding assay were selected as positive clones that meet the requirements herein. Selected hybridoma cells were subcloned in a 96-well plate by limited dilution method and cultured in DMEM medium (purchased from Invitrogen) containing 10% (w/w) FBS at 37° C. under a condition of 5% (v/v) CO₂. 10 days after subcloning, ELISA was used for preliminary screening, and single positive monoclone was selected and expanded into 24-well plate for further culture. After 3 days, the biological activity was evaluated using receptor-ligand binding assay. Where the evaluation criteria is that OD₄₅₀ₙₘ should higher than 1.0 in ELISA assay, and the rate of BLyS receptor block-inhibited by hybridoma cell culture supernatant should reach 60% in ligand-receptor binding assay.

According to the detection results of 24-well plate samples, the optimal clones were selected and expanded in DMEM culture medium (purchased from Invitrogen) containing 10% (w/w) FBS at 37° C. under a condition of 5% (v/v) CO₂. The optimal clones were obtained and cryopreserved in liquid nitrogen, and the optimal hybridoma cells were used for subsequent production and purification of antibody.

(iv) Production and Purification of the Lead Antibody

Since hybridoma cells produce lower concentration of antibody, only about 1-10 g/mL, and the concentration of antibody varies greatly and many biological activity analysis methods were interfered in different level by various proteins produced by cells in culture medium and components of fetal bovine serum in culture medium, so it is necessary to carry out small-scale (1-5 mg) of antibody production and purification.

The hybridoma cells harvested at step (iii) were inoculated into T-75 cell culture flasks and acclimated for 3 generations using a production medium (Hybridoma serum free medium, purchased from Invitrogen). When they were in healthy condition, the cells were inoculated into roller bottle. 500 mL of production medium was poured to each 2-liter roller bottle at a cell density of 1.0×10⁵ cells/mL. The bottle caps were covered and the roller bottles were placed on a rotary machine in a 37° C. incubator at a 3 rpm/min of rotating speed. After 14 days of continuous rotation culture, the cell culture was collected and the cells were removed by filtration through a 0.45 m filter until the culture supernatant were clarified. The clarified culture supernatant can be purified immediately or stored at −30° C.

Monoclonal antibodies in the 300 mL of clarified culture supernatant of hybridoma cells was purified with a 2 mL of Protein G column (purchased from GE Healthcare). The Protein G column was first equilibrated with equilibration buffer (PBS phosphate buffer, pH 7.2) and then the clarified culture supernatant was loaded onto the Protein G column at a flow rate of 3 mL/min. After loading, the Protein G column was washed with equilibration buffer, and the volume of the equilibration buffer was 4 times of the bed volume of Protein G column. The monoclonal antibody bound to Protein G column was eluted with elution buffer (0.1M glycine hydrochloride buffer, pH 2.5) and the elution was monitored by an ultraviolet detector (A280 ultraviolet absorption peak). Eluted monoclonal antibody was collected and the pH was neutralized by adding 10% (v/v) of 1.0 M Tris-HCl buffer. Then elution was dialyzed overnight in PBS phosphate buffer immediately. The next day the buffer was changed once and dialysis was continued for 3 hours. Dialyzed monoclonal antibody was collected and sterile filtered using a 0.22 m filter, and sterile stored the harvested purified BLyS antibody as a lead antibody.

The protein concentration (A280/1.4), purity, endotoxicity (Lonza kit) and the like of purified BLyS antibody was tested and the results were shown in Table 6. It was shown in the result that the lead antibody contained 1.0 EU/mg concentration of endotoxin or less.

TABLE 6
Detection and Analysis of Purified BLyS Antibody

| | Antibody | Protein Conc. | Endotoxin |
|---|---|---|---|
| Clone No. | Purification | (mg/mL) | (EU/mg) |
| 35E6F7C3 | >90% | 0.86 | 0.16 |
| 8E7D9C7F5 | >90% | 1.5 | 0.05 |
| 20D1B6E9E5 | >90% | 0.73 | 0.13 |
| 78C11D2D12 | >90% | 0.956 | 0.26 |
| 89A2G5E7 | >90% | 0.734 | 0.23 |
| 97E7B3F2 | >90% | 0.583 | 0.93 |
| 97A3C2H4 | >90% | 0.504 | 0.30 |
| 67A2E1D10 | >90% | 0.5 | 0.16 |
| 111D10D6G3 | >90% | 0.25 | 0.50 |
| 93C6F10D3 | >90% | 0.485 | 0.66 |

### Embodiment 2 Preparation of BLyS Antibody by Phage Display Technology

(i) Biotinylation of BLyS Protein

Biotin-X-X-NHS (purchased from Sigma Aldrich) and the immunogen prepared in embodiment 1 (hBLyS-ECD) were mixed at a molar ratio of 7:1 and stood for 30 minutes at room temperature before addition of 50 mM final concentration of 1 M NH₄Cl to terminate the reaction. Biotinylated immunogens (i.e., biotinylated hBLyS-ECDs) were then obtained by dialysis overnight in PBS phosphate buffer to remove free biotin. The concentration of biotinylated hBLyS-ECD was determined using BCA protein assay kit (purchased from Pierce).

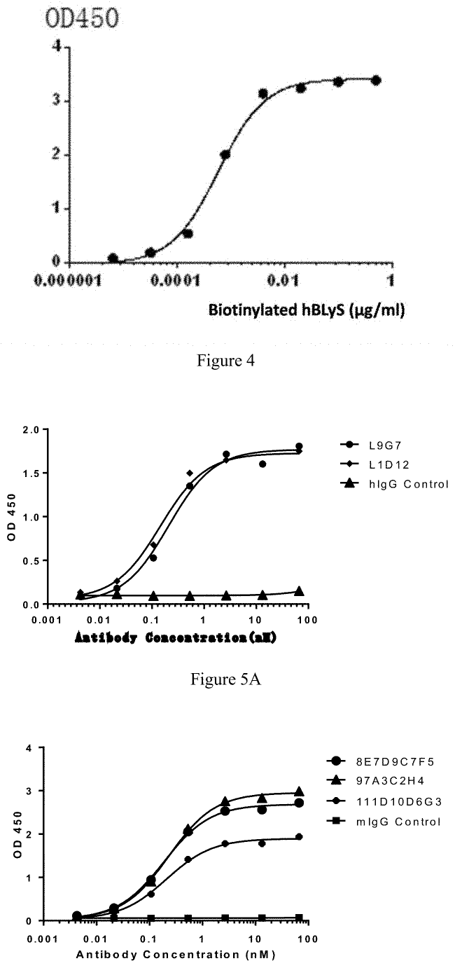

The activity of biotinylated hBLyS-ECD was determined by ELISA. The BLyS antibody (purchased from GSK) was diluted to 1 μg/mL with PBS, 50 μL/well of diluted antibody was aliquoted into the ELISA microplate and incubated overnight at 4° C. After blocking with ELISA blocking solution [PBS phosphate buffer containing 1% (w/v) BSA and 0.05% (v/v) Tween-20, pH 7.4] for 2 hours at 37° C., gradient dilutions of biotinylated hBLyS-ECD were added in and incubated for 1 hour at 37° C. Streptavidin-labeled horseradish peroxidase (purchased from Sigma, product number S5512) was pipetted in and incubated for 30 minutes at room temperature. A 100 μL/well of TMB color developing solution was added in and incubated for 15 minutes at room temperature, then 5 μL of 1N hydrochloric acid was used to stop the color reaction and the value at OD₄₅₀ₙₘ was read by ELISA plate reader. As shown in FIG. 4 and Table 7, the results demonstrated that the biotinylated hBLyS-ECD could bind to BLyS antibodies.

TABLE 7
ELISA detection of the binding of Biotinylated Immunogen to BLyS Antibodies

| | Concentration of Biotinylated Immunogen(ng/mL) | | | | | | | |
|---|---|---|---|---|---|---|---|---|
| OD_{450 nm} | 0.000006 | 0.000032 | 0.00016 | 0.0008 | 0.004 | 0.02 | 0.1 | 0.5 |
| | 0.08 | 0.19 | 0.54 | 2.01 | 3.14 | 3.24 | 3.36 | 3.39 |

(ii) BLyS Antibody Identified by Phage Display Technology

Lead antibody was identified from human naïve single-chain variable fragment (ScFv) phage display library (constructed by Shanghai ChemPartner. Co., Ltd.). Antibody binding to BLyS was obtained by four rounds of biopanning. The detailed process is as follows:

Streptavidin diluted in PBS to a final concentration of 12.5 μg/mL was aliquoted into immuno tubes at 1 mL/tube, and incubated overnight at 4° C. After washing the immuno tubes three times with PBS, the biotinylated hBLyS-ECD prepared at step (i) was aliquoted into a half of the immuno tubes with 50 μg per tube, and shaken at room temperature for 1 hour. After washing three times with PBS buffer, the immuno tubes were blocked for 2 hours at room temperature with 10 mL of 2% (w/v) blocking solution [PBS buffer containing 2% (w/v) skimmed milk powder]. Meanwhile, 1 mL of phage ScFv antibody library and blocking solution were added and shaken for 2 hours at room temperature in the other half of immuno tubes. And the control tube was set by adding the same volume of blocking solution. The blocking solution in control tubes and the immuno tubes adding with biotinylated hBLyS-ECD were removed, and the blocked phage ScFv antibody library was added and the tubes were oscillated at room temperature for 2 hours. Immuno tubes and control tubes were rinsed 5 times with PBST [PBS buffer containing 0.1% (v/v) Tween-20] first and then rinsed 5 times with PBS buffer. Where the time of rinse increased by 5 in each round. After rinsing, 1 mL of 10 μg/mL trypsin was aliquoted into each tube, and the phage bound to the biotinylated hBLyS-ECD was eluted by incubation at 37° C. for 30 minutes. 1 mL of trypsin solution was pipetted into 4 mL of E. coli TG1 (purchased from LUCIGEN) in logarithmic growth phase and allowed for incubating at 37° C. for 30 minutes to obtain a culture solution of TG1. The culture solution of TG1 was serially diluted, plated, and cultured overnight at 37° C. The clones obtained from immuno tubes bound to the biotinylated hBLyS-ECD and control tubes were calculated, and 20-30 clones were selected for sequencing.

Meanwhile, the clones on the plates were washed with 2YT medium (2YT medium was prepared by adding 10 g of yeast extract, 16 g of tryptone and 5 g of NaCl to 1 L of water, and adjusting the pH to 7.0 with NaOH and autoclaving) and collected, then inoculated into fresh medium and cultured until logarithmic phase at 37° C. Helper phage M13K07 (purchased from NEB, Cat. No. N0315S) was added, mixed and stood at 37° C. for 30 minutes. Then, the solution was incubated with shaking at 37° C. for 30 minutes and centrifuged at 4000 rpm for 10 minutes, and the cells were collected and added with fresh medium, then incubated with shaking at 30° C. for 4 hours. After centrifugation at 4000 rpm for 30 minutes, the supernatant was collected, and ¼ volume of 2.5 M NaCl solution containing 5×PEG was added and allowed for placing on ice overnight. After centrifugating at 4000 rpm for 30 minutes at 4° C., the phage precipitation was collected and dissolved in PBS buffer. Residual cell fragments were removed by centrifugating at 10,000 rpm for 10 minutes and the supernatant was collected for biopanning in the next round.

The binding activity of screened scFv antibody with BLyS protein was determined by ELISA. Clones with OD₄₅₀ₙₘ higher than 1.0 were selected for sequencing to get clones with various CDRH3 sequences. Then according to the results of FACS and ligand-receptor binding assay, clones with MFI value higher than 30 in FACS assay and blocking inhibitory rate of cell lysis supernatant on BLyS receptors reaching 60% in ligand-receptor binding assay were selected as the positive clones that meet the criteria.

(iii) Production and Purification of Antibody

Based on the sequencing results of the positive clones (see details in Embodiment 7 below), primers (detailed primer sequences are shown in Table 8) were designed to amplify light chain and heavy chain variable regions by PCR, respectively. A 50 μL of reaction system including 0.5 μL of plasmid extracting from E. coli TG1 transfected with positive clones), 10 pmol of each primer, 0.5 μL of DNA polymerase and a suitable buffer system was set up. The PCR program consisted of pre-denaturation at 95° C. for 2 minutes, 30 cycles of denaturation at 95° C. for 15 seconds, annealing at 55° C. for 30 seconds and extension at 68° C. for 45 seconds, followed by 10 minutes extension at 68° C. to obtain the PCR products. The DNA polymerase used in PCR was purchased from Invitrogen, with Cat. No. 12344, while the buffer system is a buffer system purchased and used with the DNA polymerase as a set. 5 μL of PCR products were used for agarose gel electrophoresis, and positive samples were purified by column recovery kit NucleoSpin® Gel & PCR Clean-up, which is purchased from MACHEREY-NAGEL with Cat. No. 740609. Ligation was carried out as follows: 3 μL of inserted fragment, 0.5 μL of digested expression vector, 0.5 μL of recombinase Exnase and 2 μL of buffer in 10 μL of reaction system reacted at 37° C. for half an hour to obtain the ligated products, namely the constructed recombinant vector. The recombinase was purchased from Vazyme with Cat. No. C112-01/02; the buffer was purchased for using with the recombinase as a set; heavy chain variable region was directionally cloned into the expression vector containing the signal peptide and heavy chain IgG1 constant region of human antibody (the expression vector was purchased from Invitrogen and the recombination process was performed by Shanghai ChemPartner Co., Ltd.), light chain variable region was directionally cloned into the expression vector containing the signal peptide and light chain kappa or lambda (only for the following light chain of the LID12 antibody) constant region of human antibody (the expression vector was purchased from Invitrogen and the recombination was performed by Shanghai ChemPartner Co., Ltd.). 10 μL of the ligation products were aliquoted into 50 μL of competent cells (Ecos 101competent cells, purchased from Yeastern, Cat. No. FYE607) and placed in an ice bath for 30 minutes. After that, above competent cells were heat shocked at 42° C. for 1 minute in water bath and placed on ice for 2 minutes, then mixed with 500 μL of antibiotic-free 2YT medium and resuscitated at 200 RPM for 45 minutes on a shaker at 37° C., and 200 μL of above mix was pipetted to spread on LB solid medium containing 100 μg/mL of ampicillin and overnight incubated in 37° C. incubator. On the next day, colony PCR was performed in a 30 μL of PCR system using primers pTT-EF1a-F and pSV40 (whose nucleotide sequences are shown in SEQ ID No. 134-135, respectively) specific for the expression vector. The reaction system of colony PCR comprises: 1 μL of each primer, 10 μL of PCR pre-mixture (purchased from Novoprotein) and replenish to a total volume of 20 μL with water. Bacterial colony was picked into the PCR system by a pipette tip with continuously pipetting for several times, and then 0.5 μL of which was picked onto another LB solid plate containing 100 μg/mL ampicillin for the preservation of the bacterial strain. After the PCR reaction, 5 μL of products were picked out for detection by agarose gel electrophoresis and the positive samples were sequenced and analyzed [see Kabat, “Sequences of Proteins of Immunological Interest,” National Institutes of Health, Bethesda, Md. (1991)].

TABLE 8
Primer Sequence NO. of Positive Clones

| Clone No. | | Forward Primer | Reverse Primer |
|---|---|---|---|
| 2-1G11 | V_{H} | 176 | 177 |
| | V_{L} | 178 | 179 |
| L9G7 | V_{H} | 180 | 181 |
| | V_{L} | 182 | 183 |
| L1D12 | V_{H} | 184 | 185 |
| | V_{L} | 186 | 187 |

After validating by colony PCR, expression vectors containing the aforesaid recombinant antibody light chain and heavy chain with correct sequences were transiently transfected into FreeStyle™ 293-F cells (purchased from Invitrogen) to produce antibodies. At the time of transfection, the density of 293-F cells should among 1-1.5×10⁶ cells/mL, and 100 μg of cells require 100 μg of aforesaid constructed DNA vectors (the mass ratio of the recombinant light chain vector to the recombinant heavy chain vector is 3:2) and 200 μg of transfection reagent polyethyleneimine (PEI). The DNA vectors and PEI were pipetted into the culture medium respectively, and allowed to stand at room temperature for 5 minutes before filtering through a 0.22-μm of membrane filter, and then the mixture was obtained by mixing DNA vectors and PEI and allowed to stand at room temperature for 15 minutes. Then the mixture was aliquoted into the cells slowly and cultured at 37° C. in 8% (v/v) CO₂ incubator at 120 rpm. The day after transfection, 0.5% (v/v) of peptone was pipetted into the cell culture medium. After 6-7 days, the cell culture medium was centrifuged at 3500 g for 30 minutes. The supernatant was collected and filtered through a 0.22 μm filter.

Monoclonal antibody in 200 mL of clarified supernatant was purified using 1 mL of Protein A column (purchased from GE Healthcare). The Protein A column was first equilibrated with equilibration buffer (PBS phosphate buffer, pH 7.2) and the clarified supernatant was loaded onto the Protein A column at a flow rate of 3 mL/min. After loading, the Protein A column was washed with equilibration buffer, where the volume of the equilibration buffer was 5 times bed volume of Protein A column. BLyS antibody bound to Protein A column was eluted with elution buffer (0.1M glycine hydrochloride buffer, pH 2.5) and the elution was monitored by an ultraviolet detector (A280 ultraviolet absorption peak). Eluted antibody was collected and the pH was neutralized by adding 10% (v/v) of 1.0 M Tris-HCl buffer. Then dialysis was performed immediately in PBS phosphate buffer overnight. The next day the buffer was changed once and dialysis was continued for 3 hours. Dialyzed BLyS antibody was collected and sterile filtered using a 0.22 μm filter, and stored the purified BLyS antibody sterilely as a lead antibody.

The lead antibody was tested for concentration (A280/1.4), purity, and endotoxicity (Lonza kit) of protein, etc, the results were shown in Table 9. It was found that the endotoxin concentration of the lead antibody was 1.0 EU/mg or less.

TABLE 9
Analysis of purified BLyS Antibody

| | | Protein Conc. | Endotoxin |
|---|---|---|---|
| Clone No. | Antibody Conc. | (mg/mL) | (EU/mg) |
| 2-1G11 | >90% | 1.65 | 0.16 |
| L9G7 | >90% | 2.36 | 0.26 |
| L1D12 | >90% | 2.73 | 0.34 |

### Embodiment 3 Detection of the Lead Antibody

A. Detection of the Binding of Antibody to BLyS Protein by Enzyme-Linked Immunosorbent Assay (ELISA)

To determine the binding activity of the lead antibodies harvested from Embodiment 1 and 2 to human BLyS protein and human APRIL protein of the BLyS protein family, respectively, ELISA was performed.

Streptavidin was first diluted to a final concentration of 1.0 μg/mL with PBS, and then 50 μL of diluted streptavidin per well was aliquoted into 96-well ELISA plate, which was subsequently sealed with plastic film and incubated at 4° C. overnight. On the next day the plate was washed four times with the plate washing buffer [PBS buffer containing 0.05% (v/v) Tween20] and added with the blocking solution [PBS buffer containing 0.05% (v/v) Tween20 and 1% (w/w) BSA] and blocked for 2 hours at 37° C. The blocking solution was removed, the biotinylated hBLyS-ECD prepared in Embodiment 2 was diluted with PBS to a final concentration of 50 ng/mL and then aliquoted into the 96-well ELISA plate at 50 μL per well and incubated for 1 hour at 37° C. After rinsing the plate four times with the plate washing buffer [PBS buffer containing 0.05% (v/v) Tween20], 100 μl of the purified lead antibody harvested in Embodiment 1 and 2 was pipetted into each well. After incubation for 1 hour at 37° C., the plate was rinsed four times with the plate washing buffer [PBS buffer containing 0.05% (v/v) Tween20]. HRP-labeled (horseradish peroxidase) secondary antibody (purchased from Sigma) was added in and incubated at 37° C. for 1 hour. The plate was then rinsed four times with the plate washing buffer [PBS buffer containing 0.05% (v/v) Tween20]. 100 μL of TMB substrate was aliquoted into each well and after incubation for 5 minutes at room temperature, 100 μL of stop solution (1.0 N HCl) was aliquoted into each well. A₄₅₀ₙₘ values were read via an ELISA plate reader (SpectraMax M5e purchased from Molecular Devices) and the results were shown in FIGS. 5A-5B and Table 10. Table 10 showed that the purified antibody can bind to the BLyS recombinant protein in ELISA assay. Data shown in the table is OD number at 450 nm.

TABLE 10
ELISA detection of the binding of purified
BLyS antibody with biotinylated hBLyS-ECD

| OD_{450 nm} | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| L9G7 | 1.8062 | 1.6016 | 1.7138 | 1.3508 | 0.5280 | 0.1814 | 0.0853 | 0.0710 |
| L1D12 | 1.7492 | 1.6062 | 1.6476 | 1.4971 | 0.6781 | 0.2618 | 0.1349 | 0.1099 |
| hIgG Control | 0.1483 | 0.1016 | 0.0956 | 0.0916 | 0.0905 | 0.1106 | 0.1082 | 0.1027 |
| 8E7D9C7F5 | 2.7224 | 2.5619 | 2.5304 | 2.0564 | 0.9458 | 0.2909 | 0.1151 | 0.0564 |
| 97A3C2H4 | 2.9812 | 2.8238 | 2.7511 | 2.1167 | 0.8988 | 0.2826 | 0.1053 | 0.0603 |
| 111D10D6G3 | 1.9376 | 1.7802 | 1.7823 | 1.4143 | 0.6127 | 0.1935 | 0.0820 | 0.0617 |
| mIgG Control | 0.0708 | 0.0627 | 0.0659 | 0.0594 | 0.0562 | 0.0595 | 0.0695 | 0.0574 |

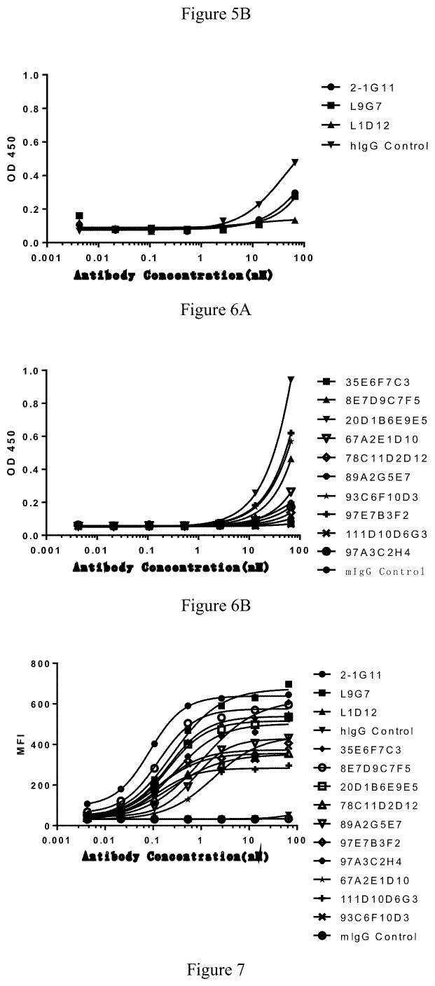

To test whether the BLyS antibody cross-reacted with human APRIL protein, APRIL protein (purchased from R & D Systems) was first diluted to a final concentration of 1.0 μg/mL with PBS, and then 50 μL of which per well was aliquoted into a 96-well ELISA plate, and the plate was subsequently sealed with plastic film and incubated at 4° C. overnight. On the next day the plate was rinsed four times with the plate washing buffer [PBS buffer containing 0.0500 (v/v) Tween20] and a blocking solution [PBS buffer containing 0.05 (v/v) Tween20 and 1 (w/w) BSA] was added, then the protein was blocked at 37° C. for 2 hours. The blocking solution was removed and 100 μL per well of the lead antibodies prepared in Embodiment 1 and 2 were added in. After incubation for 1 hour at 37° C., the plate was rinsed four times with the plate washing buffer [PBS buffer containing 0.05 (v/v) Tween20]. HRP-labeled (horseradish peroxidase) secondary antibody (purchased from Sigma) was added in and incubated at 37° C. for 1 hour. The plate was then rinsed four times with the plate washing buffer [PBS buffer containing 0.050 (v/v) Tween20]. 100 μL of TMB substrate was aliquoted into each well and after incubation at room temperature for 5 minutes, 100 μL of stop solution (1.0 N HCl) was aliquoted into each well. A₄₅₀ₙₘ values were read via an ELISA plate reader (SpectraMax M5e purchased from Molecular Devices) and the results were shown in FIGS. 6A-6B and Table 11. Table 11 showed that the lead antibodies didn't bind to the recombinant APRIL protein which is the most homologous to BLyS protein. As used herein, the IgG control is human IgG and the data in the table is GD number at 450 nm.

TABLE 11-1
ELISA detection of the binding of BLyS antibodies with APRIL proteins

| OD_{450 nm} | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| 2-1G11 | 0.2964 | 0.1374 | 0.09015 | 0.0668 | 0.0699 | 0.07335 | 0.1070 | 0.0847 |
| L9G7 | 0.2755 | 0.1072 | 0.0751 | 0.07405 | 0.0861 | 0.0777 | 0.1606 | 0.0638 |
| L1D12 | 0.1334 | 0.1240 | 0.1019 | 0.0757 | 0.06785 | 0.0854 | 0.1215 | 0.0625 |
| hIgG Control | 0.4768 | 0.2263 | 0.1284 | 0.0793 | 0.0722 | 0.0804 | 0.0693 | 0.0711 |

TABLE 11-2
ELISA detection of the binding of BLyS antibodies with APRIL proteins

| OD_{450 nm} | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| 35E6F7C3 | 0.07025 | 0.0567 | 0.05605 | 0.05435 | 0.05365 | 0.05315 | 0.0553 | 0.05425 |
| 8E7D9C7F5 | 0.46405 | 0.1156 | 0.07025 | 0.0599 | 0.05575 | 0.0565 | 0.05715 | 0.05515 |
| 20D1B6E9E5 | 0.942 | 0.25655 | 0.07755 | 0.0611 | 0.0543 | 0.05465 | 0.0535 | 0.055 |
| 67A2E1D10 | 0.26415 | 0.0898 | 0.0673 | 0.05805 | 0.0588 | 0.05625 | 0.0553 | 0.05305 |
| 78C11D2D12 | 0.13785 | 0.0703 | 0.0613 | 0.05525 | 0.05485 | 0.05345 | 0.0564 | 0.05645 |
| 89A2G5E7 | 0.19595 | 0.0973 | 0.0614 | 0.0565 | 0.0548 | 0.0547 | 0.0578 | 0.06555 |
| 93C6F10D3 | 0.56985 | 0.1712 | 0.07455 | 0.0584 | 0.05505 | 0.0568 | 0.0546 | 0.0568 |
| 97E7B3F2 | 0.6204 | 0.182 | 0.0707 | 0.0556 | 0.0705 | 0.05375 | 0.0535 | 0.0533 |
| 111D10D6G3 | 0.0681 | 0.05725 | 0.05835 | 0.0525 | 0.05355 | 0.05485 | 0.0538 | 0.0547 |
| 97A3C2H4 | 0.17275 | 0.0848 | 0.06165 | 0.05895 | 0.05615 | 0.0559 | 0.0625 | 0.0566 |
| mIgG Control | 0.102 | 0.06245 | 0.05575 | 0.0536 | 0.0533 | 0.05365 | 0.05865 | 0.0716 |

B. Detection of the Binding of Antibody with BLyS-Expressing Cells by Fluorescence-Activated Cell Sorting Assay (FACS)

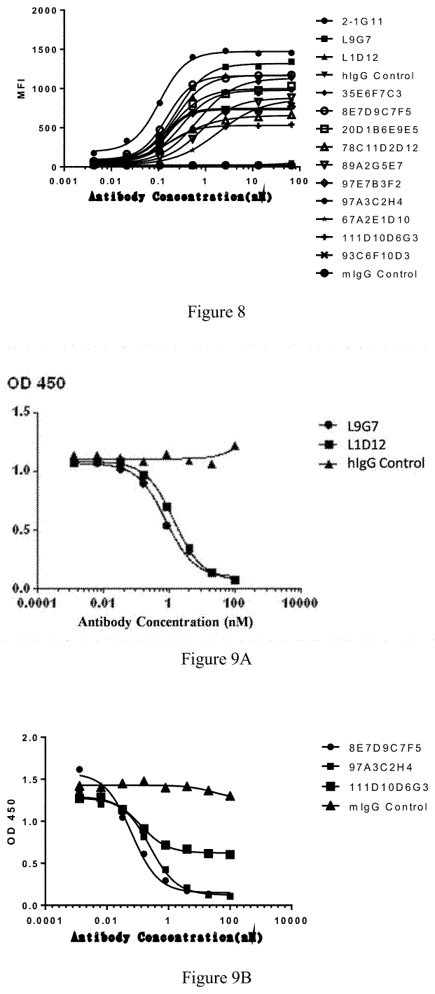

The CHO-K1-hBLyS stable cell line and the CHO-K1-cynoBLyS stable cell line obtained from step (ii) of Embodiment 1 were enlarged and cultured to 90% confluency in T-175 cell culture flask. The medium was aspirated completely, and the cells were rinsed once with PBS buffer (purchased from Invitrogen) and then treated with cell dispersion solution (TrypLE™ Express Enzyme, purchased from Life technology) and collected. The detailed steps are as follows: the cells were rinsed once with PBS buffer, after counting, the cells were diluted to 2×10⁶ cells/ml with PBS buffer, blocked with 2% (w/w) fetal bovine serum and incubated on ice for 30 minutes. 200 μL of which per well was aliquoted into a 96-well FACS plate and centrifuged at 2000 rpm at 4° C., then each 100 μL of the lead antibodies obtained from Embodiment 1 and 2 were aliquoted into each well and incubated on ice for 1 hour. The cells were washed twice with FACS buffer [PBS buffer containing 2% (w/w) FBS], and then mixed with 100 μL per well of fluorescence (Alexa 488) labeled secondary antibody (purchased from Invitrogen) on ice for 1 hour. After centrifuged and rinsed 3 times with FACS buffer, cells were suspended with 200 μL of FACS buffer and the results were detected and analyzed by FACS (FACS Calibur, purchased from BD). The results were shown in FIGS. 7-8 and Tables 12-13. It was shown in Tables 12-13 that the lead antibody could bind to human BLyS protein on the cell surface and the lead antibody could cross-react with cynomolgus BLyS. The data in the table is MFI, the average fluorescence intensity of the cell population.

TABLE 12
Detection of the binding of BLyS antibody to human BLyS
recombinant cells (CHO-K1 hBLyS) by flow cytometry

| MFI | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| 2-1G11 | 645.0 | 632.9 | 627.1 | 590.6 | 402.7 | 180.5 | 106.8 | 185.5 |
| L9G7 | 697.0 | 629.6 | 590.7 | 469.3 | 226.4 | 93.0 | 51.6 | 101.3 |
| L1D12 | 549.9 | 522.4 | 503.3 | 413.1 | 205.1 | 89.5 | 54.4 | 101.2 |
| hIgG Control | 52.0 | 35.1 | 33.1 | 33.4 | 32.9 | 33.6 | 32.3 | 27.2 |
| 35E6F7C3 | 608.6 | 528.3 | 437.4 | 247.3 | 110.4 | 54.9 | 39.2 | 52.4 |
| 8E7D9C7F5 | 596.2 | 569.5 | 532.1 | 504.5 | 282.3 | 122.0 | 67.4 | 151.6 |
| 20D1B6E9E5 | 534.7 | 498.0 | 473.9 | 401.5 | 193.5 | 83.6 | 46.8 | 31.8 |
| 78C11D2D12 | 355.5 | 336.0 | 299.5 | 248.9 | 132.0 | 61.3 | 39.5 | 31.2 |
| 89A2G5E7 | 431.1 | 405.8 | 373.9 | 192.4 | 92.1 | 48.1 | 37.0 | 31.0 |
| 97E7B3F2 | 406.0 | 349.2 | 332.0 | 318.2 | 174.0 | 78.7 | 48.2 | 32.4 |
| 97A3C2H4 | 525.0 | 460.1 | 448.0 | 340.3 | 158.0 | 69.8 | 44.0 | 31.2 |
| 67A2E1D10 | 432.4 | 371.2 | 279.0 | 128.6 | 60.0 | 37.1 | 33.2 | 31.2 |
| 111D10D6G3 | 295.5 | 276.0 | 264.9 | 246.6 | 150.3 | 65.5 | 41.2 | 32.4 |
| 93C6F10D3 | 391.6 | 327.0 | 316.7 | 306.0 | 174.6 | 76.6 | 44.9 | 31.6 |
| mIgG Control | 33.6 | 31.7 | 31.0 | 30.7 | 31.6 | 32.7 | 30.9 | 30.3 |

TABLE 13
Detection of the binding of BLyS antibody to cynomolgus BLyS
recombinant cells (CHO-K1 cynoBLyS) by flow cytometry

| MFI | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| 2-1G11 | 1456.4 | 1449.6 | 1480.8 | 1400.5 | 816.2 | 331.8 | 175.6 | 21.2 |
| L9G7 | 1342.8 | 1282.4 | 1276.1 | 1009.5 | 386.8 | 143.3 | 63.5 | 20.6 |
| L1D12 | 1175.8 | 1133.8 | 1166.9 | 900.2 | 363.7 | 134.7 | 66.7 | 21.2 |
| hIgG Control | 44.2 | 26.7 | 22.7 | 22.6 | 22.8 | 22.4 | 22.0 | 20.7 |
| 35E6F7C3 | 1131.2 | 1080.3 | 953.1 | 501.0 | 176.9 | 66.3 | 32.6 | 20.3 |
| 8E7D9C7F5 | 1173.8 | 1167.6 | 1128.8 | 1051.3 | 476.6 | 164.9 | 74.4 | 19.8 |
| 20D1B6E9E5 | 1034.3 | 978.3 | 933.0 | 808.3 | 349.1 | 118.1 | 50.4 | 19.8 |
| 78C11D2D12 | 664.0 | 635.8 | 594.2 | 480.3 | 234.8 | 106.0 | 37.7 | 20.2 |
| 89A2G5E7 | 884.0 | 828.6 | 725.8 | 355.2 | 124.6 | 53.6 | 32.1 | 21.2 |
| 97E7B3F2 | 770.2 | 709.9 | 704.6 | 687.6 | 288.3 | 112.0 | 53.0 | 22.8 |
| 97A3C2H4 | 987.3 | 943.1 | 955.6 | 681.8 | 242.7 | 120.4 | 44.3 | 20.0 |
| 67A2E1D10 | 842.1 | 738.2 | 517.7 | 213.4 | 76.6 | 35.3 | 24.7 | 20.3 |
| 111D10D6G3 | 539.0 | 524.1 | 510.4 | 477.9 | 221.7 | 108.0 | 38.8 | 20.4 |
| 93C6F10D3 | 784.9 | 714.2 | 729.4 | 705.8 | 308.3 | 112.4 | 46.9 | 21.9 |
| mIgG Control | 21.6 | 20.7 | 21.2 | 20.0 | 19.7 | 19.8 | 20.4 | 19.8 |

C. Determination of Affinity Constant of BLyS Antibody

Affinity constants were determined using a Biacore X100 instrument (purchased from GE Healthcare). Detailed operations and methods referred to the instrument manuals and detailed methods provided by manufacturers, specifically: determining affinity constant using a CM5 chip (Sensor Chip CM5, purchased from GE Healthcare). Firstly, the CM5 chip was activated with 50 mM NaOH, 50 mM NHS and 200 mM EDC mixed by volume ratio of 1:1 sequentially, then anti-human Fc fragment antibody (purchased from Genway) was diluted to 16.4 μg/ml with 10 mM sodium acetate buffer (pH5.0) and coupled reacted with CM5 chip. Then 1 M ethanolamine was injected in chip to block the remaining activation sites. After the antibody to be tested (i.e., the human BLyS antibody prepared by Embodiment 2) was captured by chip, 7 different gradient of immunogen dilutions (i.e., hBLyS-ECD) prepared by Embodiment 1 were injected and the binding and dissociation of antigen with antibody was detected by a Biacore instrument. The dissociation constant and binding constant were then fitted by Biacore X100 Evaluation Software 2.0, while the affinity constant was the ratio of dissociation constant to binding constant. The results were shown in Table 14, indicating that the BLyS antibody has high affinity with hBLyS-ECD.

TABLE 14
Affinity constant of BLyS antibody- hBLyS-ECD

| | Affinity | Binding | Dissociation |
|---|---|---|---|
| | Constant | Constant | Constant |
| Clone No. | K_{D} (nM) | kₐ (1/Ms) | k_{d} (1/s) |
| 2-1G11 | 0.637 | 1.485 × 10⁶ | 9.464 × 10⁻⁴ |
| L9G7 | 0.525 | 4.647 × 10⁵ | 2.439 × 10⁻⁴ |
| L1D12 | 2.420 | 5.610 × 10⁵ | 1.358 × 10⁻³ |

### Embodiment 4 Detection of Purified BLyS Antibody Blocking the Binding of the BLyS Protein to its Receptor BAFF R

BLyS antibody blocking the binding of the BLyS protein to its receptor BAFF R was detected by the receptor-ligand binding assay of BLyS protein.

BAFF R (purchased from R & D Systems) was aliquoted into 96-well ELISA plate at 50 ng per well, and the plate was sealed with plastic film and incubated overnight at 4° C. On the next day the plate was washed four times with the plate washing buffer [PBS buffer containing 0.05% (v/v) Tween20] and added with blocking solution [PBS buffer containing 0.05% (v/v) Tween20 and 1% (w/w) BSA], then blocked for 1 hour at room temperature. The blocking solution was removed and 50 μL per well of the lead antibodies prepared in Embodiment 1 and 2 were added followed by the addition of 50 μL per well of the biotinylated hBLyS-ECD prepared in Embodiment 2. After incubation for 1 hour at 37° C., the plate was washed four times with the plate washing buffer [PBS buffer containing 0.05% (v/v) Tween20]. 100 μL per well of HRP-labeled (horseradish peroxidase) avidin (purchased from Sigma) was added and incubated at 37° C. for 1 hour. The plate was then washed four times with the plate washing buffer [PBS buffer containing 0.05% (v/v) Tween20]. 100 μL of TMB substrate was aliquoted into each well and after incubation for 5 minutes at room temperature, then 100 μL of stop solution (1.0 N HCl) was aliquoted into each well. A₄₅₀ₙₘ values were read with an ELISA plate reader (SpectraMax 384plus, Molecular Device) and the results were shown in FIGS. 9A-9B and Table 15. It was shown in Table 15 that the binding of BLyS protein to its receptor BAFF R could be blocked by the BLyS antibody.

TABLE 15-1
The binding of BLyS protein to its receptor BAFF R blocked by BLyS antibody

| OD_{450 nm} | Antibody Conc. (nM) | | | | | | | |
|---|---|---|---|---|---|---|---|---|
| Clone No. | 100 | 20 | 4 | 0.8 | 0.16 | 0.032 | 0.0064 | 0.00128 |
| L9G7 | 0.07055 | 0.13375 | 0.32685 | 0.54205 | 0.8991 | 1.0243 | 1.0739 | 1.0749 |
| L1D12 | 0.0755 | 0.14205 | 0.35175 | 0.70175 | 0.97195 | 1.08705 | 1.08145 | 1.0776 |
| hIgG Control | 1.22145 | 1.06255 | 1.08975 | 1.1444 | 1.08315 | 1.11255 | 1.1394 | 1.13265 |

TABLE 15-2
The binding of BLyS protein to its receptor BAFF R blocked by BLyS antibody

| OD_{450 nm} | Antibody Conc. (nM) | | | | | | | |
|---|---|---|---|---|---|---|---|---|
| Clone No. | 100 | 20 | 4 | 0.8 | 0.16 | 0.032 | 0.0064 | 0.00128 |
| 8E7D9C7F5 | 0.1098 | 0.1334 | 0.1733 | 0.2944 | 0.6103 | 1.0464 | 1.3822 | 1.6167 |
| 97A3C2H4 | 0.1073 | 0.1246 | 0.2077 | 0.4236 | 0.8155 | 1.1209 | 1.2067 | 1.3356 |
| 111D10D6G3 | 0.6047 | 0.6158 | 0.6699 | 0.7171 | 0.9112 | 1.1410 | 1.2852 | 1.2729 |
| mIgG Control | 1.2984 | 1.3641 | 1.4120 | 1.3967 | 1.4740 | 1.4478 | 1.4018 | 1.4197 |

### Embodiment 5 the Effect of Human or Mouse BLyS Protein Blocking by Purified BLyS Antibody on B Lymphocyte Proliferation Detected by Mouse B Lymphocyte Proliferation Assay

(i) B Lymphocytes Isolated from Mice Spleen

Freshly harvested mouse spleen was ground, resuspended in RPMI 1640 medium (purchased from Invitrogen, Cat. No.: A10491) containing 10% (w/w) fetal bovine serum and filtered with a 70 m cell strainer (purchased from BD), and then centrifuged at 1,500 rpm at 4° C. for 5 minutes. The supernatant was removed and the cell pellets were put on vortex for 15 seconds. Red blood cell lysis buffer (purchased from Sigma) was added in and the mixture was allowed to stand at room temperature for 5 minutes. The mixture was replenished to 15 mL with RPMI 1640 medium containing 10% (w/w) fetal bovine serum and centrifuged at 1500 rpm at 4° C. for 5 minutes to obtain the supernatant. The supernatant was filtered with a 40 m cell strainer (purchased from BD) to obtain a filtered supernatant, and cell counting was followed. Mouse B lymphocytes were isolated using a kit (purchased from Miltenyi Biotec), and the experimental procedures were performed in strict accordance with the instructions of kit. Specific description of the experiment is summarized briefly as follows: The filtered supernatant was centrifuged at 300 g at 4° C. for 10 minutes and the supernatant was removed. 40 μL of buffer [PBS buffer containing 0.5% (w/v) BSA and 2 mM EDTA] and 10 μL of biotinylated antibody mixture that binding with non-B lymphocytes from the kit were aliquoted into each of 10⁷ cells, and incubated at 4° C. for 5 minutes after mixing. 30 μL of buffer [PBS buffer containing 0.5% (w/v) BSA and 2 mM EDTA] and 20 μL of avidin-labeled magnetic beads (purchased from Miltenyi Biotec) were further aliquoted into each of 10′ cells, and mixed and further incubated at 4° C. for 10 minutes to obtain cells suspension. The volume of above mixture was replenished to 500 μL with buffer. LS Separation Columns (purchased from Miltenyi Biotec) were placed on a magnetic stand, rinsed with 3 mL of buffer and then the cell suspension was loaded onto column, and flow-through solution, which is the suspensions containing unlabeled enriched B lymphocytes, was collected. 3 mL of buffer was further loaded onto the column and the flow-through was collected and poured together with the flow-through solution collected by the previous step, then the B lymphocytes isolated from mouse spleen were obtained.

(ii) Proliferation Assay of Mouse B Lymphocytes

50 μl of the B lymphocytes obtained in the step (i) of Embodiment 5 were plated in 96-well cell culture plate at 5×10⁴ cells per well, then 2 μg/ml of F(ab′)2-goat anti-mouse IgM secondary antibody (purchased from Jackson ImmunoResearch) and 10 ng/ml of the immunogen (hBLyS-ECD) prepared in Embodiment 1 or mouse BLyS protein (purchased from R & D Systems), and different concentrations of the lead antibody prepared in Embodiment 1 or 2 were added, and the volume of each reaction well was adjusted to 100 μL. The reaction plate was incubated at 37° C. in a 5% (v/v) CO₂ incubator for 72 hours and the number of viable cells were detected. The number of viable cells was detected by using CellTiter-Glo® Luminescent Cell Viability Assay Kit (purchased from Promega), and the procedure was performed in strict accordance with the kit's instructions. Specific description of the experiment is briefed as follows: the 96-well plate was equilibrated for 30 minutes at room temperature before detection, an equal volume of CellTiter-Glo® reagent was aliquoted into the cell culture medium, and the mixture was placed in a shaker for 2 minutes to induce cell lysis, then the luminescence signal is stabilized by placing the plate at room temperature for 10 minutes, and the values was read via a plate reader (SpectraMax 384plus, Molecular Device).

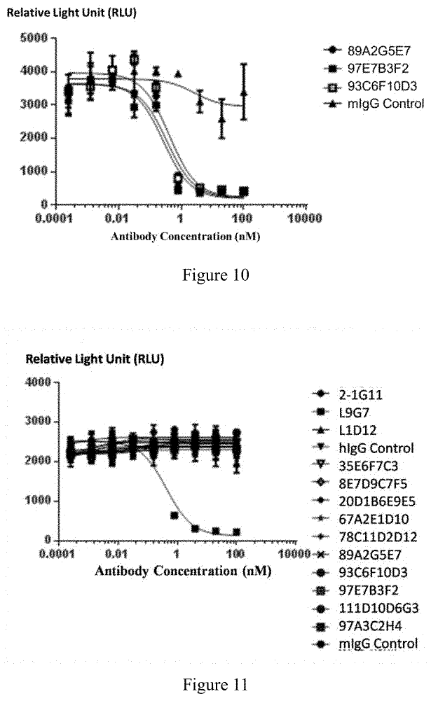

The effect of BLyS antibody on the proliferation of mouse B lymphocyte was detected in the experiment described in step (ii) of Embodiment 5. The results were shown in FIGS. 10-11 and Tables 16-17, and FIG. 10 showed that the antibody to be tested could inhibit mouse B lymphocyte proliferation which was induced by hBLyS-ECD stimulation. It was shown in FIG. 11 that there lacked a cross reaction between mouse BLyS and BLyS antibodies to be tested except antibody L9G7, which could inhibit mouse B lymphocyte proliferation inducing by mouse BLyS.

TABLE 16
Inhibition of BLyS antibody on mouse B lmphocyte proliferation stimulated by hBLyS-ECD

| OD_{450 nm} | Antibody Concentration (nM) | | | | | | | | | |
|---|---|---|---|---|---|---|---|---|---|---|
| Clone No. | 100 | 20 | 4 | 0.8 | 0.16 | 0.032 | 0.0064 | 0.00128 | 0.000256 | 0 |
| 89A2G5E7 | 413.95 | 433.49 | 431.19 | 530.08 | 3267.88 | 3363.31 | 3697.92 | 3704.82 | 3123.00 | 3440.35 |
| 97E7B3F2 | 377.15 | 382.91 | 364.46 | 438.27 | 2992.95 | 2937.59 | 3773.77 | 3758.77 | 3380.47 | 2956.04 |
| 93C6F10D3 | 416.18 | 466.03 | 513.55 | 798.73 | 3528.81 | 4364.64 | 4049.32 | 3546.19 | 3418.68 | 3028.00 |
| mIgG Control | 3395.07 | 2590.61 | 3103.90 | 3945.19 | 4015.39 | 4014.24 | 3696.60 | 3878.43 | 3134.97 | 2733.32 |

TABLE 17
Inhibition of BLyS antibody on mouse B lmphocyte proliferation stimulated by mBLyS

| OD_{450 nm} | Antibody Concentration (nM) | | | | | | | | | |
|---|---|---|---|---|---|---|---|---|---|---|
| Clone No. | 100 | 20 | 4 | 0.8 | 0.16 | 0.032 | 0.0064 | 0.00128 | 0.000256 | 0 |
| 2-1G11 | 2491.49 | 2681.59 | 2461.12 | 2824.44 | 2747.95 | 2473.49 | 2610.72 | 2556.73 | 2453.25 | 2318.27 |
| L9G7 | 220.90 | 243.44 | 307.69 | 640.17 | 2263.13 | 2342.03 | 2461.50 | 2475.02 | 2429.94 | 2104.22 |
| L1D12 | 2454.73 | 2463.75 | 2557.30 | 2409.65 | 2515.59 | 2461.50 | 2504.32 | 2609.14 | 2446.84 | 2343.15 |
| hIgG Control | 2666.96 | 2601.72 | 2584.85 | 2511.74 | 2437.50 | 2501.61 | 2464.49 | 2244.03 | 2224.91 | 2224.91 |
| 35E6F7C3 | 2485.27 | 2563.79 | 2522.83 | 2525.10 | 2406.76 | 2431.79 | 2520.55 | 2339.62 | 2163.23 | 2157.54 |
| 8E7D9C7F5 | 2359.58 | 2347.02 | 2444.10 | 2301.34 | 2352.73 | 2316.18 | 2245.37 | 2244.23 | 2222.53 | 2100.33 |
| 20D1B6E9E5 | 1919.32 | 2239.20 | 2199.22 | 2483.69 | 2116.96 | 2378.58 | 2183.22 | 2174.09 | 2090.69 | 2259.77 |
| 67A2E1D10 | 2067.84 | 2240.35 | 2407.15 | 2489.40 | 2331.74 | 2263.20 | 2111.25 | 2195.79 | 2392.29 | 2121.53 |
| 78C11D2D12 | 2479.25 | 2468.98 | 2338.85 | 2507.78 | 2409.62 | 2389.07 | 2244.11 | 2384.51 | 2205.30 | 1991.85 |
| 89A2G5E7 | 2478.80 | 2636.99 | 2481.07 | 2644.96 | 2368.40 | 2393.44 | 2166.96 | 2272.80 | 2166.96 | 2087.29 |
| 93C6F10D3 | 2742.84 | 2617.65 | 2638.13 | 2527.74 | 2542.53 | 2544.81 | 2509.53 | 2293.29 | 2313.77 | 2311.50 |
| 97E7B3F2 | 2307.96 | 2289.69 | 2413.03 | 2307.96 | 2360.50 | 2575.19 | 2311.39 | 2152.65 | 2197.19 | 2129.81 |
| 111D10D6G3 | 2355.34 | 2538.19 | 2714.19 | 2518.77 | 2290.20 | 2318.77 | 2130.21 | 2151.92 | 2303.92 | 2115.35 |
| 97A3C2H4 | 2322.00 | 2396.38 | 2434.15 | 2414.69 | 2299.11 | 2367.77 | 2251.04 | 2199.55 | 2206.41 | 2162.93 |
| mIgG Control | 2354.26 | 2361.11 | 2437.57 | 2520.88 | 2326.87 | 2320.03 | 2415.89 | 2272.10 | 2112.33 | 2192.21 |

### Embodiment 6 Identification of BLyS Antibody Binding Epitopes

The purified human BLyS antibody and hybridoma antibody were subjected to competitive enzyme-linked immunosorbent assay and the epitopes binding by different antibodies were analyzed.

Purified BLyS antibody was first diluted to a final concentration of 1.0 μg/mL with PBS, then aliquoted into a 96-well ELISA plate at 50 μL per well, and the plate was subsequently sealed with plastic film and incubated at 4° C. overnight. On the next day the plate was rinsed four times with the plate washing buffer [PBS buffer containing 0.05% (v/v) Tween20] and mixed with blocking solution [PBS buffer containing 0.05% (v/v) Tween20 and 1% (w/w) BSA], and then blocked at 37° C. for 1 hour. The blocking solution was removed, and the competitive antibody and IgG control were diluted to a final concentration of 40 μg/mL with PBS and then aliquoted into the 96-well ELISA plate at 50 μL per well. The biotinylated hBLyS-ECD was then diluted with PBS to a final concentration of 2 ng/mL and pipetted into the 96-well ELISA plate at 50 μL per well. The final concentration of competitive antibody and hBLyS-ECD was 20 μg/mL and 1 ng/mL, respectively. After incubation at 37° C. for 1 hour, the plate was rinsed four times with the plate washing buffer [PBS buffer containing 0.05% (v/v) Tween20]. 100 μL per well of HRP-labeled (horseradish peroxidase) streptavidin (purchased from Sigma) was added into the plate and the plate was incubated at 37° C. for 1 hour. The plate was then washed four times with the plate washing buffer [PBS buffer containing 0.05% (v/v) Tween20]. 100 μL of TMB substrate was aliquoted into each well and after incubation at room temperature for 5 minutes, 100 μL of stop solution (1.0 N HCl) was aliquoted into each well. A₄₅₀ₙₘ values were read by an ELISA plate reader (SpectraMax M5e, purchased from Molecular Device) and the results were shown in Tables 18A-18B. It was shown in the tables that the BLyS antibodies were competitive to each other in various degrees, indicating that the binding of antibodies to different epitopes. The data shown in the table is the inhibitory rate (%) of the binding level of the original antibody to hBLyS-ECD after addition of the competitive antibody.

TABLE 18A
BLyS human antibody compete to bind with hBLyS-ECD

| Clone No. | 2-1G11 | L1D12 | L9G7 |
|---|---|---|---|
| 2-1G11 | 82 | 91 | 79 |
| L1D12 | 94 | 94 | 90 |
| L9G7 | 96 | 97 | 95 |

TABLE 18B
Purified BLyS antibody compete to bind with hBLyS-ECD

| Clone No. | 8E7D9C7F5 | 93C6F10D3 | 97A3C2H4 | 67A2E1D10 | 78C11D2D12 | 111D10D6G3 | 35E6F7C3 |
|---|---|---|---|---|---|---|---|
| 8E7D9C7F5 | 92 | 85 | 89 | 96 | 94 | 96 | 40 |
| 93C6F10D3 | 94 | 79 | 90 | 97 | 92 | 95 | 42 |
| 97A3C2H4 | 93 | 93 | 89 | 96 | 96 | 93 | 55 |
| 67A2E1D10 | 63 | 44 | 75 | 93 | 80 | 86 | 18 |
| 78C11D2D12 | 37 | 58 | 46 | 95 | 88 | 95 | 29 |
| 111D10D6G3 | 27 | 41 | 26 | 96 | 86 | 95 | 22 |
| 35E6F7C3 | 27 | 33 | 26 | 33 | 36 | 38 | 91 |

### Embodiment 7 Sequencing of Light Chain and Heavy Chain Variable Region and Preparation of Mouse-Human Chimeric Antibody

Total RNA isolation: after the culture supernatant of subclones corresponding to the lead antibodies selected by Embodiment 1 were tested for antigen binding (i.e., after completing the detection and activity determination of Embodiments 3-5), 5×10⁷ hybridoma cells were collected by centrifugation, mixed with 1 mL of Trizol and transferred to 1.5 mL centrifuge tube, and allowed to stand at room temperature for 5 minutes; subsequently 0.2 mL of chloroform was added and oscillated for 15 seconds, the tubes were centrifuged at 12000 g and 4° C. for 5 minutes after standing for 2 minutes, and supernatant was transferred to a new 1.5 mL of centrifuge tube; 0.5 mL of isopropanol was added, the liquid in the tube was mixed gently and centrifuged at 12000 g and 4° C. for 15 minutes after standing at room temperature for 10 minutes, and the supernatant was removed; then 1 mL of 75% (v/v) ethanol was added in, the pellets were rinsed gently and centrifuged at 12000 g and 4° C. for 5 minutes, the supernatant was removed, the pellets were air dried and dissolved in DEPC-treated H₂O (placed in a 55° C. of water bath for 10 minutes to promote dissolution), then the total RNA was obtained.

Reverse transcription and PCR: 1 μg of total RNA was took and the reverse transcriptase was added, a 20 μL system was set up and reacted at 42° C. for 60 minutes, the reaction was terminated at 85° C. for 10 minutes. A 50 μL PCR system was set with 1 μL of cDNA, 25 pmol of each primer, 1 μL of DNA polymerase, 250 mol of dNTPs and a compatible buffer system. PCR program consisted of pre-denaturing at 95° C. for 3 minutes, 35 cycles of denaturing at 95° C. for 30 seconds, annealing at 55° C. for 30 seconds and extending at 72° C. for 35 seconds, followed by extension at 72° C. for 5 minutes to obtain a PCR product. The kit used for reverse transcription was PrimeScript RT Master Mix purchased from Takara, with Cat. No. RR036; the kit used for PCR including the Q5 high-fidelity enzyme was purchased from NEB, with Cat. No. M0492.

Cloning and sequencing: 5 μL of PCR product was used to test by agarose gel electrophoresis, and positive samples were purified by column recovery kit NucleoSpin® Gel & PCR Clean-up, purchased from MACHEREY-NAGEL with a Cat. No. of 740609. Ligation was carried out by using 50 ng of sample, 50 ng of T vector, 0.5 μL of ligase, 1 μL of buffer being brought to a final reaction system volume of 10 μL, and reacted at 16° C. for half an hour to obtain the ligated product, wherein the ligation kit is T4 DNA ligase purchased from NEB with a Cat. No. of M0402; 5 μL of the ligation product was pipetted into 100 μL of competent cells (Ecos 101competent cells, purchased from Yeastern, Cat. No. FYE607) and ice-cooled for 5 minutes. Subsequently the competent cells were heat shocked at 42° C. for 1 minute in water bath and placed on ice for 1 minutes, then 650 μL of antibiotic-free SOC medium was added and the competent cells were resuscitated at 200 RPM on a shaker at 37° C. for 30 minutes, 200 μL of cells suspension was pipetted and spread on LB solid medium containing antibiotic and cultured overnight in 37° C. incubator. On the next day, colony PCR was performed in a 30 μL PCR system using primers M13F and M13R specifically designed for T vector, bacterial colonies were picked by a tip and pipetted into the PCR system and mixed, and 0.5 μL of suspension was picked onto another LB solid plate containing 100 μg/mL ampicillin to preserve the bacterial strain; 5 μL of product was subjected to the detection of agarose gel electrophoresis when the PCR reaction is completed, and the positive samples were sequenced and analyzed [see Kabat, “Sequences of Proteins of Immunological Interest,” National Institutes of Health, Bethesda, Md. (1991)]. The sequencing results were shown in Tables 19-20.

Cloning and sequencing of the human BLyS antibodies prepared by phage display of the present invention could be found in section (iii) of Embodiment 2, and these sequencing results were also included in Tables 19-20.

TABLE 19
BLyS Antibody Amino Acid SEQ ID NO.

| | Heavy Chain Protein | | | | Light Chain Protein | | | |
|---|---|---|---|---|---|---|---|---|
| | Variable | | | | Variable | | | |
| Clone No. | Region | CDR1 | CDR2 | CDR3 | Region | CDR1 | CDR2 | CDR3 |
| 2-1G11 | 1 | 2 | 3 | 4 | 5 | 6 | 7 | 8 |
| L9G7 | 9 | 10 | 11 | 12 | 13 | 14 | 15 | 16 |
| L1D12 | 17 | 18 | 19 | 20 | 21 | 22 | 23 | 24 |
| 35E6F7C3 | 25 | 26 | 27 | 28 | 29 | 30 | 31 | 32 |
| 8E7D9C7F5 | 33 | 34 | 35 | 36 | 37 | 38 | 39 | 40 |
| 20D1B6E9E5 | 41 | 42 | 43 | 44 | 45 | 46 | 47 | 48 |
| 78C11D2D12 | 49 | 50 | 51 | 52 | 53 | 54 | 55 | 56 |
| 89A2G5E7 | 57 | 58 | 59 | 60 | 61 | 62 | 63 | 64 |
| 97E7B3F2 | 65 | 66 | 67 | 68 | 69 | 70 | 71 | 72 |
| 97A3C2H4 | 73 | 74 | 75 | 76 | 77 | 78 | 79 | 80 |
| 67A2E1D10 | 81 | 82 | 83 | 84 | 85 | 86 | 87 | 88 |
| 111D10D6G3 | 89 | 90 | 91 | 92 | 93 | 94 | 95 | 96 |
| 93C6F10D3 | 97 | 98 | 99 | 100 | 101 | 102 | 103 | 104 |

As used herein, the numbers in Table 19 are the sequence numbers in the sequence listing, for example, the amino acid sequence of heavy chain variable region of 2-1G11 is shown in SEQ ID No. 1, and the amino acid sequence of heavy chain CDR1 of 2-1G11 is shown in SEQ ID No. 2.

TABLE 20
Nucleotide SEQ ID NO. of BLyS Antibody Gene

| | Heavy Chain | Light Chain |
|---|---|---|
| Clone No. | variable region | variable region |
| 2-1G11 | 105 | 106 |
| L9G7 | 107 | 108 |
| L1D12 | 109 | 110 |
| 35E6F7C3 | 111 | 112 |
| 8E7D9C7F5 | 113 | 114 |
| 20D1B6E9E5 | 115 | 116 |
| 78C11D2D12 | 117 | 118 |
| 89A2G5E7 | 119 | 120 |
| 97E7B3F2 | 121 | 122 |
| 97A3C2H4 | 123 | 124 |
| 67A2E1D10 | 125 | 126 |
| 111D10D6G3 | 127 | 128 |
| 93C6F10D3 | 129 | 130 |

As used herein, the numbers in Table 20 are the sequence numbers in the sequence listing, for example, the nucleotide sequence of the heavy chain variable region of 2-1G11 is shown in SEQ ID No.105, and the nucleotide sequence of the light chain variable region of 2-1G11 is shown in SEQ ID No.106.

The nucleotide sequence encoding the heavy chain CDR1 of 2-1G11 is the sequence from 76th to 105th base shown in SEQ ID No.105 of the Sequence Listing.

The nucleotide sequence encoding the heavy chain CDR2 of 2-1G11 is the sequence from 148th to 198th base shown in SEQ ID No.105 of the Sequence Listing.

The nucleotide sequence encoding the heavy chain CDR3 of 2-1G11 is the sequence from 295th to 342nd base shown in SEQ ID No.105 of the Sequence Listing.

The nucleotide sequence encoding the light chain CDR1 of 2-1G11 is the sequence from 70th to 102nd base shown in SEQ ID No.106 of the Sequence Listing.

The nucleotide sequence encoding the light chain CDR2 of 2-1G11 is the sequence from 148th to 168th base shown in SEQ ID No.106 of the Sequence Listing.

The nucleotide sequence encoding the light chain CDR3 of 2-1G11 is the sequence from 265th to 291th base shown in SEQ ID No.106 of the Sequence Listing.

The nucleotide sequence encoding the heavy chain CDR1 of L9G7 is the sequence from 76th to 105th base shown in SEQ ID No.107 of the Sequence Listing.

The nucleotide sequence encoding the heavy chain CDR2 of L9G7 is the sequence from 148th to 198th base shown in SEQ ID No.107 of the Sequence Listing.

The nucleotide sequence encoding the heavy chain CDR3 of L9G7 is the sequence from 295th to 342nd base shown in SEQ ID No.107 of the Sequence Listing.

The nucleotide sequence encoding the light chain CDR1 of L9G7 is the sequence from 70th to 102nd base shown in SEQ ID No.108 of the Sequence Listing.

The nucleotide sequence encoding the light chain CDR2 of L9G7 is the sequence from 148th to 168th base shown in SEQ ID No.108 of the Sequence Listing.

The nucleotide sequence encoding the light chain CDR3 of L9G7 is the sequence from 265th to 291nd base shown in SEQ ID No.108 of the Sequence Listing.

The nucleotide sequence encoding the heavy chain CDR1 of L1D12 is the sequence from 76th to 105th base shown in SEQ ID No.109 of the Sequence Listing.

The nucleotide sequence encoding the heavy chain CDR2 of L1D12 is the sequence from 148th to 195th base shown in SEQ ID No.109 of the Sequence Listing.

The nucleotide sequence encoding the heavy chain CDR3 of L1D12 is the sequence from 292nd to 330nd base shown in SEQ ID No.109 of the Sequence Listing.

The nucleotide sequence encoding the light chain CDR1 of L1D12 is the sequence from 67th to 99th base shown in SEQ ID No.110 of the Sequence Listing.

The nucleotide sequence encoding the light chain CDR2 of L1D12 is the sequence from 145th to 165th base shown in SEQ ID No.110 of the Sequence Listing.

The nucleotide sequence encoding the light chain CDR3 of L1D12 is the sequence from 262nd to 297th base shown in SEQ ID No.110 of the Sequence Listing.

The nucleotide sequence encoding the heavy chain CDR1 of 35E6F7C3 is the sequence from 76th to 105th base shown in SEQ ID No.111 of the Sequence Listing.

The nucleotide sequence encoding the heavy chain CDR2 of 35E6F7C3 is the sequence from 148th to 198th base shown in SEQ ID No.111.

The nucleotide sequence encoding the heavy chain CDR3 of 35E6F7C3 is the sequence from 295nd to 321th base shown in SEQ ID No.111 of the Sequence Listing.

The nucleotide sequence encoding the light chain CDR1 of 35E6F7C3 is the sequence from 70th to 99th base shown in SEQ ID No.112 of the Sequence Listing.

The nucleotide sequence encoding the light chain CDR2 of 35E6F7C3 is the sequence from 145th to 165th base shown in SEQ ID No.112 of the Sequence Listing.

The nucleotide sequence encoding the light chain CDR3 of 35E6F7C3 is the sequence from 262nd to 288th base shown in SEQ ID No.112 of the Sequence Listing.

The nucleotide sequence encoding the heavy chain CDR1 of 8E7D9C7F5 is the sequence from 76th to 105th base shown in SEQ ID No.113 of the Sequence Listing.

The nucleotide sequence encoding the heavy chain CDR2 of 8E7D9C7F5 is the sequence from 148th to 198th base shown in SEQ ID No.113 of the Sequence Listing.

The nucleotide sequence encoding the heavy chain CDR3 of 8E7D9C7F5 is the sequence from 295nd to 342nd base shown in SEQ ID No.113 of the Sequence Listing.

The nucleotide sequence encoding the light chain CDR1 of 8E7D9C7F5 is the sequence from 70th to 114th base shown in SEQ ID No.114 of the Sequence Listing.

The nucleotide sequence encoding the light chain CDR2 of 8E7D9C7F5 is the sequence from 160th to 180th base shown in SEQ ID No.114 of the Sequence Listing.

The nucleotide sequence encoding the light chain CDR3 of 8E7D9C7F5 is the sequence from 277nd to 303th base shown in SEQ ID No.114 of the Sequence Listing.

The nucleotide sequence encoding the heavy chain CDR1 of 20D1B6E9E5 is the sequence from 76th to 105th base shown in SEQ ID No.115 of the Sequence Listing.

The nucleotide sequence encoding the heavy chain CDR2 of 20D1B6E9E5 is the sequence from 148th to 198th base shown in SEQ ID No.115 of the Sequence Listing.

The nucleotide sequence encoding the heavy chain CDR3 of 20D1B6E9E5 is the sequence from 295nd to 339th base shown in SEQ ID No.115 of the Sequence Listing.

The nucleotide sequence encoding the light chain CDR1 of 20D1B6E9E5 is the sequence from 70th to 117th base shown in SEQ ID No.116 of the Sequence Listing.

The nucleotide sequence encoding the light chain CDR2 of 20D1B6E9E5 is the sequence from 163th to 183th base shown in SEQ ID No.116 of the Sequence Listing.

The nucleotide sequence encoding the light chain CDR3 of 20D1B6E9E5 is the sequence from 280nd to 306th base shown in SEQ ID No.116 of the Sequence Listing.

The nucleotide sequence encoding the heavy chain CDR1 of 78C11D2D12 is the sequence from 76th to 105th base shown in SEQ ID No.117 of the Sequence Listing.

The nucleotide sequence encoding the heavy chain CDR2 of 78C11D2D12 is the sequence from 148th to 198th base shown in SEQ ID No.117 of the Sequence Listing.

The nucleotide sequence encoding the heavy chain CDR3 of 78C11D2D12 is the sequence from 295nd to 315th base shown in SEQ ID No.117 of the Sequence Listing.

The nucleotide sequence encoding the light chain CDR1 of 78C11D2D12 is the sequence from 70th to 99th base shown in SEQ ID No.118 of the Sequence Listing.

The nucleotide sequence encoding the light chain CDR2 of 78C11D2D12 is the sequence from 145th to 165th base shown in SEQ ID No.118 of the Sequence Listing.

The nucleotide sequence encoding the light chain CDR3 of 78C11D2D12 is the sequence from 262nd to 288th base shown in SEQ ID No.118 of the Sequence Listing.

The nucleotide sequence encoding the heavy chain CDR1 of 89A2G5E7 is the sequence from 76th to 105th base shown in SEQ ID No.119 of the Sequence Listing.

The nucleotide sequence encoding the heavy chain CDR2 of 89A2G5E7 is the sequence from 148th to 195th base shown in SEQ ID No.119 of the Sequence Listing.

The nucleotide sequence encoding the heavy chain CDR3 of 89A2G5E7 is the sequence from 292nd to 327th base shown in SEQ ID No.119 of the Sequence Listing.

The nucleotide sequence encoding the light chain CDR1 of 89A2G5E7 is the sequence from 70th to 105th base shown in SEQ ID No.120 of the Sequence Listing.

The nucleotide sequence encoding the light chain CDR2 of 89A2G5E7 is the sequence from 151th to 171th base shown in SEQ ID No.120 of the Sequence Listing.

The nucleotide sequence encoding the light chain CDR3 of 89A2G5E7 is the sequence from 268nd to 294th base shown in SEQ ID No.120 of the Sequence Listing.

The nucleotide sequence encoding the heavy chain CDR1 of 97E7B3F2 is the sequence from 76th to 105th base shown in SEQ ID No.121 of the Sequence Listing.

The nucleotide sequence encoding the heavy chain CDR2 of 97E7B3F2 is the sequence from 148th to 198th base shown in SEQ ID No.121 of the Sequence Listing.

The nucleotide sequence encoding the heavy chain CDR3 of 97E7B3F2 is the sequence from 295nd to 321th base shown in SEQ ID No.121 of the Sequence Listing.

The nucleotide sequence encoding the light chain CDR1 of 97E7B3F2 is the sequence from 70th to 117th base shown in SEQ ID No.122 of the Sequence Listing.

The nucleotide sequence encoding the light chain CDR2 of 97E7B3F2 is the sequence from 163th to 183th base shown in SEQ ID No.122 of the Sequence Listing.

The nucleotide sequence encoding the light chain CDR3 of 97E7B3F2 is the sequence from 280nd to 306th base shown in SEQ ID No.122 of the Sequence Listing.

The nucleotide sequence encoding the heavy chain CDR1 of 97A3C2H4 is the sequence from 76th to 105th base shown in SEQ ID No.123 of the Sequence Listing.

The nucleotide sequence encoding the heavy chain CDR2 of 97A3C2H4 is the sequence from 148th to 198th base shown in SEQ ID No.123 of the Sequence Listing.

The nucleotide sequence encoding the heavy chain CDR3 of 97A3C2H4 is the sequence from 295nd to 327th base shown in SEQ ID No.123 of the Sequence Listing.

The nucleotide sequence encoding the light chain CDR1 of 97A3C2H4 is the sequence from 70th to 102th base shown in SEQ ID No.124 of the Sequence Listing.

The nucleotide sequence encoding the light chain CDR2 of 97A3C2H4 is the sequence from 148th to 168th base shown in SEQ ID No.124 of the Sequence Listing.

The nucleotide sequence encoding the light chain CDR3 of 97A3C2H4 is the sequence from 265nd to 291th base shown in SEQ ID No.124 of the Sequence Listing.

The nucleotide sequence encoding the heavy chain CDR1 of 67A2E1D10 is the sequence from 76th to 105th base shown in SEQ ID No.125 of the Sequence Listing.

The nucleotide sequence encoding the heavy chain CDR2 of 67A2E1D10 is the sequence from 148th to 198th base shown in SEQ ID No.125 of the Sequence Listing.

The nucleotide sequence encoding the heavy chain CDR3 of 67A2E1D10 is the sequence from 295nd to 333th base shown in SEQ ID No.125 of the Sequence Listing.

The nucleotide sequence encoding the light chain CDR1 of 67A2E1D10 is the sequence from 70th to 102nd base shown in SEQ ID No.126 of the Sequence Listing.

The nucleotide sequence encoding the light chain CDR2 of 67A2E1D10 is the sequence from 148th to 168th base shown in SEQ ID No.126 of the Sequence Listing.

The nucleotide sequence encoding the light chain CDR3 of 67A2E1D10 is the sequence from 265nd to 291th base shown in SEQ ID No.126 of the Sequence Listing.

The nucleotide sequence encoding the heavy chain CDR1 of 111D10D6G3 is the sequence from 76th to 105th base shown in SEQ ID No.127 of the Sequence Listing.

The nucleotide sequence encoding the heavy chain CDR2 of 111D10D6G3 is the sequence from 148th to 198th base shown in SEQ ID No.127 of the Sequence Listing.

The nucleotide sequence encoding the heavy chain CDR3 of 111D10D6G3 is the sequence from 295nd to 309th base shown in SEQ ID No.127 of the Sequence Listing.

The nucleotide sequence encoding the light chain CDR1 of 111D10D6G3 is the sequence from 70th to 102th base shown in SEQ ID No.128 of the Sequence Listing.

The nucleotide sequence encoding the light chain CDR2 of 111D10D6G3 is the sequence from 148th to 168th base shown in SEQ ID No.128 of the Sequence Listing.

The nucleotide sequence encoding the light chain CDR3 of 111D10D6G3 is the sequence from 265nd to 291th base shown in SEQ ID No.128 of the Sequence Listing.

The nucleotide sequence encoding the heavy chain CDR1 of 93C6F10D3 is the sequence from 76th to 105th base shown in SEQ ID No.129 of the Sequence Listing.

The nucleotide sequence encoding the heavy chain CDR2 of 93C6F10D3 is the sequence from 148th to 198th base shown in SEQ ID No.129 of the Sequence Listing.

The nucleotide sequence encoding the heavy chain CDR3 of 93C6F10D3 is the sequence from 295nd to 336th base shown in SEQ ID No.129 of the Sequence Listing.

The nucleotide sequence encoding the light chain CDR1 of 93C6F10D3 is the sequence from 70th to 102th base shown in SEQ ID No.130 of the Sequence Listing.

The nucleotide sequence encoding the light chain CDR2 of 93C6F10D3 is the sequence from 148th to 168th base shown in SEQ ID No.130 of the Sequence Listing.

The nucleotide sequence encoding the light chain CDR3 of 93C6F10D3 is the sequence from 265nd to 291th base shown in SEQ ID No.130 of the Sequence Listing.

Preparation of mouse-human chimeric BLyS antibody: According to the sequencing results of above steps, the antibody heavy chain variable region and light chain variable region sequences were obtained. Production and preparation of mouse-human chimeric BLyS antibody referred to step (iii) of Embodiment 2, including: 1. the preparation of the recombinant vector: the heavy chain variable region was directionally cloned into the expression vector containing the signal peptide and the heavy chain IgG1 constant region of human antibody (wherein the expression vector was purchased from Invitrogen and the recombination process was performed by Shanghai ChemPartner Co., Ltd.), the light chain variable region was directionally cloned into the expression vector containing the signal peptide and the light chain kappa constant region of human antibody (wherein the expression vector was purchased from Invitrogen and the recombination process was performed by Shanghai ChemPartner Co., Ltd.); 2, cell transfection; 3, antibody purification. The harvested BLyS chimeric antibody was characterized (methods referred to Embodiments 3-5). Each harvested chimeric antibody was named with the initial “c” before the clone number of corresponding lead antibody, for example, the chimeric antibody c8E7D9C7F5 corresponds to the lead antibody 8E7D9C7F5.

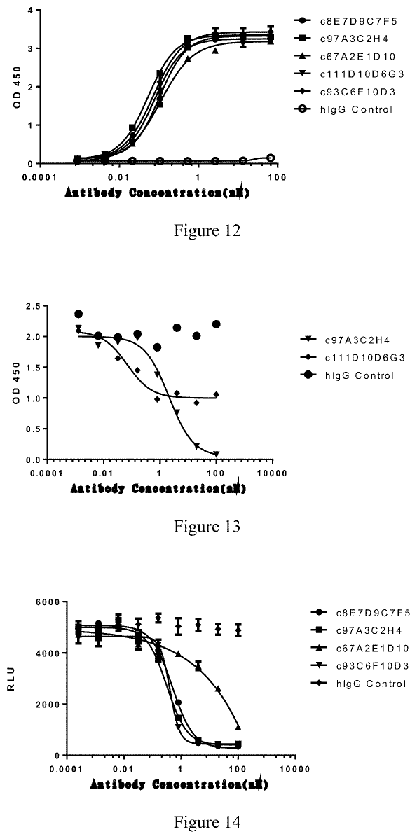

The results were shown in FIG. 12 and Table 21, and it was shown in Table 21 that the chimeric antibody could bind to BLyS recombinant protein in ELISA assay. Data shown in the table is the OD value at 450 nm.

TABLE 21
ELISA detection of the binding of BLyS chimeric
antibody to biotinylated hBLyS-ECD

| OD_{450 nm} | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| c8E7D9C7F5 | 3.3060 | 3.3214 | 3.2359 | 3.0718 | 1.6993 | 0.5743 | 0.1761 | 0.0918 |
| c97A3C2H4 | 3.3024 | 3.3311 | 3.3218 | 3.2608 | 2.3489 | 0.9358 | 0.2502 | 0.1218 |
| c67A2E1D10 | 3.1888 | 3.2390 | 2.9446 | 2.7309 | 1.5835 | 0.5226 | 0.1428 | 0.0848 |
| c111D10D6G3 | 3.4282 | 3.3866 | 3.4048 | 3.1580 | 2.1245 | 0.7471 | 0.2222 | 0.1048 |
| hIgG Control | 0.1436 | 0.0720 | 0.0652 | 0.0620 | 0.0662 | 0.0699 | 0.0798 | 0.0731 |

The binding and dissociation between antibody and antigen was detected via Biacore. The results were shown in Table 22, indicating that the BLyS chimeric antibody has high affinity for hBLyS-ECD.

TABLE 22
Affinity constant of BLyS chimeric antibody-hBLyS-ECD

| | Affinity | Binding | Dissociation |
|---|---|---|---|
| | Constant | Constant | Constant |
| Clone No. | K_{D} (nM) | kₐ (1/Ms) | k_{d} (1/s) |
| c35E6F7C3 | 1.454 | 1.905 × 10⁵ | 2.770 × 10⁻⁴ |
| c8E7D9C7F5 | 2.383 | 4.565 × 10⁵ | 1.088 × 10⁻³ |
| c20D1B6E9E5 | 0.642 | 3.294 × 10⁵ | 2.113 × 10⁻⁴ |
| c89A2G5E7 | 0.0857 | 2.128 × 10⁵ | 1.823 × 10⁻⁵ |
| c97E7B3F2 | 0.147 | 3.335 × 10⁵ | 4.892 × 10⁻⁵ |
| c97A3C2H4 | 0.446 | 1.647 × 10⁵ | 7.340 × 10⁻⁵ |
| c67A2E1D10 | 4.990 | 3.195 × 10⁴ | 1.594 × 10⁻⁴ |
| c111D10D6G3 | 0.297 | 1.004 × 10⁵ | 2.984 × 10⁻⁵ |
| c93C6F10D3 | 0.386 | 5.489 × 10⁵ | 2.118 × 10⁻⁴ |

The results were shown in FIG. 13 and Table 23, and it was shown in Table 23 that the BLyS chimeric antibody could block the binding of BLyS protein to its receptor BAFF R.

TABLE 23
The binding of BLyS protein to its receptor
BAFF R was blocked by BLyS chimeric antibody

| OD_{450 nm} | Antibody Conc. (nM) | | | | | | | |
|---|---|---|---|---|---|---|---|---|
| Clone No. | 100 | 20 | 4 | 0.8 | 0.16 | 0.032 | 0.0064 | 0 |
| c97A3C2H4 | 0.0799 | 0.2137 | 0.7615 | 1.3773 | 1.9786 | 1.9078 | 1.8547 | 2.1380 |
| c111D10D6G3 | 1.0556 | 0.9201 | 1.0798 | 0.9776 | 1.4512 | 1.6438 | 2.0103 | 2.0924 |
| hIgG Control | 2.2001 | 2.0120 | 2.1457 | 1.8270 | 2.0417 | 1.9870 | 2.0126 | 2.3674 |

The results were shown in FIG. 14 and Table 24, and it was indicated in FIG. 24 that BLyS chimeric antibody could inhibit mouse B lymphocyte proliferation which was simulated by hBLyS-ECD.

TABLE 24
Inhibition of BLyS chimeric antibody on mouse B lmphocyte proliferation stimulated by hBLyS-ECD

| OD_{450 nm} | Antibody Conc. (nM) | | | | | | | | | |
|---|---|---|---|---|---|---|---|---|---|---|
| Clone No. | 100 | 20 | 4 | 0.8 | 0.16 | 0.032 | 0.0064 | 0.00128 | 0.000256 | 0 |
| c8E7D9C7F5 | 345.07 | 367.38 | 488.27 | 2069.27 | 4109.20 | 4616.25 | 5282.92 | 5154.99 | 4971.89 | 4754.75 |
| c97A3C2H4 | 443.52 | 423.67 | 577.74 | 1464.77 | 3739.53 | 4450.32 | 5280.16 | 4874.00 | 5015.22 | 4443.32 |
| c67A2E1D10 | 1103.30 | 2611.53 | 3496.52 | 3974.22 | 3896.76 | 4338.08 | 5169.07 | 4805.22 | 4720.71 | 4055.21 |
| c93C6F10D3 | 389.24 | 433.52 | 526.75 | 1091.97 | 4304.93 | 4521.70 | 4922.59 | 4515.87 | 4602.11 | 4315.42 |
| hIgG Control | 4884.43 | 4925.19 | 5096.35 | 5036.96 | 5366.47 | 5117.30 | 5066.07 | 4691.15 | 4943.82 | 4394.25 |

### Embodiment 8 Detection of In Vivo Neutralizing Activity of BLyS Antibody in Mouse

Female BALB/c mice (aged 8-9 weeks, purchased from Shanghai LinChang Systems Co., Ltd.) were feed under SPF conditions, and the experiment was started after one week of acclimatization. On day 1 and day 3 mice were injected intravenously at a dose of 3 mg/kg (1 hour before immunogen injection) with human or human-mouse chimeric BLyS monoclonal antibodies prepared in Embodiments 2 and 6 with clone numbers of 2-1G11, L1D12, c8E7D9C7F5, c20D1B6E9E5, c35E6F7C3, c97A3C2H4, c93C6F10D3 and c111D10D6G3. On day 1 to day 4 mice were stimulated daily by subcutaneous injection with the immunogen prepared in Embodiment 1 (hBLyS-ECD) at a dose of 0.3 mg/kg. On day 5 all animals were sacrificed, and the weight of mice spleen, the proportion of B lymphocytes to splenocytes, and concentration of IgA in serum were measured.

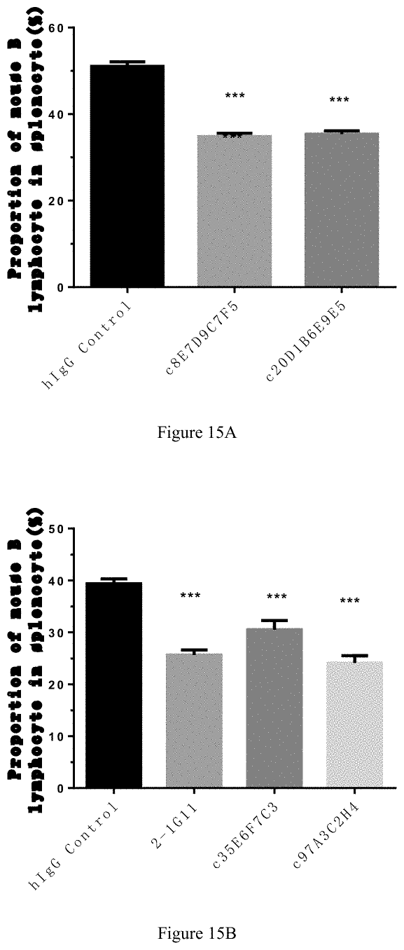

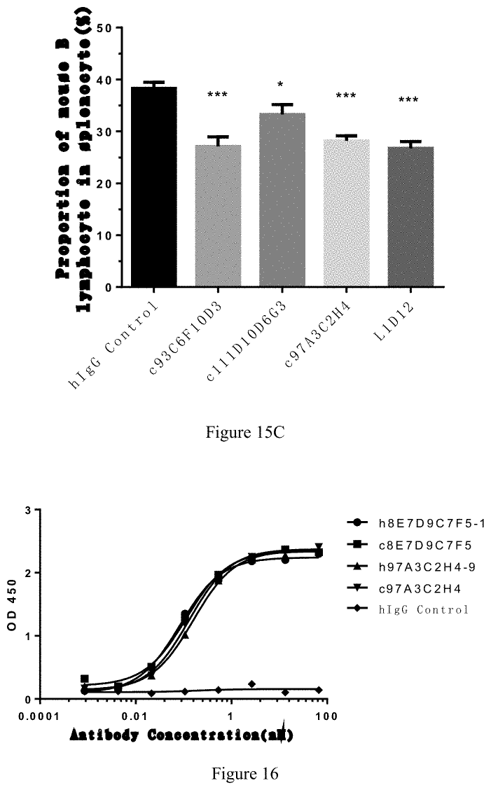

Some results were shown in FIGS. 15A-15C and Table 25. IgG control herein is human IgG. It was shown in the results that the BLyS antibody could reduce the increase proportion of B lymphocytes to mouse splenocytes which was stimulated by hBLyS-ECD.

TABLE 25-1
The effect of BLyS antibody on the proportion of mouse B
lymphocytes to splenocytes after hBLyS-ECD stimulation

| | Proportion of B lymphocytes |
|---|---|
| Clone No. | in splenocytes (%) |
| c8E7D9C7F5 | 34.80 |
| c20D1B6E9E5 | 35.36 |
| hIgG Control | 51.05 |

TABLE 25-2
The effect of BLyS antibody on the proportion of mouse B
lymphocytes to splenocytes after hBLyS-ECD stimulation

| | Proportion of B lymphocytes |
|---|---|
| Clone No. | in splenocytes (%) |
| 2-1G11 | 25.64 |
| c35E6F7C3 | 30.51 |
| c97A3C2H4 | 24.10 |
| hIgG Control | 39.36 |

TABLE 25-3
The effect of BLyS antibody on the proportion of mouse B
lymphocytes to splenocytes after hBLyS-ECD stimulation

| | Proportion of B lymphocytes |
|---|---|
| Clone No. | in splenocytes (%) |
| L1D12 | 26.75 |
| c93C6F10D3 | 27.11 |
| c111D10D6G3 | 33.24 |
| c97A3C2H4 | 28.19 |
| hIgG Control | 38.29 |

### Embodiment 9 Preparation and Identification of Humanized BLyS Antibody

The templates of human antibody heavy and light chain variable region that best match with the non-CDR regions of the above chimeric antibody c8E7D9C7F5 or c97A3C2H4 were selected from the Germline database. The sequences of the humanized BLyS antibody were selected from the sequences of human V_{H}, J_{H}, V_{K} and J_{K} exons. Among them, the template of the heavy chain variable region of antibody c8E7D9C7F5 is V_{H}1-18 of V_{H} exon and J_{H}-6 of J_{H} exon from a human antibody heavy chain, and the template of the light chain variable region is B3 of V_{K} exon, J_{K}-4 of the J_{K} exon from a human antibody light chain. The template of the heavy chain variable region of antibody c97A3C2H4 is V_{H}3-7 of V_{H} exon and J_{H}-6 of J_{H} exon from a human antibody heavy chain, and the template of the light chain variable region is A10 of V_{K} exon, J_{K}-4 of the J_{K} exon from a human antibody light chain.

The heavy chain CDRs and light chain CDRs of the chimeric antibody c8E7D9C7F5 or c97A3C2H4 determined according to Kabat definition were transplanted into the CDR regions of selected human templates, respectively. Then, a humanized antibody was obtained by reverse mutating buried residues, residues directly interacting with the CDR regions, and framework residues having a significant influence on the conformations of VH and VL based on the three-dimensional structure of mouse antibody.

The amino acid sequences alignment of heavy and light chain variable region of the humanized BLyS antibody variants and the heavy and light chain variable region of the chimeric antibody were shown in Table 26. The sequences of heavy chain variable region of the humanized BLyS antibody h8E7D9C7F5 variants were shown in SEQ ID No. 140, SEQ ID No. 141, SEQ ID No. 142, SEQ ID No. 143, SEQ ID No. 144, SEQ ID No. 144, No.145 and SEQ ID No.146, respectively, and the sequences of light chain variable region were shown in SEQ ID No.147 and SEQ ID No.148, respectively. The sequences of heavy chain variable region of the humanized BLyS antibody h97A3C2H4 variant were shown in SEQ ID No. 149, SEQ ID No. 150, SEQ ID No. 151 and SEQ ID No. 152, respectively, and the sequences of light chain variable region were shown in SEQ ID No.153, SEQ ID No.154, SEQ ID No.155, SEQ ID No.156, SEQ ID No.157, respectively.

Template of human heavy chain variable region
V_{H}1-18/J_{H}6 (SEQ ID NO. 136)
V_{H}1-18:
QVQLVQSGAEVKKPGASVKVSCKASGYTFTSYGISWVRQAPGQGLEWMGW
ISAYNGNTNYAQKLQGRVTMTTDTSTSTAYMELRSLRSDDTAVYYCAR;
J_{H}6:
WGQGTTVTVSS.
Template of human light chain variable region
B3/JK4 (SEQ ID NO. 137)
B3:
DIVMTQSPDSLAVSLGERATINCKSSQSVLYSSNNKNYLAWYQQKPGQPP
KLLIYWASTRESGVPDRFSGSGSGTDFTLTISSLQAEDVAVYYC;
J_{K}4:
FGGGTKVEIK.
Template of human heavy chain variable region
V_{H}3-7/JH6 (SEQ ID NO. 138)
V_{H}3-7:
EVQLVESGGGLVQPGGSLRLSCAASGFTFSSYWMSWVRQAPGKGLEWVAN
IKQDGSEKYYVDSVKGRFTISRDNAKNSLYLQMNSLRAEDTAVYYCAR;
J_{H}6:
WGQGTTVTVSS.
Template of human light chain variable region
A10/JK4 (SEQ ID NO. 139)
A10:
EIVLTQSPDFQSVTPKEKVTITCRASQSIGSSLHWYQQKPDQSPKLLIKY
ASQSFSGVPSRFSGSGSGTDFTLTINSLEAEDAATYYC;
J_{K}4:
FGGGTKVEIK.

TABLE 26-1
Sequence alignment of variable region of humanized BLyS antibody
h8E7D9C7F5 and variable region of chimeric antibody c8E7D9C7F5

| | | V_{H} | | | | V_{K} | | |
|---|---|---|---|---|---|---|---|---|
| | | template | | | | template | | |
| | | of heavy | | | | of light | | |
| | | chain | Donor | | | chain | Donor | |
| | | variable | framework | | | variable | framework | |
| | | region of | residues and | SEQ | | region of | residues and | SEQ |
| | Heavy | human | reverse | ID | Light | human | reverse | ID |
| Antibody | Chain | antibody | mutation | NO. | Chain | antibody | mutation | NO. |
| c8E7D9C7F5 | V_{H} | None | CDR | 33 | Vₖ | None | CDR | 37 |
| h8E7D9C7F5-1 | V_{H}.1 | V_{H}1-18/J_{H}6 | CDR-grafted, | 140 | Vₖ.1 | B3/J_{K}4 | CDR-grafted, | 147 |
| | | | Q1E | | | | Y49K | |
| h8E7D9C7F5-2 | V_{H}.1 | V_{H}1-18/J_{H}6 | CDR-grafted, | 140 | Vₖ.1a | B3/J_{K}4 | CDR-grafted, | 148 |
| | | | Q1E | | | | M4L, Y49K | |
| h8E7D9C7F5-3 | V_{H}.1a | V_{H}1-18/J_{H}6 | CDR-grafted, | 141 | Vₖ.1 | B3/J_{K}4 | CDR-grafted, | 147 |
| | | | Q1E, T71A | | | | Y49K | |
| h8E7D9C7F5-4 | V_{H}.1a | V_{H}1-18/J_{H}6 | CDR-grafted, | 141 | Vₖ.1a | B3/J_{K}4 | CDR-grafted, | 148 |
| | | | Q1E, T71A | | | | M4L, Y49K | |
| h8E7D9C7F5-5 | V_{H}.1b | V_{H}1-18/J_{H}6 | CDR-grafted, | 142 | Vₖ.1 | B3/J_{K}4 | CDR-grafted, | 147 |
| | | | Q1E, T71A, | | | | Y49K | |
| | | | M69L | | | | | |
| h8E7D9C7F5-6 | V_{H}.1b | V_{H}1-18/J_{H}6 | CDR-grafted, | 142 | Vₖ.1a | B3/J_{K}4 | CDR-grafted, | 148 |
| | | | Q1E, T71A, | | | | M4L, Y49K | |
| | | | M69L | | | | | |
| h8E7D9C7F5-7 | V_{H}.1c | V_{H}1-18/J_{H}6 | CDR-grafted, | 143 | Vₖ.1 | B3/J_{K}4 | CDR-grafted, | 147 |
| | | | Q1E, T71A, | | | | Y49K | |
| | | | M69L, M48I | | | | | |
| h8E7D9C7F5-8 | V_{H}.1c | V_{H}1-18/J_{H}6 | CDR-grafted, | 143 | Vₖ.1a | B3/J_{K}4 | CDR-grafted, | 148 |
| | | | Q1E, T71A, | | | | M4L, Y49K | |
| | | | M69L, M48I | | | | | |
| h8E7D9C7F5-9 | V_{H}.1d | V_{H}1-18/J_{H}6 | CDR-grafted, | 144 | Vₖ.1 | B3/J_{K}4 | CDR-grafted, | 147 |
| | | | Q1E, T71A, | | | | Y49K | |
| | | | M69L, M48I, | | | | | |
| | | | R38K | | | | | |
| h8E7D9C7F5-10 | V_{H}.1d | V_{H}1-18/J_{H}6 | CDR-grafted, | 144 | Vₖ.1a | B3/J_{K}4 | CDR-grafted, | 148 |
| | | | Q1E, T71A, | | | | M4L, Y49K | |
| | | | M69L, M48I, | | | | | |
| | | | R38K | | | | | |
| h8E7D9C7F5-11 | V_{H}.1e | V_{H}1-18/J_{H}6 | CDR-grafted, | 145 | V_{K}.1 | B3/J_{K}4 | CDR-grafted, | 147 |
| | | | Q1E, T71A, | | | | Y49K | |
| | | | M69L, M48I, | | | | | |
| | | | R38K, R66K, | | | | | |
| | | | V67A | | | | | |
| h8E7D9C7F5-12 | V_{H}.1e | V_{H}1-18/J_{H}6 | CDR-grafted, | 145 | Vₖ.1a | B3/J_{K}4 | CDR-grafted, | 148 |
| | | | Q1E, T71A, | | | | M4L, Y49K | |
| | | | M69L, M48I, | | | | | |
| | | | R38K, R66K, | | | | | |
| | | | V67A | | | | | |
| h8E7D9C7F5-13 | V_{H}.1f | V_{H}1-18/J_{H}6 | CDR-grafted, | 146 | Vₖ.1 | B3/J_{K}4 | CDR-grafted, | 147 |
| | | | Q1E, T71A, | | | | Y49K | |
| | | | M69L, M48I, | | | | | |
| | | | R38K, R66K, | | | | | |
| | | | V67A, G44A, | | | | | |
| | | | T73K, Y91F | | | | | |
| h8E7D9C7F5-14 | V_{H}.1f | V_{H}1-18/J_{H}6 | CDR-grafted, | 146 | Vₖ.1a | B3/J_{K}4 | CDR-grafted, | 148 |
| | | | Q1E, T71A, | | | | M4L, Y49K | |
| | | | M69L, M48I, | | | | | |
| | | | R38K, R66K, | | | | | |
| | | | V67A, G44A, | | | | | |
| | | | T73K, Y91F | | | | | |

TABLE 26-2
Sequence alignment of variable region of humanized BLyS antibody
h97A3C2H4 and variable region of chimeric antibody c97A3C2H4

| | | V_{H} | | | | V_{K} | | |
|---|---|---|---|---|---|---|---|---|
| | | template | | | | template | | |
| | | of heavy | | | | of light | | |
| | | chain | Donor | | | chain | Donor | |
| | | variable | framework | | | variable | framework | |
| | | region of | residues and | SEQ | | region of | residues and | SEQ |
| | Heavy | human | reverse | ID | Light | human | reverse | ID |
| Antibody | Chain | antibody | mutation | NO. | Chain | antibody | mutation | NO. |
| c97A3C2H4 | V_{H} | None | CDR | 73 | Vₖ | None | CDR | 77 |
| h97A3C2H4-1 | V_{H}.1 | V_{H}3-7/J_{H}6 | CDR-grafted | 149 | Vₖ.1 | A10/J_{K}4 | CDR-grafted | 153 |
| h97A3C2H4-2 | V_{H}.1 | V_{H}3-7/J_{H}6 | CDR-grafted | 149 | Vₖ.1a | A10/J_{K}4 | CDR-grafted, | 154 |
| | | | | | | | V58T | |
| h97A3C2H4-3 | V_{H}.1a | V_{H}3-7/J_{H}6 | CDR-grafted, | 150 | Vₖ.1 | A10/J_{K}4 | CDR-grafted | 153 |
| | | | A93T | | | | | |
| h97A3C2H4-4 | V_{H}.1a | V_{H}3-7/J_{H}6 | CDR-grafted, | 150 | Vₖ.1a | A10/J_{K}4 | CDR-grafted, | 154 |
| | | | A93T | | | | V58T | |
| h97A3C2H4-5 | V_{H}.1b | V_{H}3-7/J_{H}6 | CDR-grafted, | 151 | Vₖ.1 | A10/J_{K}4 | CDR-grafted | 153 |
| | | | A93T, G44R | | | | | |
| h97A3C2H4-6 | V_{H}.1b | V_{H}3-7/J_{H}6 | CDR-grafted, | 151 | Vₖ.1a | A10/J_{K}4 | CDR-grafted, | 154 |
| | | | A93T, G44R | | | | V58T | |
| h97A3C2H4-7 | V_{H}.1b | V_{H}3-7/J_{H}6 | CDR-grafted, | 151 | Vₖ.1b | A10/J_{K}4 | CDR-grafted, | 155 |
| | | | A93T, G44R | | | | V58T, | |
| | | | | | | | E1D, V3L | |
| h97A3C2H4-8 | V_{H}.1b | V_{H}3-7/J_{H}6 | CDR-grafted, | 151 | Vₖ.1c | A10/J_{K}4 | CDR-grafted, | 156 |
| | | | A93T, G44R | | | | V58T, | |
| | | | | | | | P40T | |
| h97A3C2H4-9 | V_{H}.1b | V_{H}3-7/J_{H}6 | CDR-grafted, | 151 | Vₖ.1d | A10/J_{K}4 | CDR-grafted, | 157 |
| | | | A93T, G44R | | | | V58T, | |
| | | | | | | | E1D, V3L, | |
| | | | | | | | P40T | |
| h97A3C2H4-10 | V_{H}.1c | V_{H}3-7/J_{H}6 | CDR-grafted, | 152 | Vₖ.1b | A10/J_{K}4 | CDR-grafted, | 155 |
| | | | A93T, G44R, | | | | V58T, | |
| | | | G42E | | | | E1D, V3L | |
| h97A3C2H4-11 | V_{H}.1c | V_{H}3-7/J_{H}6 | CDR-grafted, | 152 | Vₖ.1c | A10/J_{K}4 | CDR-grafted, | 156 |
| | | | A93T, G44R, | | | | V58T, | |
| | | | G42E | | | | P40T | |
| h97A3C2H4-12 | V_{H}.1c | V_{H}3-7/J_{H}6 | CDR-grafted, | 152 | Vₖ.1d | A10/J_{K}4 | CDR-grafted, | 157 |
| | | | A93T, G44R, | | | | V58T, | |
| | | | G42E | | | | E1D, V3L, | |
| | | | | | | | P40T | |

The nucleotide sequences of heavy and light chain variable region of the humanized BLyS antibody variants were shown in Table 27. The nucleotide sequences of the heavy chain variable region of the humanized BLyS antibody h8E7D9C7F5 variant were shown in SEQ ID No. 158, SEQ ID No. 159, SEQ ID No. 160, SEQ ID No. 161, SEQ ID No. 162, SEQ ID No.163, SEQ ID No.164, respectively, and the nucleotide sequences of the light chain variable region were shown in SEQ ID No.165 and SEQ ID No.166, respectively. The nucleotide sequences of heavy chain variable region of the humanized BLyS antibody h97A3C2H4 variants were shown in SEQ ID No. 167, SEQ ID No. 168, SEQ ID No. 169, SEQ ID No. 170, respectively, and the nucleotide sequences of light chain variable region were shown in SEQ ID No. 171, SEQ ID No. 172, SEQ ID No. 173, SEQ ID No. 174, SEQ ID No. 175, respectively.

TABLE 27-1
Nucleotide SEQ ID NO. of variable region
of humanized BLyS antibody h8E7D9C7F5

| | Heavy Chain | Light Chain |
|---|---|---|
| | variable | variable |
| Antibody | region | region |
| h8E7D9C7F5-1 | 158 | 165 |
| h8E7D9C7F5-2 | 158 | 166 |
| h8E7D9C7F5-3 | 159 | 165 |
| h8E7D9C7F5-4 | 159 | 166 |
| h8E7D9C7F5-5 | 160 | 165 |
| h8E7D9C7F5-6 | 160 | 166 |
| h8E7D9C7F5-7 | 161 | 165 |
| h8E7D9C7F5-8 | 161 | 166 |
| h8E7D9C7F5-9 | 162 | 165 |
| h8E7D9C7F5-10 | 162 | 166 |
| h8E7D9C7F5-11 | 163 | 165 |
| h8E7D9C7F5-12 | 163 | 166 |
| h8E7D9C7F5-13 | 164 | 165 |
| h8E7D9C7F5-14 | 164 | 166 |

TABLE 27-2
Nucleotide SEQ ID NO. of variable region
of humanized BLyS antibody h97A3C2H4

| | Heavy Chain | Light Chain |
|---|---|---|
| | variable | variable |
| Antibody | region | region |
| h97A3C2H4-1 | 167 | 171 |
| h97A3C2H4-2 | 167 | 172 |
| h97A3C2H4-3 | 168 | 171 |
| h97A3C2H4-4 | 168 | 172 |
| h97A3C2H4-5 | 169 | 171 |
| h97A3C2H4-6 | 169 | 172 |
| h97A3C2H4-7 | 169 | 173 |
| h97A3C2H4-8 | 169 | 174 |
| h97A3C2H4-9 | 169 | 175 |
| h97A3C2H4-10 | 170 | 173 |
| h97A3C2H4-11 | 170 | 174 |
| h97A3C2H4-12 | 170 | 175 |

Each domain was assembled using overlap extension PCR using overlapping oligonucleotides synthesized from humanized V_{H} or V_{L} domains. The nucleotide sequences of humanized V_{H} and V_{L} domains were synthesized. The V_{H} domain was directionally cloned into the expression vector comprising the signal peptide and the heavy chain IgG1 constant region of human antibody using the restriction sites incorporated into the PCR product (wherein the expression vector was purchased from Invitrogen and the recombination process was performed by Shanghai ChemPartner Co., Ltd.), the V_{L} domain was directionally cloned into the expression vector comprising the signal peptide and light chain kappa constant region of human antibody (wherein the expression vector was purchased from Invitrogen and the recombination process was performed by Shanghai ChemPartner Co., Ltd.). The harvested recombinant plasmid was confirmed by sequencing, then high purity of recombinant plasmid was extracted and filtered through a 0.22 m filter membrane for following transfection. Production and preparation of humanized BLyS antibody referred to step (iii) of Embodiment 2, and the harvested BLyS antibody was characterized (see Embodiments 3-5 and 7).

The results were shown in FIG. 16 and Table 28, and it was shown in Table 28 that the humanized antibody could bind to BLyS recombinant protein in ELISA assay. Data in the table is the OD value at 450 nm.

TABLE 28
ELISA detection of the binding of humanized
BLyS antibody to biotinylated hBLvS-ECD

| OD_{450 nm} | Antibody Conc. (nM) | | | | | | | |
|---|---|---|---|---|---|---|---|---|
| Clone No. | 66.7 | 13.3 | 2.67 | 0.534 | 0.107 | 0.0213 | 0.00427 | 0 |
| h8E7D9C7F5-1 | 2.2986 | 2.2019 | 2.1808 | 1.8797 | 1.3505 | 0.4991 | 0.1950 | 0.1285 |
| c8E7D9C7F5 | 2.3233 | 2.3700 | 2.2093 | 1.9660 | 1.2801 | 0.5083 | 0.1978 | 0.3222 |
| h97A3C2H4-9 | 2.3711 | 2.2738 | 2.2379 | 1.8774 | 1.0192 | 0.3716 | 0.1794 | 0.1978 |
| c97A3C2H4 | 2.4051 | 2.3430 | 2.2493 | 1.9071 | 1.1888 | 0.3745 | 0.1937 | 0.1653 |
| hIgG Control | 0.1401 | 0.1035 | 0.2374 | 0.1414 | 0.1171 | 0.0887 | 0.1212 | 0.1194 |

The binding and dissociation between antibody and antigen was detected by Biacore. The results were shown in Table 29, indicating that the humanized BLyS antibody has high affinity for hBLyS-ECD.

TABLE 29
Affinity constant of humanized BLyS antibody for hBLyS-ECD

| | Affinity | Binding | Dissociation |
|---|---|---|---|
| | Constant | Constant | Constant |
| Clone No. | K_{D} (nM) | kₐ (1/Ms) | k_{d} (1/s) |
| c8E7D9C7F5 | 2.941 | 2.427 × 10⁵ | 7.138 × 10⁻⁴ |
| h8E7D9C7F5-1 | 4.329 | 2.177 × 10⁵ | 9.423 × 10⁻⁴ |
| c97A3C2H4 | 1.488 | 1.206 × 10⁵ | 1.794 × 10⁻⁴ |
| h97A3C2H4-9 | 1.878 | 1.150 × 10⁵ | 2.160 × 10⁻⁴ |

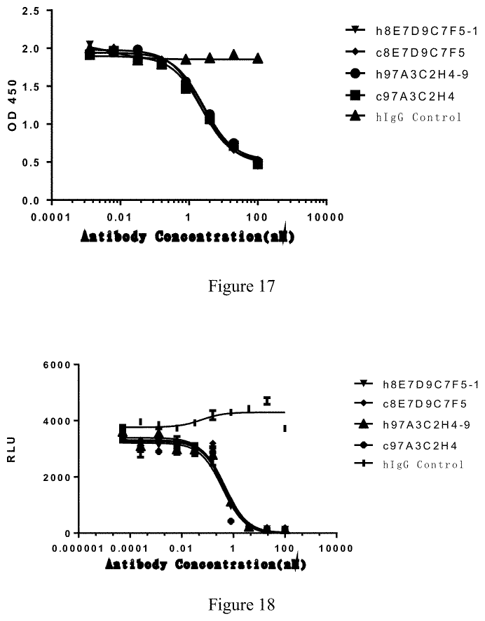

The results were shown in FIG. 17 and Table 30. Table 30 showed that the humanized BLyS antibody could block the binding of BLyS protein to its receptor BAFF R. The data in the table is OD₄₅₀ₙₘ value.

TABLE 30
The binding of BLyS protein to its receptor BAFF
R that blocked by humanized BLyS antibody

| OD_{450 nm} | Antibody Conc. (nM) | | | | | | | |
|---|---|---|---|---|---|---|---|---|
| Clone No. | 100 | 20 | 4 | 0.8 | 0.16 | 0.032 | 0.0064 | 0 |
| h8E7D9C7F5-1 | 0.4501 | 0.7213 | 1.1150 | 1.5421 | 1.7835 | 1.8727 | 1.9808 | 2.0499 |
| c8E7D9C7F5 | 0.5296 | 0.6604 | 1.0354 | 1.5597 | 1.8533 | 1.9748 | 2.0080 | 1.9519 |
| h97A3C2H4-9 | 0.5060 | 0.7477 | 1.1276 | 1.5564 | 1.8094 | 1.9797 | 1.9601 | 1.9430 |
| c97A3C2H4 | 0.4740 | 0.7166 | 1.0687 | 1.4683 | 1.7871 | 1.8425 | 1.9609 | 1.9144 |
| hIgG Control | 1.8655 | 1.9140 | 1.8696 | 1.8512 | 1.7914 | 1.8435 | 1.9850 | 1.9992 |

The results were shown in FIG. 18 and Table 31. It was shown in Table 31 that humanized BLyS antibody could inhibit the proliferation of mouse B lymphocytes that stimulated by hBLyS-ECD.

TABLE 31
Inhibition of humanized BLyS antibody on mouse B lmphocyte proliferation stimulated by hBLyS-ECD

| OD_{450 nm} | Antibody Conc. (nM) | | | | | | | | | |
|---|---|---|---|---|---|---|---|---|---|---|
| Clone No. | 100 | 20 | 4 | 0.8 | 0.16 | 0.032 | 0.0064 | 0.00128 | 0.000256 | 0 |
| h8E7D9C7F5-1 | 131.23 | 151.51 | 208.78 | 950.84 | 2726.07 | 2973.03 | 3199.70 | 3120.96 | 2995.69 | 3481.26 |
| c8E7D9C7F5 | 116.00 | 187.02 | 187.02 | 408.36 | 3195.83 | 2949.63 | 3021.83 | 3357.99 | 3305.91 | 3639.69 |
| h97A3C2H4-9 | 126.73 | 137.39 | 216.75 | 1108.61 | 2772.70 | 2964.58 | 2965.76 | 3541.38 | 3084.20 | 3591.13 |
| c97A3C2H4 | 169.37 | 158.71 | 222.67 | 428.76 | 2892.33 | 2964.58 | 3053.41 | 2900.62 | 3107.89 | 3451.37 |
| hIgG Control | 3721.21 | 4701.60 | 4430.12 | 4291.42 | 4201.33 | 3921.55 | 3733.06 | 3861.09 | 3967.79 | 3573.02 |

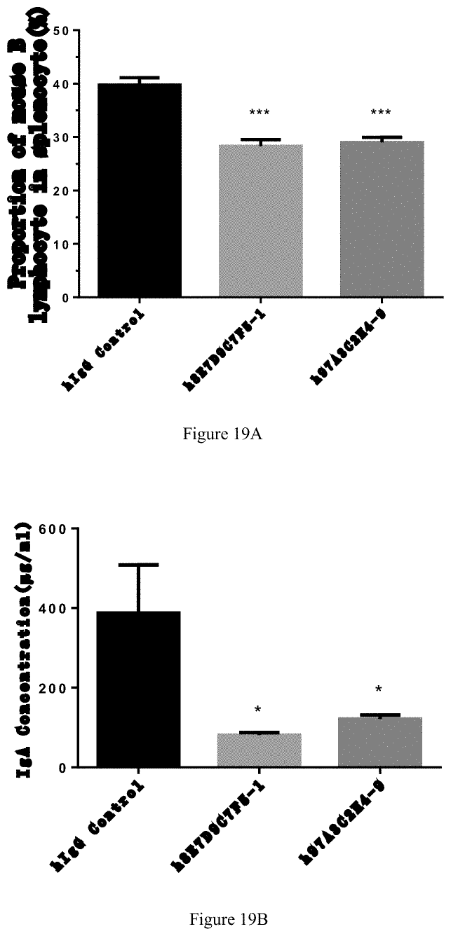

Meanwhile, in vivo neutralizing activity of BLyS antibody was detected in mouse. The results were shown in FIGS. 19A-19B and Table 32. It was shown in the results that humanized BLyS antibody could reduce the increase proportion of B lymphocytes to mouse splenocytes stimulated by hBLyS-ECD and effectively inhibit the level of IgA in serum at the same time.

TABLE 32-1
The effect of humanized BLyS antibody on the proportion of mouse
B lymphocytes to splenocytes after hBLyS-ECD stimulation

| | Proportion of B lymphocytes |
|---|---|
| Clone No. | in splenocytes (%) |
| h8E7D9C7F5-1 | 28.29 |
| h97A3C2H4-9 | 28.98 |
| hIgG Control | 39.74 |

TABLE 32-2
Effect of humanized BLyS antibody
on IgA concentration in mouse serum

| | IgA Conc. |
|---|---|
| Clone No. | (μg/mL) |
| h8E7D9C7F5-1 | 80.89 |
| h97A3C2H4-9 | 121.33 |
| hIgG Control | 387.02 |

All references mentioned in the present invention are incorporated herein by reference, as if each reference was individually incorporated by reference. In addition, it is to be understood that after reading the foregoing contents of the present invention, those skilled in the art can make various changes or modifications to the present invention, and these equivalent forms also fall within the scope of the appended claims of the present application.

What is claimed is:

1. An isolated protein, wherein said protein comprises complementary determining regions (CDRs) of BLyS antibody: one or more of the heavy chain CDR1, heavy chain CDR2 and heavy chain CDR3, and/or one or more of the light chain CDR1, light chain CDR2, and light chain CDR3, wherein the heavy chain CDR1 contains amino acid sequences represented by SEQ ID No.2, SEQ ID No.10, SEQ ID No.18, SEQ ID No.26, SEQ ID No.34, SEQ ID No.42, SEQ ID No.50, SEQ ID No.58, SEQ ID No.66, SEQ ID No.74, SEQ ID No.82, SEQ ID No.90 or SEQ ID No.98; the heavy chain CDR2 contains amino acid sequences represented by SEQ ID No.3, SEQ ID No.11, SEQ ID No.19, SEQ ID No.27, SEQ ID No.35, SEQ ID No.43, SEQ ID No.51, SEQ ID No.59, SEQ ID No.67, SEQ ID No.75, SEQ ID No.83, SEQ ID No.91 or SEQ ID No.99; the heavy chain CDR3 contains amino acid sequences represented by SEQ ID No.4, SEQ ID No.12, SEQ ID No.20, SEQ ID No.28, SEQ ID No.36, SEQ ID No.44, SEQ ID No.52, SEQ ID No.60, SEQ ID No.68, SEQ ID No.76, SEQ ID No.84, SEQ ID No.92 or SEQ ID No.100; the light chain CDR1 contains amino acid sequences represented by SEQ ID No.6, SEQ ID No.14, SEQ ID No.22, SEQ ID No.30, SEQ ID No.38, SEQ ID No.46, SEQ ID No.54, SEQ ID No.62, SEQ ID No.70, SEQ ID No.78, SEQ ID No.86, SEQ ID No.94 or SEQ ID No.102; the light chain CDR2 contains amino acid sequences represented by SEQ ID No.7, SEQ ID No.15, SEQ ID No.23, SEQ ID No.31, SEQ ID No.39, SEQ ID No.47, SEQ ID No.55, SEQ ID No.63, SEQ ID No.71, SEQ ID No.79, SEQ ID No.87, SEQ ID No.95 or SEQ ID No.103; the light chain CDR3 contains amino acid sequences represented by SEQ ID No.8, SEQ ID No.16, SEQ ID No.24, SEQ ID No.32, SEQ ID No.40, SEQ ID No.48, SEQ ID No.56, SEQ ID No.64, SEQ ID No.72, SEQ ID No.80, SEQ ID No.88, SEQ ID No.96 or SEQ ID No.104;
Or, the amino acid sequences of the heavy chain CDR1 is at least 80% identical to the amino acid sequences represented by SEQ ID No.2, SEQ ID No.10, SEQ ID No.18, SEQ ID No.26, SEQ ID No.34, SEQ ID No.42, SEQ ID No.50, SEQ ID No.58, SEQ ID No.66, SEQ ID No.74, SEQ ID No.82, SEQ ID No.90 or SEQ ID No.98; the amino acid sequences of the heavy chain CDR2 is at least 80% identical to the amino acid sequences represented by SEQ ID No.3, SEQ ID No.11, SEQ ID No.19, SEQ ID No.27, SEQ ID No.35, SEQ ID No.43, SEQ ID No.51, SEQ ID No.59, SEQ ID No.67, SEQ ID No.75, SEQ ID No.83, SEQ ID No.91 or SEQ ID No.99; the amino acid sequences of the heavy chain CDR3 is at least 80% identical to the amino acid sequences represented by SEQ ID No.4, SEQ ID No.12, SEQ ID No.20, SEQ ID No.28, SEQ ID No.36, SEQ ID No.44, SEQ ID No.52, SEQ ID No.60, SEQ ID No.68, SEQ ID No.76, SEQ ID No.84, SEQ ID No.92 or SEQ ID No.100; the amino acid sequences of the light chain CDR1 is at least 80% identical to the amino acid sequences represented by SEQ ID No.6, SEQ ID No.14, SEQ ID No.22, SEQ ID No.30, SEQ ID No.38, SEQ ID No.46, SEQ ID No.54, SEQ ID No.62, SEQ ID No.70, SEQ ID No.78, SEQ ID No.86, SEQ ID No.94 or SEQ ID No.102; the amino acid sequences of the light chain CDR2 is at least 80% identical to the amino acid sequences represented by SEQ ID No.7, SEQ ID No.15, SEQ ID No.23, SEQ ID No.31, SEQ ID No.39, SEQ ID No.47, SEQ ID No.55, SEQ ID No.63, SEQ ID No.71, SEQ ID No.79, SEQ ID No.87, SEQ ID No.95 or SEQ ID No.103; the amino acid sequences of the light chain CDR3 is at least 80% identical to the amino acid sequences represented by SEQ ID No.8, SEQ ID No.16, SEQ ID No.24, SEQ ID No.32, SEQ ID No.40, SEQ ID No.48, SEQ ID No.56, SEQ ID No.64, SEQ ID No.72, SEQ ID No.80, SEQ ID No.88, SEQ ID No.96 or SEQ ID No.104.

2. The protein of claim 1, wherein a heavy chain CDR1 comprises amino acid sequence of SEQ ID No.2, a heavy chain CDR2 comprises amino acid sequence of SEQ ID No.3 and a heavy chain CDR3 comprises amino acid sequence of SEQ ID No.4; a heavy chain heavy chain CDR1 comprises amino acid sequence of SEQ ID No.10, a heavy chain CDR2 comprises amino acid sequence of SEQ ID No.11 and a CDR3 comprises amino acid sequence of SEQ ID No.12; a heavy chain CDR1 comprises amino acid sequence of SEQ ID No.18, a heavy chain CDR2 comprises amino acid sequence of SEQ ID No.19 and a heavy chain CDR3 comprises amino acid sequence of SEQ ID No.20; a heavy chain CDR1 comprises amino acid sequence of SEQ ID No.26, a heavy chain CDR2 comprises amino acid sequence of SEQ ID No.27 and a heavy chain CDR3 comprises amino acid sequence of SEQ ID No.28; a heavy chain CDR1 comprises amino acid sequence of SEQ ID No.34, a heavy chain CDR2 comprises amino acid sequence of SEQ ID No.35 and a CDR3 comprises amino acid sequence of SEQ ID No.36; a heavy chain CDR1 comprises amino acid sequence of SEQ ID No.42, a heavy chain CDR2 comprises amino acid sequence of SEQ ID No.43 and a heavy chain CDR3 comprises amino acid sequence of SEQ ID No.44; a heavy chain CDR1 comprises amino acid sequence of SEQ ID No.50, a heavy chain CDR2 comprises amino acid sequence of SEQ ID No.51 and a heavy chain CDR3 comprises amino acid sequence of SEQ ID No.52; a heavy chain CDR1 comprises amino acid sequence of SEQ ID No.58, a heavy chain CDR2 comprises amino acid sequence of SEQ ID No.59 and a heavy chain CDR3 comprises amino acid sequence of SEQ ID No.60; a heavy chain CDR1 comprises amino acid sequence of SEQ ID No.66, a heavy chain CDR2 comprises amino acid sequence of SEQ ID No.67 and a heavy chain CDR3 comprises amino acid sequence of SEQ ID No.68; a heavy chain CDR1 comprises amino acid sequence of SEQ ID No.74, a heavy chain CDR2 comprises amino acid sequence of SEQ ID No.75 and a heavy chain CDR3 comprises amino acid sequence of SEQ ID No.76; a heavy chain CDR1 comprises amino acid sequence of SEQ ID No.82, a heavy chain CDR2 comprises amino acid sequence of SEQ ID No.83 and a heavy chain CDR3 comprises amino acid sequence of SEQ ID No.84; a heavy chain CDR1 comprises amino acid sequence of SEQ ID No.90, a heavy chain CDR2 comprises amino acid sequence of SEQ ID No.91 and a heavy chain CDR3 comprises amino acid sequence of SEQ ID No.92; a heavy chain CDR1 comprises amino acid sequence of SEQ ID No.98, a CDR2 comprises amino acid sequence of SEQ ID No.99 and a heavy chain CDR3 comprises amino acid sequence of SEQ ID No.100;
a light chain CDR1 comprises amino acid sequence of SEQ ID No.6, a light chain CDR2 comprises amino acid sequence of SEQ ID No.7 and a light chain CDR3 comprises amino acid sequence of SEQ ID No.8; a light chain CDR1 comprises amino acid sequence of SEQ ID No.14, a light chain CDR2 comprises amino acid sequence of SEQ ID No.15 and a light chain CDR3 comprises amino acid sequence of SEQ ID No.16; a light chain CDR1 comprises amino acid sequence of SEQ ID No.22, a light chain CDR2 comprises amino acid sequence of SEQ ID No.23 and a light chain CDR3 comprises amino acid sequence of SEQ ID No.24; a light chain CDR1 comprises amino acid sequence of SEQ ID No.30, a light chain CDR2 comprises amino acid sequence of SEQ ID No.31 and a light chain CDR3 comprises amino acid sequence of SEQ ID No.32; a light chain CDR1 comprises amino acid sequence of SEQ ID No.38, a light chain CDR2 comprises amino acid sequence of SEQ ID No.39 and a light chain CDR3 comprises amino acid sequence of SEQ ID No.40; a light chain CDR1 comprises amino acid sequence of SEQ ID No.46, a light chain CDR2 comprises amino acid sequence of SEQ ID No.47 and a light chain CDR3 comprises amino acid sequence of SEQ ID No.48; a light chain CDR1 comprises amino acid sequence of SEQ ID No.54, a light chain CDR2 comprises amino acid sequence of SEQ ID No.55 and a light chain CDR3 comprises amino acid sequence of SEQ ID No.56; a light chain CDR1 comprises amino acid sequence of SEQ ID No.62, a light chain CDR2 comprises amino acid sequence of SEQ ID No.63 and a light chain CDR3 comprises amino acid sequence of SEQ ID No.64; a light chain CDR1 comprises amino acid sequence of SEQ ID No.70, a light chain CDR2 comprises amino acid sequence of SEQ ID No.71 and a light chain CDR3 comprises amino acid sequence of SEQ ID No.72; a light chain CDR1 comprises amino acid sequence of SEQ ID No.78, a light chain CDR2 comprises amino acid sequence of SEQ ID No.79 and a light chain CDR3 comprises amino acid sequence of SEQ ID No.80; a light chain CDR1 comprises amino acid sequence of SEQ ID No.86, a light chain CDR2 comprises amino acid sequence of SEQ ID No.87 and a light chain CDR3 comprises amino acid sequence of SEQ ID No.88; a light chain CDR1 comprises amino acid sequence of SEQ ID No.94, a light chain CDR2 comprises amino acid sequence of SEQ ID No.95 and a CDR3 comprises amino acid sequence of SEQ ID No.96; or, a light chain light chain CDR1 comprises amino acid sequence of SEQ ID No.102, a light chain CDR2 comprises amino acid sequence of SEQ ID No.103 and a light chain CDR3 comprises amino acid sequence of SEQ ID No.104.

3. The protein of claim 2, wherein said protein further comprises a framework region of BLyS antibody, the framework region comprises a framework region of heavy chain and/or a framework region of light chain.

4. The protein of claim 3, wherein said protein comprises the variable region of BLyS antibody heavy chain and/or variable region of BLyS antibody light chain forming by CDR and framework, wherein the amino acid sequences of heavy chain variable region are represented by SEQ ID No.1, SEQ ID No.9, SEQ ID No.17, SEQ ID No.25, SEQ ID No.33, SEQ ID No.41, SEQ ID No.49, SEQ ID No.57, SEQ ID No.65, SEQ ID No.73, SEQ ID No.81, SEQ ID No.89, SEQ ID No.97, SEQ ID NO. 140, SEQ ID NO. 141, SEQ ID NO. 142, SEQ ID NO. 143, SEQ ID NO. 144, SEQ ID NO. 145, SEQ ID NO. 146, SEQ ID NO. 149, SEQ ID NO. 150, SEQ ID NO. 151 or SEQ ID NO. 152; the amino acid sequences of light chain variable region are represented by SEQ ID No.5, SEQ ID No.13, SEQ ID No.21, SEQ ID No.29, SEQ ID No.37, SEQ ID No.45, SEQ ID No.53, SEQ ID No.61, SEQ ID No.69, SEQ ID No.77, SEQ ID No.85, SEQ ID No.93, SEQ ID No.101, SEQ ID No.147, SEQ ID No.148, SEQ ID No.153, SEQ ID No.154, SEQ ID No.155, SEQ ID No.156 or SEQ ID No.157.

5. The protein of claim 4, wherein an amino acid sequence of the heavy chain variable region is represented by SEQ ID No.1 and an amino acid sequence of the light chain variable region is represented by SEQ ID No.5; an amino acid sequence of the heavy chain variable region is represented by SEQ ID No.9 and an amino acid sequence of the light chain variable region is represented by SEQ ID No.13; an amino acid sequence of the heavy chain variable region is represented by SEQ ID No.17 and an amino acid sequence of the light chain variable region is represented by SEQ ID No.21; an amino acid sequence of the heavy chain variable region is represented by SEQ ID No.25 and an amino acid sequence of the light chain variable region is represented by SEQ ID No.29; an amino acid sequence of the heavy chain variable region is represented by SEQ ID No.33 and an amino acid sequence of the light chain variable region is represented by SEQ ID No.37; an amino acid sequence of the heavy chain variable region is represented by SEQ ID No.41 and an amino acid sequence of the light chain variable region is represented by SEQ ID No.45; an amino acid sequence of the heavy chain variable region is represented by SEQ ID No.49 and an amino acid sequence of the light chain variable region is represented by SEQ ID No.53; an amino acid sequence of the heavy chain variable region is represented by SEQ ID No.57 and an amino acid sequence of the light chain variable region is represented by SEQ ID No.61; an amino acid sequence of the heavy chain variable region is represented by SEQ ID No.65 and an amino acid sequence of the light chain variable region is represented by SEQ ID No.69; an amino acid sequence of the heavy chain variable region is represented by SEQ ID No.73 and an amino acid sequence of the light chain variable region is represented by SEQ ID No.77; an amino acid sequence of the heavy chain variable region is represented by SEQ ID No.81 and an amino acid sequence of the light chain variable region is represented by SEQ ID No.85; an amino acid sequence of the heavy chain variable region is represented by SEQ ID No.89 and an amino acid sequence of the light chain variable region is represented by SEQ ID No.93; an amino acid sequence of the heavy chain variable region is represented by SEQ ID No.97 and an amino acid sequence of the light chain variable region is represented by SEQ ID No.101; an amino acid sequence of the heavy chain variable region is represented by SEQ ID No.140 and an amino acid sequence of the light chain variable region is represented by SEQ ID No.147; an amino acid sequence of the heavy chain variable region is represented by SEQ ID No.140 and an amino acid sequence of the light chain variable region is represented by SEQ ID No.148; an amino acid sequence of the heavy chain variable region is represented by SEQ ID No.141 and an amino acid sequence of the light chain variable region is represented by SEQ ID No.147; an amino acid sequence of the heavy chain variable region is represented by SEQ ID No.141 and an amino acid sequence of the light chain variable region is represented by SEQ ID No.148; an amino acid sequence of the heavy chain variable region is represented by SEQ ID No.142 and an amino acid sequence of the light chain variable region is represented by SEQ ID No.147; an amino acid sequence of the heavy chain variable region is represented by SEQ ID No.142 and an amino acid sequence of the light chain variable region is represented by SEQ ID No.148; an amino acid sequence of the heavy chain variable region is represented by SEQ ID No.143 and an amino acid sequence of the light chain variable region is represented by SEQ ID No.147; an amino acid sequence of the heavy chain variable region is represented by SEQ ID No.143 and an amino acid sequence of the light chain variable region is represented by SEQ ID No.148; an amino acid sequence of the heavy chain variable region is represented by SEQ ID No.144 and an amino acid sequence of the light chain variable region is represented by SEQ ID No.147; an amino acid sequence of the heavy chain variable region is represented by SEQ ID No.144 and an amino acid sequence of the light chain variable region is represented by SEQ ID No.148; an amino acid sequence of the heavy chain variable region is represented by SEQ ID No.145 and an amino acid sequence of the light chain variable region is represented by SEQ ID No.147; an amino acid sequence of the heavy chain variable region is represented by SEQ ID No.145 and an amino acid sequence of the light chain variable region is represented by SEQ ID No.148; a heavy chain variable region is represented by SEQ ID No.146 and an amino acid sequence of the light chain variable region is represented by SEQ ID No.147; an amino acid sequence of the heavy chain variable region is represented by SEQ ID No.146 and an amino acid sequence of the light chain variable region is represented by SEQ ID No.148; an amino acid sequence of the heavy chain variable region is represented by SEQ ID No.149 and an amino acid sequence of the light chain variable region is represented by SEQ ID No.153; an amino acid sequence of the heavy chain variable region is represented by SEQ ID No.149 and an amino acid sequence of the light chain variable region is represented by SEQ ID No.154; an amino acid sequence of the heavy chain variable region is represented by SEQ ID No.150 and an amino acid sequence of the light chain variable region is represented by SEQ ID No.153; an amino acid sequence of the heavy chain variable region is represented by SEQ ID No.150 and an amino acid sequence of the light chain variable region is represented by SEQ ID No.154; an amino acid sequence of the heavy chain variable region is represented by SEQ ID No.151 and an amino acid sequence of the light chain variable region is represented by SEQ ID No.153; an amino acid sequence of the heavy chain variable region is represented by SEQ ID No.151 and an amino acid sequence of the light chain variable region is represented by SEQ ID No.154; an amino acid sequence of the heavy chain variable region is represented by SEQ ID No.151 and an amino acid sequence of the light chain variable region is represented by SEQ ID No.155; an amino acid sequence of the heavy chain variable region is represented by SEQ ID No.151 and an amino acid sequence of the light chain variable region is represented by SEQ ID No.156; an amino acid sequence of the heavy chain variable region is represented by SEQ ID No.151 and an amino acid sequence of the light chain variable region is represented by SEQ ID No.157; an amino acid sequence of the heavy chain variable region is represented by SEQ ID No.152 and an amino acid sequence of the light chain variable region is represented by SEQ ID No.155; an amino acid sequence of the heavy chain variable region is represented by SEQ ID No.152 and an amino acid sequence of the light chain variable region is represented by SEQ ID No.156; an amino acid sequence of the heavy chain variable region is represented by SEQ ID No.152 and an amino acid sequence of the light chain variable region is represented by SEQ ID No.157.

6. The protein of claim 5, wherein said protein further comprises heavy chain constant region of antibody and/or light chain constant region of antibody.

7. The protein of claim 6, wherein the heavy chain constant region of antibody is a heavy chain constant region of human or mouse antibody, the light chain constant region of antibody is a light chain constant region of human or mouse antibody.

8. The protein of claim 7, wherein the heavy chain constant region of antibody is a heavy chain constant region of human antibody, the light chain constant region of antibody is a light chain constant region of human antibody.

9. The protein of claim 5, wherein the protein is monoclonal antibody, full-length antibody protein, antibody-antigen binding domain protein fragment, bispecific antibody, multispecific antibody, single-chain antibody fragment, single-domain or single-region antibody of BLyS antibody.

10. A nucleic acid, wherein said nucleic acid encodes the protein of claim 9.

11. The nucleic acid of claim 10, wherein the nucleotide sequences of the nucleic acid encoding the heavy chain variable region are shown in SEQ ID No.105, SEQ ID No.107, SEQ ID No.109, SEQ ID No.111, SEQ ID No.113, SEQ ID No.115, SEQ ID No.117, SEQ ID No.119, SEQ ID No.121, SEQ ID No.123, SEQ ID No.125, SEQ ID No.127, SEQ ID No.129, SEQ ID NO. 158, SEQ ID NO. 159, SEQ ID NO. 160, SEQ ID NO. 161, SEQ ID NO. 162, SEQ ID NO. 163, SEQ ID NO. 164, SEQ ID NO. 167, SEQ ID NO. 168, SEQ ID NO. 169 or SEQ ID NO. 170; and/or, the nucleotide sequences of the nucleic acid encoding the light chain variable region are shown in SEQ ID No.106, SEQ ID No.108, SEQ ID No.110, SEQ ID No.112, SEQ ID No.114, SEQ ID No.116, SEQ ID No.118, SEQ ID No.120, SEQ ID No.122, SEQ ID No.124, SEQ ID No.126, SEQ ID No.128, SEQ ID No.130, SEQ ID No.165, SEQ ID No.166, SEQ ID No.171, SEQ ID No.172, SEQ ID No.173, SEQ ID No.174 or SEQ ID No.175.

12. The nucleic acid of claim 11, wherein a nucleotide sequence of the nucleic acid encoding the heavy chain variable region is shown in SEQ ID No.105, and a nucleotide sequence of the nucleic acid encoding the light chain variable region is shown in SEQ ID No.106; a nucleotide sequence of the nucleic acid encoding the heavy chain variable region is shown in SEQ ID No.107, and a nucleotide sequence of the nucleic acid encoding the light chain variable region is shown in SEQ ID No.108; a nucleotide sequence of the nucleic acid encoding the heavy chain variable region is shown in SEQ ID No.109, and a nucleotide sequence of the nucleic acid encoding the light chain variable region is shown in SEQ ID No.110; a nucleotide sequence of the nucleic acid encoding the heavy chain variable region is shown in SEQ ID No.111, and a nucleotide sequence of the nucleic acid encoding the light chain variable region is shown in SEQ ID No.112; a nucleotide sequence of the nucleic acid encoding the heavy chain variable region is shown in SEQ ID No.113, and a nucleotide sequence of the nucleic acid encoding the light chain variable region is shown in SEQ ID No.114; a nucleotide sequence of the nucleic acid encoding the heavy chain variable region is shown in SEQ ID No.115, and a nucleotide sequence of the nucleic acid encoding the light chain variable region is shown in SEQ ID No.116; a nucleotide sequence of the nucleic acid encoding the heavy chain variable region is shown in SEQ ID No.117, and a nucleotide sequence of the nucleic acid encoding the light chain variable region is shown in SEQ ID No.118; a nucleotide sequence of the nucleic acid encoding the heavy chain variable region is shown in SEQ ID No.119, and a nucleotide sequence of the nucleic acid encoding the light chain variable region is shown in SEQ ID No.120; a nucleotide sequence of the nucleic acid encoding the heavy chain variable region is shown in SEQ ID No.121, and a nucleotide sequence of the nucleic acid encoding the light chain variable region is shown in SEQ ID No.122; a nucleotide sequence of the nucleic acid encoding the heavy chain variable region is shown in SEQ ID No.123, and a nucleotide sequence of the nucleic acid encoding the light chain variable region is shown in SEQ ID No.124; a nucleotide sequence of the nucleic acid encoding the heavy chain variable region is shown in SEQ ID No.125, and a nucleotide sequence of the nucleic acid encoding the light chain variable region is shown in SEQ ID No.126; a nucleotide sequence of the nucleic acid encoding the heavy chain variable region is shown in SEQ ID No.127, and a nucleotide sequence of the nucleic acid encoding the light chain variable region is shown in SEQ ID No.128; a nucleotide sequence of the nucleic acid encoding the heavy chain variable region is shown in SEQ ID No.129, and a nucleotide sequence of the nucleic acid encoding the light chain variable region is shown in SEQ ID No.130; a nucleotide sequence of the nucleic acid encoding the heavy chain variable region is shown in SEQ ID No.158, and a nucleotide sequence of the nucleic acid encoding the light chain variable region is shown in SEQ ID No.165; a nucleotide sequence of the nucleic acid encoding the heavy chain variable region is shown in SEQ ID No.158, and a nucleotide sequence of the nucleic acid encoding the light chain variable region is shown in SEQ ID No.166; a nucleotide sequence of the nucleic acid encoding the heavy chain variable region is shown in SEQ ID No.159, and a nucleotide sequence of the nucleic acid encoding the light chain variable region is shown in SEQ ID No.165; a nucleotide sequence of the nucleic acid encoding the heavy chain variable region is shown in SEQ ID No.159, and a nucleotide sequence of the nucleic acid encoding the light chain variable region is shown in SEQ ID No.166; a nucleotide sequence of the nucleic acid encoding the heavy chain variable region is shown in SEQ ID No.160, and a nucleotide sequence of the nucleic acid encoding the light chain variable region is shown in SEQ ID No.165; a nucleotide sequence of the nucleic acid encoding the heavy chain variable region is shown in SEQ ID No.160, and a nucleotide sequence of the nucleic acid encoding the light chain variable region is shown in SEQ ID No.166; a nucleotide sequence of the nucleic acid encoding the heavy chain variable region is shown in SEQ ID No.161, and a nucleotide sequence of the nucleic acid encoding the light chain variable region is shown in SEQ ID No.165; a nucleotide sequence of the nucleic acid encoding the heavy chain variable region is shown in SEQ ID No.161, and a nucleotide sequence of the nucleic acid encoding the light chain variable region is shown in SEQ ID No.166; a nucleotide sequence of the nucleic acid encoding the heavy chain variable region is shown in SEQ ID No.162, and a nucleotide sequence of the nucleic acid encoding the light chain variable region is shown in SEQ ID No.165; a nucleotide sequence of the nucleic acid encoding the heavy chain variable region is shown in SEQ ID No.162, and a nucleotide sequence of the nucleic acid encoding the light chain variable region is shown in SEQ ID No.166; a nucleotide sequence of the nucleic acid encoding the heavy chain variable region is shown in SEQ ID No.163, and a nucleotide sequence of the nucleic acid encoding the light chain variable region is shown in SEQ ID No.165; a nucleotide sequence of the nucleic acid encoding the heavy chain variable region is shown in SEQ ID No.163, and a nucleotide sequence of the nucleic acid encoding the light chain variable region is shown in SEQ ID No.166; a nucleotide sequence of the nucleic acid encoding the heavy chain variable region is shown in SEQ ID No.164, and a nucleotide sequence of the nucleic acid encoding the light chain variable region is shown in SEQ ID No.165; a nucleotide sequence of the nucleic acid encoding the heavy chain variable region is shown in SEQ ID No.164, and a nucleotide sequence of the nucleic acid encoding the light chain variable region is shown in SEQ ID No.166; a nucleotide sequence of the nucleic acid encoding the heavy chain variable region is shown in SEQ ID No.167, and a nucleotide sequence of the nucleic acid encoding the light chain variable region is shown in SEQ ID No.171; a nucleotide sequence of the nucleic acid encoding the heavy chain variable region is shown in SEQ ID No.167, and a nucleotide sequence of the nucleic acid encoding the light chain variable region is shown in SEQ ID No.172; a nucleotide sequence of the nucleic acid encoding the heavy chain variable region is shown in SEQ ID No.168, and a nucleotide sequence of the nucleic acid encoding the light chain variable region is shown in SEQ ID No.171; a nucleotide sequence of the nucleic acid encoding the heavy chain variable region is shown in SEQ ID No.168, and a nucleotide sequence of the nucleic acid encoding the light chain variable region is shown in SEQ ID No.172; a nucleotide sequence of the nucleic acid encoding the heavy chain variable region is shown in SEQ ID No.169, and a nucleotide sequence of the nucleic acid encoding the light chain variable region is shown in SEQ ID No.171; a nucleotide sequence of the nucleic acid encoding the heavy chain variable region is shown in SEQ ID No.169, and a nucleotide sequence of the nucleic acid encoding the light chain variable region is shown in SEQ ID No.172; a nucleotide sequence of the nucleic acid encoding the heavy chain variable region is shown in SEQ ID No.169, and a nucleotide sequence of the nucleic acid encoding the light chain variable region is shown in SEQ ID No.173; a nucleotide sequence of the nucleic acid encoding the heavy chain variable region is shown in SEQ ID No.169, and a nucleotide sequence of the nucleic acid encoding the light chain variable region is shown in SEQ ID No.174; a nucleotide sequence of the nucleic acid encoding the heavy chain variable region is shown in SEQ ID No.169, and a nucleotide sequence of the nucleic acid encoding the light chain variable region is shown in SEQ ID No.175; a nucleotide sequence of the nucleic acid encoding the heavy chain variable region is shown in SEQ ID No.170, and a nucleotide sequence of the nucleic acid encoding the light chain variable region is shown in SEQ ID No.173; a nucleotide sequence of the nucleic acid encoding the heavy chain variable region is shown in SEQ ID No.170, and a nucleotide sequence of the nucleic acid encoding the light chain variable region is shown in SEQ ID No.174; a nucleotide sequence of the nucleic acid encoding the heavy chain variable region is shown in SEQ ID No.170, and a nucleotide sequence of the nucleic acid encoding the light chain variable region is shown in SEQ ID No.175.

13. A recombinant expression vector comprising the nucleic acid of claim 12.

14. A recombinant expression transformant comprising the recombinant expression vector of claim 13.

15. A method for preparing a BLyS antibody, comprising the steps: culture the recombinant expression transformant of claim 14, and harvest the BLyS antibody from the culture.

16. A method for detecting BLyS protein-overexpressing cells, comprising the steps: contact a protein according to claim with a sample to be tested in vitro, and detect the combination between the protein and the sample to be tested.

17. A composition for detecting BLyS protein-overexpressing cells, wherein said composition comprises the protein according to claim 9 as an active ingredient.

18. A pharmaceutical composition, wherein said composition comprises the protein according to claim 9 as an active ingredient and a pharmaceutically acceptable carrier.

19. The pharmaceutical composition of claim 18, wherein said pharmaceutical composition comprises 0.01-99.99% of the protein according to claim 9 and 0.01-99.99% of pharmaceutical carrier, the percentage is the mass percentage of the pharmaceutical composition.

20. A process for preventing or treating diseases associated with abnormal expression or dysfunction of BLyS in a subject in need thereof, comprising: administering an effective amount of the protein according to claim 9 or the pharmaceutical composition according to claim 19 to the subject.


SEQUENCE LISTING (187 sequences)

SEQ ID NO: 1 - protein, 125 aa, Artificial Sequence
  Description of Artificial Sequence Synthetic polypeptide
EVQLVESRGGVVQPGRSLRLSCAASGFTFSSYGMHWVRQAPGKGLEWVAVISYDGSNKYYADSVKGRFTISRDNSKNTLYLQMNSLRAEDAAVYYCARDRVDILTGYSIYGMDVWGQGTTVTVSS

SEQ ID NO: 2 - protein, 10 aa, Artificial Sequence
  Description of Artificial Sequence Synthetic peptide
GFTFSSYGMH

SEQ ID NO: 3 - protein, 17 aa, Artificial Sequence
  Description of Artificial Sequence Synthetic peptide
VISYDGSNKYYADSVKG

SEQ ID NO: 4 - protein, 16 aa, Artificial Sequence
  Description of Artificial Sequence Synthetic peptide
DRVDILTGYSIYGMDV

SEQ ID NO: 5 - protein, 107 aa, Homo sapiens
EIVMTQSPATLSLSPGERATLSCRASQSVSSYLAWYQQKPGQAPRLLIYDASKRATGIPARFSGSGSGTDFTLTISSLEPEDFAVYYCQQRSNWPLTFGGGTKVEIK

SEQ ID NO: 6 - protein, 11 aa, Artificial Sequence
  Description of Artificial Sequence Synthetic peptide
RASQSVSSYLA

SEQ ID NO: 7 - protein, 7 aa, Artificial Sequence
  Description of Artificial Sequence Synthetic peptide
DASKRAT

SEQ ID NO: 8 - protein, 9 aa, Artificial Sequence
  Description of Artificial Sequence Synthetic peptide
QQRSNWPLT

SEQ ID NO: 9 - protein, 125 aa, Artificial Sequence
  Description of Artificial Sequence Synthetic polypeptide
QIQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARTYYDILTGYYYYGMDVWGQGTTVTVSS

SEQ ID NO: 10 - protein, 10 aa, Artificial Sequence
  Description of Artificial Sequence Synthetic peptide
GYTFTSYGIS

SEQ ID NO: 11 - protein, 17 aa, Artificial Sequence
  Description of Artificial Sequence Synthetic peptide
WISAYNGNTNYAQKLQG

SEQ ID NO: 12 - protein, 16 aa, Artificial Sequence
  Description of Artificial Sequence Synthetic peptide
TYYDILTGYYYYGMDV

SEQ ID NO: 13 - protein, 107 aa, Artificial Sequence
  Description of Artificial Sequence Synthetic polypeptide
EIVLTQSPGTLSLSPGERATLSCRTSQSVDTYLAWYQQKPGQAPRLLIYDASNRATGIPARFSGSGSGTDFTLTISSLEPEDFAVYYCQQRSNWPLTFGGGTKVEIK

SEQ ID NO: 14 - protein, 11 aa, Artificial Sequence
  Description of Artificial Sequence Synthetic peptide
RTSQSVDTYLA

SEQ ID NO: 15 - protein, 7 aa, Artificial Sequence
  Description of Artificial Sequence Synthetic peptide
DASNRAT

SEQ ID NO: 16 - protein, 9 aa, Artificial Sequence
  Description of Artificial Sequence Synthetic peptide
QQRSNWPLT

SEQ ID NO: 17 - protein, 121 aa, Artificial Sequence
  Description of Artificial Sequence Synthetic polypeptide
QVQLVQSGGGLVQPGGSLRLSCAASGFTVSSNYMSWVRQAPGKGLEWVSVIYSGGSTYYADSVKGRFTISRDNSKNTLYLQMNSLRAEDTAVYYCARGFHDILTGYGMDVWGQGTTVTVSS

SEQ ID NO: 18 - protein, 10 aa, Artificial Sequence
  Description of Artificial Sequence Synthetic peptide
GFTVSSNYMS

SEQ ID NO: 19 - protein, 16 aa, Artificial Sequence
  Description of Artificial Sequence Synthetic peptide
VIYSGGSTYYADSVKG

SEQ ID NO: 20 - protein, 13 aa, Artificial Sequence
  Description of Artificial Sequence Synthetic peptide
GFHDILTGYGMDV

SEQ ID NO: 21 - protein, 109 aa, Artificial Sequence
  Description of Artificial Sequence Synthetic polypeptide
SSELTQDPAVSVALGQTVRITCQGDSLRNYYAGWYQQKPGQAPVLVIYGKNNRPSGIPDRFSGSSSGNTASLTITGTQAEDEADYYCSSRDNSGTHLVEFGGGTKLTVL

SEQ ID NO: 22 - protein, 11 aa, Artificial Sequence
  Description of Artificial Sequence Synthetic peptide
QGDSLRNYYAG

SEQ ID NO: 23 - protein, 7 aa, Artificial Sequence
  Description of Artificial Sequence Synthetic peptide
GKNNRPS

SEQ ID NO: 24 - protein, 12 aa, Artificial Sequence
  Description of Artificial Sequence Synthetic peptide
SSRDNSGTHLVE

SEQ ID NO: 25 - protein, 118 aa, Artificial Sequence
  Description of Artificial Sequence Synthetic polypeptide
EVQLQQSGAELVKPGASVKLSCTAAGFNIQDTYIHWVKQRPEQGLEWIGRIDPANANTVYDPKFQGKATITADTSSNTAYLQVSSLTSEDTAVYYCASPVITAGFAYWGQGTLVTVSA

SEQ ID NO: 26 - protein, 10 aa, Artificial Sequence
  Description of Artificial Sequence Synthetic peptide
GFNIQDTYIH

SEQ ID NO: 27 - protein, 17 aa, Artificial Sequence
  Description of Artificial Sequence Synthetic peptide
RIDPANANTVYDPKFQG

SEQ ID NO: 28 - protein, 9 aa, Artificial Sequence
  Description of Artificial Sequence Synthetic peptide
PVITAGFAY

SEQ ID NO: 29 - protein, 106 aa, Artificial Sequence
  Description of Artificial Sequence Synthetic polypeptide
QIVLTQSPAIMSASPGEKVTMTCGASSSVNYVHWYQQKSGTSPKRWIYDTSKLASGVPARFSGSGSGTSYSLTISSMEAEDAATYYCQQWTRSPLTFGAGTKVELK

SEQ ID NO: 30 - protein, 10 aa, Artificial Sequence
  Description of Artificial Sequence Synthetic peptide
GASSSVNYVH

SEQ ID NO: 31 - protein, 7 aa, Artificial Sequence
  Description of Artificial Sequence Synthetic peptide
DTSKLAS

SEQ ID NO: 32 - protein, 9 aa, Artificial Sequence
  Description of Artificial Sequence Synthetic peptide
QQWTRSPLT

SEQ ID NO: 33 - protein, 125 aa, Artificial Sequence
  Description of Artificial Sequence Synthetic polypeptide
QVQLQQSGVELARPGASVKLSCKASGYTFTSYGISWVKQRTGQALEWIGEIYPRSGNTYYNEKFKGKATLTADKSSSTAYMELRSLTSEDSAVYFCARSGPITTLVATDYGMDYWGQGTSVTVSS

SEQ ID NO: 34 - protein, 10 aa, Artificial Sequence
  Description of Artificial Sequence Synthetic peptide
GYTFTSYGIS

SEQ ID NO: 35 - protein, 17 aa, Artificial Sequence
  Description of Artificial Sequence Synthetic peptide
EIYPRSGNTYYNEKFKG

SEQ ID NO: 36 - protein, 16 aa, Artificial Sequence
  Description of Artificial Sequence Synthetic peptide
SGPITTLVATDYGMDY

SEQ ID NO: 37 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKFASNLESGVPARFSGSGSGTDFTLNIHPVEEEDAATYYCQHSREFPYTFGGGTKLEIK

SEQ ID NO: 38 - protein, 15 aa, Artificial Sequence
  Description of Artificial Sequence Synthetic peptide
RASKSVSTSSYSYMH

SEQ ID NO: 39 - protein, 7 aa, Artificial Sequence
  Description of Artificial Sequence Synthetic peptide
FASNLES

SEQ ID NO: 40 - protein, 9 aa, Artificial Sequence
  Description of Artificial Sequence Synthetic peptide
QHSREFPYT

SEQ ID NO: 41 - protein, 124 aa, Artificial Sequence
  Description of Artificial Sequence Synthetic polypeptide
EVQLQQSVAELVRPGASVKLSCTASDFNIKNTYIHWVKQRPEQGLEWIGRIDPVNGNTKYAPKFQDKATIIVDTYSNTAYLQLSSLASEDTAVYYCARWPITTIVATDWYFDVWGTGTTVTVSS

SEQ ID NO: 42 - protein, 10 aa, Artificial Sequence
  Description of Artificial Sequence Synthetic peptide
DFNIKNTYIH

SEQ ID NO: 43 - protein, 17 aa, Artificial Sequence
  Description of Artificial Sequence Synthetic peptide
RIDPVNGNTKYAPKFQD

SEQ ID NO: 44 - protein, 15 aa, Artificial Sequence
  Description of Artificial Sequence Synthetic peptide
WPITTIVATDWYFDV

SEQ ID NO: 45 - protein, 112 aa, Artificial Sequence
  Description of Artificial Sequence Synthetic polypeptide
DVLMTQTQLALPVRLGEQASISCRSSQSIVNSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 46 - protein, 16 aa, Artificial Sequence
  Description of Artificial Sequence Synthetic peptide
RSSQSIVNSNGNTYLE

SEQ ID NO: 47 - protein, 7 aa, Artificial Sequence
  Description of Artificial Sequence Synthetic peptide
KVSNRFS

SEQ ID NO: 48 - protein, 7 aa, Artificial Sequence
  Description of Artificial Sequence Synthetic peptide
FQGSHVP

SEQ ID NO: 49 - protein, 116 aa, Artificial Sequence
  Description of Artificial Sequence Synthetic polypeptide
QVQLQQSGAELVRPGTSVKISCKASGYAFTNYWLGWVKQRPGHGLEWIGDIYPGSGNTYYNEKFKGKATLTADKSSSTAYMQLSSLTSEDSAVFFCAAYLYAMDYWGQGTSVTVSS

SEQ ID NO: 50 - protein, 7 aa, Artificial Sequence
  Description of Artificial Sequence Synthetic peptide
GYAFTNY

SEQ ID NO: 51 - protein, 17 aa, Artificial Sequence
  Description of Artificial Sequence Synthetic peptide
DIYPGSGNTYYNEKFKG

SEQ ID NO: 52 - protein, 7 aa, Artificial Sequence
  Description of Artificial Sequence Synthetic peptide
YLYAMDY

SEQ ID NO: 53 - protein, 106 aa, Artificial Sequence
  Description of Artificial Sequence Synthetic polypeptide
QIVLTQSPAIMSASPGEKVTITCSASSSVSYMHWFQQKPGTSPKLWIYSASNLASGVPARFSGSGSGTSYSLTISRMEAEDAATYYCQQRGSYPFTFGSGTKLEIK

SEQ ID NO: 54 - protein, 10 aa, Artificial Sequence
  Description of Artificial Sequence Synthetic peptide
SASSSVSYMH

SEQ ID NO: 55 - protein, 7 aa, Artificial Sequence
  Description of Artificial Sequence Synthetic peptide
SASNLAS

SEQ ID NO: 56 - protein, 9 aa, Artificial Sequence
  Description of Artificial Sequence Synthetic peptide
QQRGSYPFT

SEQ ID NO: 57 - protein, 120 aa, Artificial Sequence
  Description of Artificial Sequence Synthetic polypeptide
QVQLKESGPGLVAPSQSLSITCTVSGLSLNSHDISWIRQPPGKGLEWLGVIWSGGGTKYNSAFMSRLSISKDNSKSQVFLKMNSLHTDDTAIYYCVRTPSMITYSAMDYWGQGTSVTVSS

SEQ ID NO: 58 - protein, 10 aa, Artificial Sequence
  Description of Artificial Sequence Synthetic peptide
GLSLNSHDIS

SEQ ID NO: 59 - protein, 16 aa, Artificial Sequence
  Description of Artificial Sequence Synthetic peptide
VIWSGGGTKYNSAFMS

SEQ ID NO: 60 - protein, 12 aa, Artificial Sequence
  Description of Artificial Sequence Synthetic peptide
TPSMITYSAMDY

SEQ ID NO: 61 - protein, 108 aa, Artificial Sequence
  Description of Artificial Sequence Synthetic polypeptide
EIVLTQSPALMAASPGEKVTITCSVSSSISSRNLHWYQQKSQTSPKAWIHATSNLASGVPVRFSGSGSGTSYSLTISSMEAEDAATYYCQQWSSYPLTFGSGTKLEIK

SEQ ID NO: 62 - protein, 12 aa, Artificial Sequence
  Description of Artificial Sequence Synthetic peptide
SVSSSISSRNLH

SEQ ID NO: 63 - protein, 7 aa, Artificial Sequence
  Description of Artificial Sequence Synthetic peptide
ATSNLAS

SEQ ID NO: 64 - protein, 9 aa, Artificial Sequence
  Description of Artificial Sequence Synthetic peptide
QQWSSYPLT

SEQ ID NO: 65 - protein, 118 aa, Artificial Sequence
  Description of Artificial Sequence Synthetic polypeptide
EVQLQQSGPELVKSGASVKISCKASGYTFTDHKMHWVKQSHGKSLEWIGYISPYNGGTGYNQKFKSKVTLTVDISSSTAYMELRSLTSEDSAVYYCARWGFYDVMDSWGQGTSVTVSS

SEQ ID NO: 66 - protein, 10 aa, Artificial Sequence
  Description of Artificial Sequence Synthetic peptide
GYTFTDHKMH

SEQ ID NO: 67 - protein, 17 aa, Artificial Sequence
  Description of Artificial Sequence Synthetic peptide
YISPYNGGTGYNQKFKS

SEQ ID NO: 68 - protein, 9 aa, Artificial Sequence
  Description of Artificial Sequence Synthetic peptide
WGFYDVMDS

SEQ ID NO: 69 - protein, 112 aa, Artificial Sequence
  Description of Artificial Sequence Synthetic polypeptide
DVLLTQTPLSLPVSLGDQASISCRSSQTIVHIIGDTYLEWYLQKPGQSPKLLIYKVSNRFSGVPDRFSGSGAGTDFTLKISRVEAEDLGVYYCFQGSHVPRTFGGGTKLEIK

SEQ ID NO: 70 - protein, 16 aa, Artificial Sequence
  Description of Artificial Sequence Synthetic peptide
RSSQTIVHIIGDTYLE

SEQ ID NO: 71 - protein, 7 aa, Artificial Sequence
  Description of Artificial Sequence Synthetic peptide
KVSNRFS

SEQ ID NO: 72 - protein, 9 aa, Artificial Sequence
  Description of Artificial Sequence Synthetic peptide
FQGSHVPRT

SEQ ID NO: 73 - protein, 120 aa, Artificial Sequence
  Description of Artificial Sequence Synthetic polypeptide
EVKLVESGGGLVQPGGSLKLSCAASGFTFSTYTMSWVRQTPEKRLEWVAYISYGGGSTSYPDTVKGRFTISRDNAKNTLYLQMSSLKSEDTAIYYCTRHVTVVENSMDYWGQGTSVTVSS

SEQ ID NO: 74 - protein, 10 aa, Artificial Sequence
  Description of Artificial Sequence Synthetic peptide
GFTFSTYTMS

SEQ ID NO: 75 - protein, 17 aa, Artificial Sequence
  Description of Artificial Sequence Synthetic peptide
YISYGGGSTSYPDTVKG

SEQ ID NO: 76 - protein, 11 aa, Artificial Sequence
  Description of Artificial Sequence Synthetic peptide
HVTVVENSMDY

SEQ ID NO: 77 - protein, 107 aa, Artificial Sequence
  Description of Artificial Sequence Synthetic polypeptide
DILLTQSPDILSVSPGERVSFSCRASQSIGTSIHWYQQRTNGSPRLLIKYVSESFSGTPSRFSGSGSGTAFTLTINSVESEDIADYYCQQTHSWPWTFGGGTKLEIK

SEQ ID NO: 78 - protein, 11 aa, Artificial Sequence
  Description of Artificial Sequence Synthetic peptide
RASQSIGTSIH

SEQ ID NO: 79 - protein, 7 aa, Artificial Sequence
  Description of Artificial Sequence Synthetic peptide
YVSESFS

SEQ ID NO: 80 - protein, 9 aa, Artificial Sequence
  Description of Artificial Sequence Synthetic peptide
QQTHSWPWT

SEQ ID NO: 81 - protein, 122 aa, Artificial Sequence
  Description of Artificial Sequence Synthetic polypeptide
EVQLQQSGAELVKPGASVKLSCTASGFNIKNYYMHWVKQRTEEGLEWIGRIDPEDGETKYAPKFQAKATITADTSSNTAYLQLSSLTSEDTAVYFCARKGYYGNYGAWFAYWGQGTLVTVSA

SEQ ID NO: 82 - protein, 10 aa, Artificial Sequence
  Description of Artificial Sequence Synthetic peptide
GFNIKNYYMH

SEQ ID NO: 83 - protein, 17 aa, Artificial Sequence
  Description of Artificial Sequence Synthetic peptide
RIDPEDGETKYAPKFQA

SEQ ID NO: 84 - protein, 13 aa, Artificial Sequence
  Description of Artificial Sequence Synthetic peptide
KGYYGNYGAWFAY

SEQ ID NO: 85 - protein, 107 aa, Artificial Sequence
  Description of Artificial Sequence Synthetic polypeptide
DIQMTQSPASLSVSVGETVTITCRASEDLYSNLAWYQQKQGKSPQLLVYAATFLADGVPSRFSGSGSGTQYSLKINSLQSEDFGNYYCQHFWGTPPTFGGGTKLEIK

SEQ ID NO: 86 - protein, 11 aa, Artificial Sequence
  Description of Artificial Sequence Synthetic peptide
RASEDLYSNLA

SEQ ID NO: 87 - protein, 7 aa, Artificial Sequence
  Description of Artificial Sequence Synthetic peptide
AATFLAD

SEQ ID NO: 88 - protein, 9 aa, Artificial Sequence
  Description of Artificial Sequence Synthetic peptide
QHFWGTPPT

SEQ ID NO: 89 - protein, 114 aa, Artificial Sequence
  Description of Artificial Sequence Synthetic polypeptide
QVQLQQPGAELVKPGASVKMSCKASGYTFTRHWITWVKQRPGQGLEWIGDIFPGSGSINYNEKFKSEATLTVEKSSSTAYMQLSSLTSEDSAVYYCARGDVDVWGTGTTVTVSS

SEQ ID NO: 90 - protein, 7 aa, Artificial Sequence
  Description of Artificial Sequence Synthetic peptide
GYTFTRH

SEQ ID NO: 91 - protein, 17 aa, Artificial Sequence
  Description of Artificial Sequence Synthetic peptide
DIFPGSGSINYNEKFKS

SEQ ID NO: 92 - protein, 5 aa, Artificial Sequence
  Description of Artificial Sequence Synthetic peptide
GDVDV

SEQ ID NO: 93 - protein, 107 aa, Artificial Sequence
  Description of Artificial Sequence Synthetic polypeptide
DIQMTQSPSSLSASLGERVSLTCRASQEIRGYLSWLQQKPNGTIKRLIYAASTLDSGVPKRFSATRSGSDFSLTISSLESEDFADYYCLQYASYPFTFGSGTKLEIK

SEQ ID NO: 94 - protein, 11 aa, Artificial Sequence
  Description of Artificial Sequence Synthetic peptide
RASQEIRGYLS

SEQ ID NO: 95 - protein, 7 aa, Artificial Sequence
  Description of Artificial Sequence Synthetic peptide
AASTLDS

SEQ ID NO: 96 - protein, 9 aa, Artificial Sequence
  Description of Artificial Sequence Synthetic peptide
LQYASYPFT

SEQ ID NO: 97 - protein, 123 aa, Artificial Sequence
  Description of Artificial Sequence Synthetic polypeptide
EVQLVESGGGLVTPGGSLKLSCVASGFAFSTYGMSWVRQTPERRLEWVAYISGGGGSIYYPDSVKGRFTISRDNAKNILYLQMSSLKSEDTAMYHCTRHPDLLLRYDYALDHWGQGTSVTVSS

SEQ ID NO: 98 - protein, 10 aa, Artificial Sequence
  Description of Artificial Sequence Synthetic peptide
GFAFSTYGMS

SEQ ID NO: 99 - protein, 17 aa, Artificial Sequence
  Description of Artificial Sequence Synthetic peptide
YISGGGGSIYYPDSVKG

SEQ ID NO: 100 - protein, 14 aa, Artificial Sequence
  Description of Artificial Sequence Synthetic peptide
HPDLLLRYDYALDH

SEQ ID NO: 101 - protein, 107 aa, Artificial Sequence
  Description of Artificial Sequence Synthetic polypeptide
DIQMTQSPASLSASVGETVTITCRASENIHIYLAWYQQKQGKSPQLLVYNGKTLADGVPSRFSGSGSGTQYSLKINTLQPEDFGNYYCQHFWNSTWTFGGGTKLEIK

SEQ ID NO: 102 - protein, 11 aa, Artificial Sequence
  Description of Artificial Sequence Synthetic peptide
RASENIHIYLA

SEQ ID NO: 103 - protein, 7 aa, Artificial Sequence
  Description of Artificial Sequence Synthetic peptide
NGKTLAD

SEQ ID NO: 104 - protein, 9 aa, Artificial Sequence
  Description of Artificial Sequence Synthetic peptide
QHFWNSTWT

SEQ ID NO: 105 - DNA, 375 bp, Artificial Sequence
  Description of Artificial Sequence Synthetic polynucleotide
gaggtgcagctggtggagtcccggggaggcgtggtccagcctgggaggtccctgagactctcctgtgcagcgtctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacgcggccgtgtattactgtgcgagagatcgggtagatattttgactggttattctatctacggtatggacgtctggggccaagggaccacggtcaccgtctcctca

SEQ ID NO: 106 - DNA, 321 bp, Artificial Sequence
  Description of Artificial Sequence Synthetic polynucleotide
gaaattgtgatgactcagtctccagccaccctgtctttgtctccaggggaaagagccaccctctcctgcagggccagtcagagtgttagcagctacttagcctggtaccaacagaaacctggccaggctcccaggctcctcatctatgatgcatccaagagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 107 - DNA, 375 bp, Artificial Sequence
  Description of Artificial Sequence Synthetic polynucleotide
caaatccagctggtacaatctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaacgtattacgatattttgactggttattactactacggtatggacgtctggggccaagggaccacggtcaccgtctcctca

SEQ ID NO: 108 - DNA, 321 bp, Artificial Sequence
  Description of Artificial Sequence Synthetic polynucleotide
gaaattgtgttgacgcagtctccaggcaccctgtctttgtctccaggggagagagccaccctctcctgcaggaccagtcagagtgttgacacctacttggcctggtaccaacagaagcctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccactcactttcggcggagggaccaaggtggagatcaaa

SEQ ID NO: 109 - DNA, 363 bp, Artificial Sequence
  Description of Artificial Sequence Synthetic polynucleotide
caggtccagctggtgcagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatctgcaaatgaacagcctgagagctgaggacacggctgtgtattactgtgcgagaggtttccacgatattttgactggttacggtatggacgtctggggccaagggaccacggtcaccgtctcctca

SEQ ID NO: 110 - DNA, 327 bp, Artificial Sequence
  Description of Artificial Sequence Synthetic polynucleotide
tcttctgagctgactcaggaccctgctgtgtctgtggccttgggacagacagtcaggatcacatgccaaggagacagcctcagaaactattatgcaggctggtaccaacagaagccaggacaggcccctgtacttgtcatctatggaaagaacaaccggccctcaggaatcccagaccgattctctggctccagctcaggaaacacagcttccttgaccatcactgggactcaggcggaagatgaggctgactattactgtagctcccgggacaacagtggtacccatctcgtggaattcggcggagggaccaagttgaccgtccta

SEQ ID NO: 111 - DNA, 354 bp, Artificial Sequence
  Description of Artificial Sequence Synthetic polynucleotide
gaggttcagctgcagcagtctggggcagagcttgtgaagccaggggcctcagtcaagttgtcctgcacagctgctggcttcaacattcaagacacctatattcattgggtgaagcagaggcctgagcagggcctggagtggattggaaggattgatcctgcgaatgctaacactgtatatgacccgaagttccagggcaaggccactataacagcagacacatcctccaacacagcctacctgcaggtcagcagcctgacatctgaggacactgccgtctattactgtgccagtcccgttattactgcgggatttgcttactggggccaagggactctggtcactgtctctgca

SEQ ID NO: 112 - DNA, 318 bp, Artificial Sequence
  Description of Artificial Sequence Synthetic polynucleotide
caaattgttctcacccagtctccagcaatcatgtctgcttctccaggggagaaggtcaccatgacctgcggtgccagctcaagtgtaaattacgtgcactggtaccagcagaagtcagggacctcccccaaaagatggatttatgacacatccaaattggcttctggagtccctgctcgcttcagtggcagtgggtctgggacctcttactctctcacaatcagcagcatggaggctgaagatgctgccacttattactgccagcagtggactagatccccgctcacgttcggtgctgggaccaaggtggagctgaaa

SEQ ID NO: 113 - DNA, 375 bp, Artificial Sequence
  Description of Artificial Sequence Synthetic polynucleotide
caggttcagctgcagcagtctggagttgagctggcgaggcctggggcttcagtgaagctgtcctgcaaggcttctggctacaccttcacaagctatggtataagctgggtgaagcagagaactggacaggcccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagatcggggcctattactacgttagtagctacggactatggtatggactactggggtcaaggaacctcagtcaccgtctcctca

SEQ ID NO: 114 - DNA, 333 bp, Artificial Sequence
  Description of Artificial Sequence Synthetic polynucleotide
gacattgtgctgacacagtctcctgcttccttagctgtatctctggggcagagggccaccatctcctgcagggccagcaaaagtgtcagtacatctagctatagttacatgcactggtaccaacagaaaccaggacagccgcccaaactcctcatcaagtttgcatccaacctagaatctggggtccctgccaggttcagtggcagtgggtctgggacagacttcaccctcaacatccatcctgtggaggaggaggatgctgcaacatattactgtcagcacagtagggagtttccgtacacgttcggaggggggaccaagctggaaataaaa

SEQ ID NO: 115 - DNA, 372 bp, Artificial Sequence
  Description of Artificial Sequence Synthetic polynucleotide
gaggttcagctgcagcagtctgtggcagagcttgtgaggccaggggcctcagtcaagttgtcctgcacagcttctgacttcaatattaaaaacacctatattcactgggtgaaacagaggcctgaacagggcctggagtggattggaaggattgatcctgtgaatggtaatactaaatatgccccgaagttccaggacaaggccactataattgtagacacatattccaacacagcctatctgcagctcagcagcctggcatctgaggacactgccgtctattactgtgctagatggcccataactacgattgtagccacggactggtacttcgatgtctggggcacagggaccacggtcaccgtctcctca

SEQ ID NO: 116 - DNA, 336 bp, Artificial Sequence
  Description of Artificial Sequence Synthetic polynucleotide
gatgttttgatgacccaaactcaactcgccctgcctgtcaggcttggagagcaagcctccatttcttgcagatctagtcagagcattgtaaatagtaatggaaacacctatttagaatggtacctgcagaaaccaggccagtctccaaagctcctgatctacaaagtttccaaccgattttctggggtcccagacaggttcagtggcagtggatcagggacagattttacactcaagatcagcagagtggaggctgaggatctgggagtttattactgctttcaaggttcacatgttccgtggacgttcggtggaggcaccaagctggaaatcaaa

SEQ ID NO: 117 - DNA, 348 bp, Artificial Sequence
  Description of Artificial Sequence Synthetic polynucleotide
caggtccagttgcagcagtctggagctgagctggtaaggcctgggacttcagtgaagatatcctgcaaggcttctggatacgccttcactaactactggctaggttgggtaaagcagaggcctggacatggacttgagtggattggagatatttaccctggaagtggtaatacttactacaatgagaagttcaagggcaaagccacactgactgcagacaaatcctcgagcacagcctatatgcagctcagtagcctgacatctgaggactctgctgtctttttctgtgcagcttacctctatgctatggactactggggtcaaggaacctcagtcaccgtctcctca

SEQ ID NO: 118 - DNA, 318 bp, Artificial Sequence
  Description of Artificial Sequence Synthetic polynucleotide
caaattgttctcacccagtctccagcaatcatgtctgcatctccaggggagaaggtcaccataacctgcagtgccagctcaagtgtaagttacatgcactggttccagcagaagccaggcacttctcccaaactctggatttatagcgcatccaacctggcttctggagtccctgctcgcttcagtggcagtggatctgggacctcttactctctcacaatcagccgaatggaggctgaagatgctgccacttattactgccagcaaaggggtagttacccattcacgttcggctcggggacaaagttggaaataaaa

SEQ ID NO: 119 - DNA, 360 bp, Artificial Sequence
  Description of Artificial Sequence Synthetic polynucleotide
caggtgcaattgaaggagtcaggacctggcctggtggcgccctcgcagagcctgtccattacctgcactgtctctgggctctcattaaacagccatgatataagctggattcgccagccaccaggaaagggtctggagtggcttggagtaatatggagtggtggaggcacaaaatataattcagctttcatgtccagactgagcatcagcaaggacaactccaagagccaagtgttcttaaaaatgaacagtctgcacactgatgacacagccatatattactgtgtaagaaccccctctatgattacttactctgctatggactactggggtcaaggaacctcagtcaccgtctcctca

SEQ ID NO: 120 - DNA, 324 bp, Artificial Sequence
  Description of Artificial Sequence Synthetic polynucleotide
gaaattgtactcacccagtctccagcactcatggctgcatctccaggggagaaggtcaccatcacctgcagtgtcagctcaagtataagttcccgcaacttacactggtaccagcagaagtcacaaacctcccccaaagcctggattcatgccacatccaacctggcttctggagtccctgttcgcttcagtggcagtggatctgggacctcttattctctcacaatcagcagcatggaggctgaagatgctgccacttattactgtcaacagtggagtagttacccactcacgttcggctcggggacaaagttggaaataaaa

SEQ ID NO: 121 - DNA, 354 bp, Artificial Sequence
  Description of Artificial Sequence Synthetic polynucleotide
gaggtccagcttcagcagtcaggacctgagctggtgaaatccggggcctcagtgaagatatcctgcaaggcttctggatacacattcactgaccacaaaatgcactgggtgaagcagagccatggaaagagccttgagtggattggatatatttctccttacaatggtggcactggctacaatcagaagttcaagagcaaggtcacattgactgtagacatatcctccagtacagcctatatggaactccgcagcctgacatctgaagactctgcagtctattactgtgcaagatggggtttctacgatgtaatggactcctggggtcaagggacctcagtcaccgtctcctca

SEQ ID NO: 122 - DNA, 336 bp, Artificial Sequence
  Description of Artificial Sequence Synthetic polynucleotide
gatgttttgttgacccaaactccactctccctgcctgtcagtcttggagatcaagcctccatctcttgcagatctagtcagaccattgttcatattattggagacacctatttagagtggtacctgcagaaaccaggccagtctccaaagctcctgatctacaaagtttccaaccgattttctggggtcccagacaggttcagtggcagtggagcagggacagatttcacactcaagatcagcagagtggaggctgaggatctgggagtttattactgctttcaaggttcacatgttcctcggacgttcggtggaggcaccaagctggaaatcaaa

SEQ ID NO: 123 - DNA, 360 bp, Artificial Sequence
  Description of Artificial Sequence Synthetic polynucleotide
gaagtgaaactggtggagtctgggggaggtttagtgcagcctggagggtccctgaaactctcctgtgcagcctctggattcacttttagtacttataccatgtcttgggttcgccagactccagagaagaggctggagtgggtcgcatacattagttatggtggtggtagcacctcctatccagacactgtaaagggccgattcaccatctccagagacaatgccaagaacaccctttacctacaaatgagcagtctgaagtctgaggacacggccatttattattgtacaagacacgttacggtagttgagaattctatggactactggggtcaaggaacctcagtcaccgtctcctca

SEQ ID NO: 124 - DNA, 321 bp, Artificial Sequence
  Description of Artificial Sequence Synthetic polynucleotide
gacatcttgctgactcagtctccagacatcctgtctgtgagtccaggagaaagagtcagtttctcctgcagggccagccagagcattggcacaagcatacactggtatcagcaaagaacgaatggttctccaaggcttctcataaaatatgtttctgagtctttctctgggaccccttccagatttagtggcagtggatcagggacagcttttactcttaccatcaacagtgtggagtctgaagatattgcagattattactgtcaacaaactcatagctggccgtggacgttcggtggaggcaccaagctggagatcaaa

SEQ ID NO: 125 - DNA, 366 bp, Artificial Sequence
  Description of Artificial Sequence Synthetic polynucleotide
gaggttcagctgcagcagtctggggcagagcttgtgaagccaggggcctcagtcaagttgtcctgcacagcttctggcttcaacattaaaaactactatatgcactgggtgaagcagaggactgaagagggcctggagtggattggaaggattgatcctgaggatggtgaaactaaatatgccccgaaattccaggccaaggccactataacagcagacacatcctccaacacagcctacctgcagctcagcagcctgacatctgaggacactgccgtctatttctgtgctagaaagggctactatggtaactacggggcctggtttgcttactggggccaagggactctggtcactgtctctgca

SEQ ID NO: 126 - DNA, 321 bp, Artificial Sequence
  Description of Artificial Sequence Synthetic polynucleotide
gacatccagatgactcagtctccagcctccctatctgtatctgtgggagaaactgtcaccatcacatgtcgagcaagtgaagatctttacagtaatttagcatggtatcagcagaaacagggaaaatctcctcagctcctggtctatgctgcaacattcttagcagatggtgtgccatcaaggttcagtggcagtggatcaggcacacagtattccctcaagatcaacagcctgcagtctgaagattttgggaattattactgtcaacatttttggggtactcctccgacgttcggtggaggcaccaagctggaaatcaaa

SEQ ID NO: 127 - DNA, 342 bp, Artificial Sequence
  Description of Artificial Sequence Synthetic polynucleotide
caggtccaactgcagcagcctggggctgagcttgtgaagcctggggcttcagtgaagatgtcctgcaaggcttctggctacaccttcaccaggcactggataacctgggtgaagcagaggcctggacaaggccttgagtggattggagatatttttcctggtagtggtagtattaactacaatgagaagttcaagagcgaggccacactgactgtagaaaaatcctccagcacagcctacatgcagctcagcagcctgacatctgaggactctgcggtctattactgtgcaagaggggacgtcgatgtctggggcacagggaccacggtcaccgtctcctca

SEQ ID NO: 128 - DNA, 321 bp, Artificial Sequence
  Description of Artificial Sequence Synthetic polynucleotide
gacatccagatgacccagtctccatcgtccttatctgcctctctgggagaaagagtcagtctcacttgtcgggcaagtcaggaaattcgtggttacttaagctggcttcagcagaaaccaaatggaactattaaacgcctgatctacgccgcatccactttagattctggtgtcccaaaaaggttcagtgccactaggtctgggtcagatttttctctcaccatcagcagccttgagtctgaagattttgcagactattactgtctccaatatgctagttatccattcacgttcggctcggggacaaagttggaaataaaa

SEQ ID NO: 129 - DNA, 369 bp, Artificial Sequence
  Description of Artificial Sequence Synthetic polynucleotide
gaagtgcagttggtggagtctgggggaggcttagtgacgcctggagggtccctgaaactctcctgtgtcgcctctggattcgctttcagtacctatggcatgtcttgggttcgccagactccggagaggaggctggagtgggtcgcatatattagtggtggtggtggaagtatctactatccagacagtgtgaagggccgattcaccatctccagagacaatgccaagaacatcctgtaccttcaaatgagcagtctgaagtctgaggacacagccatgtatcactgtaccagacaccccgatttattactacggtatgattatgcattggaccactggggtcaaggaacctcagtcaccgtctcctca

SEQ ID NO: 130 - DNA, 321 bp, Artificial Sequence
  Description of Artificial Sequence Synthetic polynucleotide
gacatccagatgactcagtctccagcctccctatctgcatctgtgggagaaactgtcaccatcacatgtcgagcaagtgaaaatattcacatttatttagcatggtatcagcagaaacagggaaaatctcctcagctcctggtctataatggaaaaaccttagcagatggtgtgccatcaaggttcagtggcagtggatcaggaacacaatattctctcaagatcaataccctgcagcctgaagattttgggaattattactgtcaacatttttggaatagtacgtggacgttcggtggaggcaccaagctggaaatcaaa

SEQ ID NO: 131 - DNA, 456 bp, Homo sapiens
gccgtccagggccccgaggagaccgtgacccaggactgtctccagctcatcgctgactccgaaacccccaccatccagaagggcagctacaccttcgtgccttggctgctgagcttcaagagaggcagcgccctggaggagaaggagaacaagattctcgtgaaggagaccggctacttcttcatctacggccaggtgctctacaccgacaagacctatgccatgggacacctcatccagaggaagaaggtccacgtcttcggcgacgagctcagcctggtcaccctgttcaggtgcatccagaatatgcccgagaccctgcccaataacagctgctacagcgctggcattgccaaactggaggagggcgacgaactgcagctcgccatccccagggagaacgcccagatcagcctcgacggcgatgtgaccttcttcggcgccctcaagctcctg

SEQ ID NO: 132 - DNA, 858 bp, Homo sapiens
atggacgactctacagagcgcgagcagtctaggctcacatcatgtctcaagaagcgggaagagatgaagctcaaagaatgcgtcagcatcctgcctagaaaggaatcaccttccgtccggtcttccaaggatggaaagctgctcgccgctacactgctcctcgcactcctgtcttgttgcctcacagtcgtgtcattttaccaggtggccgcactacagggcgatctggcatcactgcgcgcagagctacaaggccaccacgctgagaagctcccagccggggctggggcaccaaaggccggactcgaagaggcacccgcagtgaccgccggactgaaaatttttgagccacccgcccccggcgagggcaattctagccagaactcacacaacaagagagcagtgcagggccccgaggaaaccgtgacacaggattgtctccagctgattgccgatagcgagaccccaactattcagaagggatcatacacatttgtgccttggctcctgagcttcaagcgcgggtccgccttagaggaaaaggaaaataagattctggtcaaagaaaccgggtacttctttatctacggacaggtcctgtacaccgataagacatacgctatgggacacctgatccagcggaagaaggtgcacgtcttcggcgatgagctatctctggtgactctgtttcggtgtattcagaatatgcctgagaccctccctaacaattcatgttactccgctggaattgctaagctcgaagagggggatgaactacagctggctatcccacgcgagaacgcacagattagcctcgacggcgacgtgactttcttcggcgccctcaagctgctgtga

SEQ ID NO: 133 - DNA, 858 bp, Macaca fascicularis
atggacgactctacagagcgcgagcagtcacggctgacatcatgtctcaagaagagagaagagatgaagctgaaagaatgcgtctcaattctgccacagaaggaatccccatccgtgcggtctagcaaggatagaaagctcctcgcagccgccctgctgctcgccctgctgtcatgttgcctgatggtcgtgtccttctaccaggtggccgctctccagggcgatctggcatcactgcgcgccgagttgcagtcccaccacgcagagaagctcccagcccgagcacgggccccaaaggcaggcctcggcgaggcacccgcagtgaccgccggactcaaaatctttgagccacccgcacctggcgagggaaattctagccagtcttcacacaataagcgcgctatccagggggccgacgagaccgtgattcaggattgtctccagctgattgccgatagcgagaccccaactattcagaagggatcatacacatttgtgccttggctcctgagcttcaagcgcgggtccgccttagaggaaaaggaaaataagattctggtcaaagaaaccgggtacttctttatctacggacaggtcctgtacaccgataagacatacgctatgggacacctgatccagcggaagaaggtgcacgtcttcggcgatgagctatctctggtgactctgtttcggtgtattcagaatatgcctgagaccctccctaacaattcatgttactccgctggaattgctaagctcgaagagggggatgaactacagctggctatcccacgcgagaacgcacagattagcctcgacggcgacgtgactttcttcggcgccctcaagctgctgtga

SEQ ID NO: 134 - DNA, 18 bp, Artificial Sequence
  Description of Artificial Sequence Synthetic oligonucleotide
  pTT-EF1a-F
gccctttttgagtttgga

SEQ ID NO: 135 - DNA, 18 bp, Artificial Sequence
  Description of Artificial Sequence Synthetic oligonucleotide
  pSV40
cactgcattctagttgtg

SEQ ID NO: 136 - protein, 109 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARWGQGTTVTVSS

SEQ ID NO: 137 - protein, 104 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCFGGGTKVEIK

SEQ ID NO: 138 - protein, 109 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARWGQGTTVTVSS

SEQ ID NO: 139 - protein, 98 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCFGGGTKVEIK

SEQ ID NO: 140 - protein, 125 aa, Artificial Sequence
  Description of Artificial Sequence Synthetic polypeptide
EVQLVQSGAEVKKPGASVKVSCKASGYTFTSYGISWVRQAPGQGLEWMGEIYPRSGNTYYNEKFKGRVTMTTDTSTSTAYMELRSLRSDDTAVYYCARSGPITTLVATDYGMDYWGQGTTVTVSS

SEQ ID NO: 141 - protein, 125 aa, Artificial Sequence
  Description of Artificial Sequence Synthetic polypeptide
EVQLVQSGAEVKKPGASVKVSCKASGYTFTSYGISWVRQAPGQGLEWMGEIYPRSGNTYYNEKFKGRVTMTADTSTSTAYMELRSLRSDDTAVYYCARSGPITTLVATDYGMDYWGQGTTVTVSS

SEQ ID NO: 142 - protein, 125 aa, Artificial Sequence
  Description of Artificial Sequence Synthetic polypeptide
EVQLVQSGAEVKKPGASVKVSCKASGYTFTSYGISWVRQAPGQGLEWMGEIYPRSGNTYYNEKFKGRVTLTADTSTSTAYMELRSLRSDDTAVYYCARSGPITTLVATDYGMDYWGQGTTVTVSS

SEQ ID NO: 143 - protein, 125 aa, Artificial Sequence
  Description of Artificial Sequence Synthetic polypeptide
EVQLVQSGAEVKKPGASVKVSCKASGYTFTSYGISWVRQAPGQGLEWIGEIYPRSGNTYYNEKFKGRVTLTADTSTSTAYMELRSLRSDDTAVYYCARSGPITTLVATDYGMDYWGQGTTVTVSS

SEQ ID NO: 144 - protein, 125 aa, Artificial Sequence
  Description of Artificial Sequence Synthetic polypeptide
EVQLVQSGAEVKKPGASVKVSCKASGYTFTSYGISWVKQAPGQGLEWIGEIYPRSGNTYYNEKFKGRVTLTADTSTSTAYMELRSLRSDDTAVYYCARSGPITTLVATDYGMDYWGQGTTVTVSS

SEQ ID NO: 145 - protein, 125 aa, Artificial Sequence
  Description of Artificial Sequence Synthetic polypeptide
EVQLVQSGAEVKKPGASVKVSCKASGYTFTSYGISWVKQAPGQGLEWIGEIYPRSGNTYYNEKFKGKATLTADTSTSTAYMELRSLRSDDTAVYYCARSGPITTLVATDYGMDYWGQGTTVTVSS

SEQ ID NO: 146 - protein, 125 aa, Artificial Sequence
  Description of Artificial Sequence Synthetic polypeptide
EVQLVQSGAEVKKPGASVKVSCKASGYTFTSYGISWVKQAPGQALEWIGEIYPRSGNTYYNEKFKGKATLTADKSTSTAYMELRSLRSDDTAVYFCARSGPITTLVATDYGMDYWGQGTTVTVSS

SEQ ID NO: 147 - protein, 111 aa, Artificial Sequence
  Description of Artificial Sequence Synthetic polypeptide
DIVMTQSPDSLAVSLGERATINCRASKSVSTSSYSYMHWYQQKPGQPPKLLIKFASNLESGVPDRFSGSGSGTDFTLTISSLQAEDVAVYYCQHSREFPYTFGGGTKVEIK

SEQ ID NO: 148 - protein, 111 aa, Artificial Sequence
  Description of Artificial Sequence Synthetic polypeptide
DIVLTQSPDSLAVSLGERATINCRASKSVSTSSYSYMHWYQQKPGQPPKLLIKFASNLESGVPDRFSGSGSGTDFTLTISSLQAEDVAVYYCQHSREFPYTFGGGTKVEIK

SEQ ID NO: 149 - protein, 120 aa, Artificial Sequence
  Description of Artificial Sequence Synthetic polypeptide
EVQLVESGGGLVQPGGSLRLSCAASGFTFSTYTMSWVRQAPGKGLEWVAYISYGGGSTSYPDTVKGRFTISRDNAKNSLYLQMNSLRAEDTAVYYCARHVTVVENSMDYWGQGTTVTVSS

SEQ ID NO: 150 - protein, 120 aa, Artificial Sequence
  Description of Artificial Sequence Synthetic polypeptide
EVQLVESGGGLVQPGGSLRLSCAASGFTFSTYTMSWVRQAPGKGLEWVAYISYGGGSTSYPDTVKGRFTISRDNAKNSLYLQMNSLRAEDTAVYYCTRHVTVVENSMDYWGQGTTVTVSS

SEQ ID NO: 151 - protein, 120 aa, Artificial Sequence
  Description of Artificial Sequence Synthetic polypeptide
EVQLVESGGGLVQPGGSLRLSCAASGFTFSTYTMSWVRQAPGKRLEWVAYISYGGGSTSYPDTVKGRFTISRDNAKNSLYLQMNSLRAEDTAVYYCTRHVTVVENSMDYWGQGTTVTVSS

SEQ ID NO: 152 - protein, 120 aa, Artificial Sequence
  Description of Artificial Sequence Synthetic polypeptide
EVQLVESGGGLVQPGGSLRLSCAASGFTFSTYTMSWVRQAPEKRLEWVAYISYGGGSTSYPDTVKGRFTISRDNAKNSLYLQMNSLRAEDTAVYYCTRHVTVVENSMDYWGQGTTVTVSS

SEQ ID NO: 153 - protein, 107 aa, Artificial Sequence
  Description of Artificial Sequence Synthetic polypeptide
EIVLTQSPDFQSVTPKEKVTITCRASQSIGTSIHWYQQKPDQSPKLLIKYVSESFSGVPSRFSGSGSGTDFTLTINSLEAEDAATYYCQQTHSWPWTFGGGTKVEIK

SEQ ID NO: 154 - protein, 107 aa, Artificial Sequence
  Description of Artificial Sequence Synthetic polypeptide
EIVLTQSPDFQSVTPKEKVTITCRASQSIGTSIHWYQQKPDQSPKLLIKYVSESFSGTPSRFSGSGSGTDFTLTINSLEAEDAATYYCQQTHSWPWTFGGGTKVEIK

SEQ ID NO: 155 - protein, 107 aa, Artificial Sequence
  Description of Artificial Sequence Synthetic polypeptide
DILLTQSPDFQSVTPKEKVTITCRASQSIGTSIHWYQQKPDQSPKLLIKYVSESFSGTPSRFSGSGSGTDFTLTINSLEAEDAATYYCQQTHSWPWTFGGGTKVEIK

SEQ ID NO: 156 - protein, 107 aa, Artificial Sequence
  Description of Artificial Sequence Synthetic polypeptide
EIVLTQSPDFQSVTPKEKVTITCRASQSIGTSIHWYQQKTDQSPKLLIKYVSESFSGTPSRFSGSGSGTDFTLTINSLEAEDAATYYCQQTHSWPWTFGGGTKVEIK

SEQ ID NO: 157 - protein, 107 aa, Artificial Sequence
  Description of Artificial Sequence Synthetic polypeptide
DILLTQSPDFQSVTPKEKVTITCRASQSIGTSIHWYQQKTDQSPKLLIKYVSESFSGTPSRFSGSGSGTDFTLTINSLEAEDAATYYCQQTHSWPWTFGGGTKVEIK

SEQ ID NO: 158 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgcggcaggccccaggacagggactcgagtggatgggagaaatctacccccggagcggcaacacctactacaacgagaagttcaagggccgcgtgaccatgacaaccgatacaagcaccagcaccgcctacatggagctgagaagcctgaggagcgacgacacagccgtgtactattgcgccaggagcggacctatcaccacactggtggccaccgactacggcatggactattggggccagggaaccacagtgaccgtgtctagc

SEQ ID NO: 159 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgcggcaggccccaggacagggactcgagtggatgggagaaatctacccccggagcggcaacacctactacaacgagaagttcaagggccgcgtgaccatgacagccgatacaagcaccagcaccgcctacatggagctgagaagcctgaggagcgacgacacagccgtgtactattgcgccaggagcggacctatcaccacactggtggccaccgactacggcatggactattggggccagggaaccacagtgaccgtgtctagc

SEQ ID NO: 160 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgcggcaggccccaggacagggactcgagtggatgggagaaatctacccccggagcggcaacacctactacaacgagaagttcaagggccgcgtgaccctgacagccgatacaagcaccagcaccgcctacatggagctgagaagcctgaggagcgacgacacagccgtgtactattgcgccaggagcggacctatcaccacactggtggccaccgactacggcatggactattggggccagggaaccacagtgaccgtgtctagc

SEQ ID NO: 161 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgcggcaggccccaggacagggactcgagtggatcggagaaatctacccccggagcggcaacacctactacaacgagaagttcaagggccgcgtgaccctgacagccgatacaagcaccagcaccgcctacatggagctgagaagcctgaggagcgacgacacagccgtgtactattgcgccaggagcggacctatcaccacactggtggccaccgactacggcatggactattggggccagggaaccacagtgaccgtgtctagc

SEQ ID NO: 162 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgaagcaggccccaggacagggactcgagtggatcggagaaatctacccccggagcggcaacacctactacaacgagaagttcaagggccgcgtgaccctgacagccgatacaagcaccagcaccgcctacatggagctgagaagcctgaggagcgacgacacagccgtgtactattgcgccaggagcggacctatcaccacactggtggccaccgactacggcatggactattggggccagggaaccacagtgaccgtgtctagc

SEQ ID NO: 163 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgaagcaggccccaggacagggactcgagtggatcggagaaatctacccccggagcggcaacacctactacaacgagaagttcaagggcaaggccaccctgacagccgatacaagcaccagcaccgcctacatggagctgagaagcctgaggagcgacgacacagccgtgtactattgcgccaggagcggacctatcaccacactggtggccaccgactacggcatggactattggggccagggaaccacagtgaccgtgtctagc

SEQ ID NO: 164 - DNA, 375 bp, Artificial Sequence
  Description of Artificial Sequence Synthetic polynucleotide
gaagtgcagctggtgcagagcggagcagaagtgaagaagccaggcgcaagcgtgaaggtgtcttgcaaggccagcggctacaccttcaccagctacggcatctcttgggtgaagcaggccccaggacaggcactcgagtggatcggagaaatctacccccggagcggcaacacctactacaacgagaagttcaagggcaaggccaccctgacagccgataagagcaccagcaccgcctacatggagctgagaagcctgaggagcgacgacacagccgtgtacttttgcgccaggagcggacctatcaccacactggtggccaccgactacggcatggactattggggccagggaaccacagtgaccgtgtctagc

SEQ ID NO: 165 - DNA, 333 bp, Artificial Sequence
  Description of Artificial Sequence Synthetic polynucleotide
gacatcgtgatgacccagagcccagacagcctggcagtgtctctgggagagagagccacaatcaattgccgggccagcaagagcgtgtccaccagcagctacagctacatgcattggtatcagcagaaaccaggccagcctcctaagctgctgatcaagttcgccagcaacctggagagcggcgtgccagatagattcagcggaagcggaagcggcaccgatttcaccctgaccatcagctctctgcaggccgaagacgtggccgtgtattattgccagcacagccgggagttcccctacacatttggcggcggcaccaaggtggagatcaag

SEQ ID NO: 166 - DNA, 333 bp, Artificial Sequence
  Description of Artificial Sequence Synthetic polynucleotide
gacatcgtgctgacccagagcccagacagcctggcagtgtctctgggagagagagccacaatcaattgccgggccagcaagagcgtgtccaccagcagctacagctacatgcattggtatcagcagaaaccaggccagcctcctaagctgctgatcaagttcgccagcaacctggagagcggcgtgccagatagattcagcggaagcggaagcggcaccgatttcaccctgaccatcagctctctgcaggccgaagacgtggccgtgtattattgccagcacagccgggagttcccctacacatttggcggcggcaccaaggtggagatcaag

SEQ ID NO: 167 - DNA, 360 bp, Artificial Sequence
  Description of Artificial Sequence Synthetic polynucleotide
gaggtgcagctggtggaatcaggaggaggactggtgcagccaggaggatctctgagactgtcttgcgccgccagcggctttacattcagcacctacaccatgtcttgggtccggcaggctccaggcaaaggactcgagtgggtggcctacatcagctacggcggcggctctacaagctacccagacaccgtgaagggcaggttcaccatcagccgggacaacgccaagaacagcctgtacctgcagatgaacagcctgagggccgaggataccgccgtgtactattgcgctaggcacgtgaccgtggtggagaacagcatggactattggggccagggaaccaccgtgacagtgtcttct

SEQ ID NO: 168 - DNA, 360 bp, Artificial Sequence
  Description of Artificial Sequence Synthetic polynucleotide
gaggtgcagctggtggaatcaggaggaggactggtgcagccaggaggatctctgagactgtcttgcgccgccagcggctttacattcagcacctacaccatgtcttgggtccggcaggctccaggcaaaggactcgagtgggtggcctacatcagctacggcggcggctctacaagctacccagacaccgtgaagggcaggttcaccatcagccgggacaacgccaagaacagcctgtacctgcagatgaacagcctgagggccgaggataccgccgtgtactattgcaccaggcacgtgaccgtggtggagaacagcatggactattggggccagggaaccaccgtgacagtgtcttct

SEQ ID NO: 169 - DNA, 360 bp, Artificial Sequence
  Description of Artificial Sequence Synthetic polynucleotide
gaggtgcagctggtggaatcaggaggaggactggtgcagccaggaggatctctgagactgtcttgcgccgccagcggctttacattcagcacctacaccatgtcttgggtccggcaggctccaggcaaaagactcgagtgggtggcctacatcagctacggcggcggctctacaagctacccagacaccgtgaagggcaggttcaccatcagccgggacaacgccaagaacagcctgtacctgcagatgaacagcctgagggccgaggataccgccgtgtactattgcaccaggcacgtgaccgtggtggagaacagcatggactattggggccagggaaccaccgtgacagtgtcttct

SEQ ID NO: 170 - DNA, 360 bp, Artificial Sequence
  Description of Artificial Sequence Synthetic polynucleotide
gaggtgcagctggtggaatcaggaggaggactggtgcagccaggaggatctctgagactgtcttgcgccgccagcggctttacattcagcacctacaccatgtcttgggtccggcaggctccagagaagaggctcgagtgggtggcctacatcagctacggcggcggctctacaagctacccagacaccgtgaagggcaggttcaccatcagccgggacaacgccaagaacagcctgtacctgcagatgaacagcctgagggccgaggataccgccgtgtactattgcacaaggcacgtgaccgtggtggagaacagcatggactattggggccagggaaccaccgtgacagtgtcttct

SEQ ID NO: 171 - DNA, 321 bp, Artificial Sequence
  Description of Artificial Sequence Synthetic polynucleotide
gagatcgtgctgacccagagcccagacttccagtcagtgacccccaaggagaaggtcaccatcacttgcagagccagccagagcatcggcaccagcatccattggtatcagcagaagcccgaccagagccctaagctgctgatcaagtacgtgtccgagagctttagcggcgtgcctagcagattcagcggcagcggcagcggcacagacttcaccctgaccatcaacagcctggaggcagaggacgcagccacctactattgccagcagacccactcttggccttggaccttcggaggaggcaccaaggtggagatcaag

SEQ ID NO: 172 - DNA, 321 bp, Artificial Sequence
  Description of Artificial Sequence Synthetic polynucleotide
gagatcgtgctgacccagagcccagacttccagtcagtgacccccaaggagaaggtcaccatcacttgcagagccagccagagcatcggcaccagcatccattggtatcagcagaagcccgaccagagccctaagctgctgatcaagtacgtgtccgagagctttagcggcacacctagcagattcagcggcagcggcagcggcacagacttcaccctgaccatcaacagcctggaggcagaggacgcagccacctactattgccagcagacccactcttggccttggaccttcggaggaggcaccaaggtggagatcaag

SEQ ID NO: 173 - DNA, 321 bp, Artificial Sequence
  Description of Artificial Sequence Synthetic polynucleotide
gacatcttgctgacccagagcccagacttccagtcagtgacccccaaggagaaggtcaccatcacttgcagagccagccagagcatcggcaccagcatccattggtatcagcagaagcccgaccagagccctaagctgctgatcaagtacgtgtccgagagctttagcggcacccctagcagattcagcggcagcggcagcggcacagacttcaccctgaccatcaacagcctggaggcagaggacgcagccacctactattgccagcagacccactcttggccttggaccttcggaggaggcaccaaggtggagatcaag

SEQ ID NO: 174 - DNA, 321 bp, Artificial Sequence
  Description of Artificial Sequence Synthetic polynucleotide
gagatcgtgctgacccagagcccagacttccagtcagtgacccccaaggagaaggtcaccatcacttgcagagccagccagagcatcggcaccagcatccattggtatcagcagaagaccgaccagagccctaagctgctgatcaagtacgtgtccgagagctttagcggcacccctagcagattcagcggcagcggcagcggcacagacttcaccctgaccatcaacagcctggaggcagaggacgcagccacctactattgccagcagacccactcttggccttggaccttcggaggaggcaccaaggtggagatcaag

SEQ ID NO: 175 - DNA, 321 bp, Artificial Sequence
  Description of Artificial Sequence Synthetic polynucleotide
gacatcttgctgacccagagcccagacttccagtcagtgacccccaaggagaaggtcaccatcacttgcagagccagccagagcatcggcaccagcatccattggtatcagcagaagaccgaccagagccctaagctgctgatcaagtacgtgtccgagagctttagcggcacccctagcagattcagcggcagcggcagcggcacagacttcaccctgaccatcaacagcctggaggcagaggacgcagccacctactattgccagcagacccactcttggccttggaccttcggaggaggcaccaaggtggagatcaag

SEQ ID NO: 176 - DNA, 42 bp, Artificial Sequence
  Description of Artificial Sequence Synthetic primer
  2-1G11-VH-forward primer
gattcttaagggtgtccagtgcgaggtgcagctggtggagtc

SEQ ID NO: 177 - DNA, 41 bp, Artificial Sequence
  Description of Artificial Sequence Synthetic primer
  2-1G11-VH-reverse primer
gatgggcccttggtcgaagctgaggagacggtgaccgtggt

SEQ ID NO: 178 - DNA, 43 bp, Artificial Sequence
  Description of Artificial Sequence Synthetic primer
  2-1G11-VL-forward primer
tggttccccggctcgcgatgcgaaattgtgatgactcagtctc

SEQ ID NO: 179 - DNA, 45 bp, Artificial Sequence
  Description of Artificial Sequence Synthetic primer
  2-1G11-VL-reverse primer
cagatggtgcagccaccgtacgtttgatctccaccttggtccctc

SEQ ID NO: 180 - DNA, 44 bp, Artificial Sequence
  Description of Artificial Sequence Synthetic primer
  L9G7-VH-forward primer
gattcttaagggtgtccagtgccaaatccagctggtacaatctg

SEQ ID NO: 181 - DNA, 42 bp, Artificial Sequence
  Description of Artificial Sequence Synthetic primer
  L9G7-VH-reverse primer
gatgggcccttggtcgaagctgaggagacggtgaccgtggtc

SEQ ID NO: 182 - DNA, 45 bp, Artificial Sequence
  Description of Artificial Sequence Synthetic primer
  L9G7-VL-forward primer
tggttccccggctcgcgatgcgaaattgtgttgacgcagtctcca

SEQ ID NO: 183 - DNA, 45 bp, Artificial Sequence; identical to SEQ ID NO: 179
  Description of Artificial Sequence Synthetic primer
  L9G7-VL-reverse primer

SEQ ID NO: 184 - DNA, 44 bp, Artificial Sequence
  Description of Artificial Sequence Synthetic primer
  L1D12-VH-forward primer
gattcttaagggtgtccagtgccaggtccagctggtgcagtctg

SEQ ID NO: 185 - DNA, 42 bp, Artificial Sequence; identical to SEQ ID NO: 181
  Description of Artificial Sequence Synthetic primer
  L1D12-VH-reverse primer

SEQ ID NO: 186 - DNA, 41 bp, Artificial Sequence
  Description of Artificial Sequence Synthetic primer
  L1D12-VL-forward primer
actgcacagggtctctctcctcttctgagctgactcaggac

SEQ ID NO: 187 - DNA, 45 bp, Artificial Sequence
  Description of Artificial Sequence Synthetic primer
  L1D12-VL-reverse primer
gagggggcagccttgggttgacctaggacggtcaacttggtccct